(19) United States
(12) Patent Application Publication
(10) Pub. No.: US 2022/0399128 A1
(43) Pub. Date: Dec. 15, 2022

(54) TECHNIQUES FOR DETERMINING RENAL PATHOPHYSIOLOGIES

(71) Applicant: Fresenius Medical Care Holdings, Inc., Waltham, MA (US)

(72) Inventors: Alhaji Cherif, Kew Gardens, NY (US); Peter Kotanko, New York, NY (US); Paulo Galuzio, New York, NY (US)

(21) Appl. No.: 17/829,697

(22) Filed: Jun. 1, 2022

Related U.S. Application Data
(60) Provisional application No. 63/195,818, filed on Jun. 2, 2021.

(51) Int. Cl. G16H 50/50; G16H 50/20; G16H 10/40; G16H 10/60; G06N 5/04

(52) U.S. Cl. CPC G16H 50/50; G16H 50/20; G16H 10/40; G16H 10/60; G06N 5/046

(57) ABSTRACT

The described technology may include processes to model renal pathophysiology in patients and/or patient populations. In one embodiment, a method may include a CKD/ESRD condition analysis, such as a vascular calcification analysis. The method may include, via a processor of a computing device: determining a vascular calcification model configured to model vascular calcification of a virtual patient to determine a causal relationship between at least one patient characteristic and a vascular calcification indicator, and generating a causal relationship structure configured to visualize a causal relationship between the at least one patient characteristic and the vascular calcification indicator. Other embodiments are described.

### CROSS-REFERENCE TO RELATED APPLICATIONS

This application claims the benefit to U.S. Provisional Application No. 63/195,818, filed Jun. 2, 2021, the entire contents of which are incorporated herein by reference in their entirety.

### BACKGROUND

Patients with chronic kidney and/or end-stage renal diseases (CKD/ESRD) are subject to increased health and morbidity risks than the general population. For example, cardiovascular disease (CVD), accelerated by cardiometabolic risks, is a leading cause of mortality in patients with CKD/ESRD. CKD/ESRD patients are at a higher risk of mortality about two to ten times that of the general population. This risk is not entirely accounted for by traditional risk factors such as hypertension, diabetes, and hyperlipidemia alone; non-traditional risk factors related to CKD metabolic bone disorder (CKD-MBD) and vascular calcification have provided additional missing links. Despite all of these risk factors, cardiovascular disease in CKD/ESRD patients remains underdiagnosed and undertreated. A primary reason is that standard clinical interventions used in the general population for managing cardiovascular disease are ineffective in reducing mortality risk in CKD/ESRD patients, due to interactions between interlinked pathophysiological mechanisms with nonlinear cascading effects. A similar situation exists for other disorders and health conditions, such as anemia, bone mineral metabolism, acid-base disorders, and/or the like.

It is with respect to these and other considerations that the present improvements may be useful.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In one embodiment, an apparatus may include at least one processor and a memory coupled to the at least one processor. The memory may include instructions that, when executed by the at least one processor, cause the at least one processor to perform a chronic kidney and/or end-stage renal diseases (CKD/ESRD) condition analysis process to determine a CKD/ESRD condition model configured to model a CKD/ESRD condition, the vascular calcification analysis process to: receive input data associated with at least one patient, perform a dynamical system learner process to build a collection of dynamical system models, determine a model rank for at least a portion of the collection of dynamical system models, and determine an optimal dynamical system model for modeling the CKD/ESRD condition for the at least one patient.

In some embodiments of the apparatus, the instructions, when executed by the at least one processor, may cause the at least one processor to pre-process the input data to impute missing values. In various embodiments of the apparatus, the instructions, when executed by the at least one processor, may cause the at least one processor to perform a causal analysis of the input data to generate causal information. In some embodiments of the apparatus, the causal information may include a causal diagram. In various embodiments of the apparatus, the model rank may be configured to indicate model performance for dynamical relationships between variables in the input data.

In exemplary embodiments of the apparatus, the collection of dynamical system models may model one or more of the following variables: pre-treatment pulse pressure (P), Neutrophils-Lymphocytes ratio (ρ_{NL}), Serum calcium concentration (C_{Ca}), Intact Parathyroid Hormone (C_{PTH}), Serum albumin concentration (g/dL) (C_{Ab}), Serum phosphorus concentration (C_{P}), or Alkaline Phosphatase (C_{AP}).

In various embodiments of the apparatus, the instructions, when executed by the at least one processor, may cause the at least one processor to: receive patient information for a patient; analyze the patient information using one of the collections of dynamical models to predict a CKD/ESRD condition process for the patient based on modeled variables.

In various embodiments of the apparatus, the input data may include a time series of system observables and a library of functions configured as an operator on the input data. In some embodiments of the apparatus, the dynamical system models may include differential equations that describe a time evolution of at least one variable of the input data.

In one embodiment, a computer-implemented method may be configured to perform a chronic kidney and/or end-stage renal diseases (CKD/ESRD) condition analysis process to determine a CKD/ESRD condition model configured to model a CKD/ESRD condition. The method may include receiving input data associated with at least one patient, performing a dynamical system learner process to build a collection of dynamical system models, determining a model rank for at least a portion of the collection of dynamical system models, and determining an optimal dynamical system model for modeling the CKD/ESRD condition for the at least one patient.

In some embodiments of the method, the method may include pre-processing the input data to impute missing values. In various embodiments of the method, the method may include performing a causal analysis of the input data to generate causal information. In some embodiments of the method, the causal information may include a causal diagram. In various embodiments of the method, the model rank may be configured to indicate model performance for dynamical relationships between variables in the input data.

In exemplary embodiments of the method, the collection of dynamical system models may model one or more of the following variables: pre-treatment pulse pressure (P), Neutrophils-Lymphocytes ratio (ρ_{NL}), Serum calcium concentration (C_{Ca}), Intact Parathyroid Hormone (C_{PTH}), Serum albumin concentration (g/dL) (C_{Ab}), Serum phosphorus concentration (C_{P}), or Alkaline Phosphatase (C_{AP}).

In one embodiment, a computer-implemented method of vascular calcification analysis may include, via a processor of a computing device: determining a vascular calcification model configured to model vascular calcification of a virtual patient to determine a causal relationship between at least one patient characteristic and a vascular calcification indicator; and generate a causal relationship structure configured to visualize a causal relationship between the at least one patient characteristic and the vascular calcification indicator.

In some embodiments of the method, the vascular calcification indicator may include one of pulse pressure (PP) or pulse wave velocity. In some embodiments of the method, the at least one patient characteristic may include at least one of parathyroid hormones (PTH), calcium (Ca), phosphate (PO4), calcium-phosphate product (CaPO4), neutrophil-lymphocyte ratio (NLR), and albumin (Alb). In some embodiments of the method, the causal relationship structure may include at least one of a causality fingerprint or a causality pathway map. In some embodiments of the method, the method may include administering a treatment regimen based on the causal relationship structure.

### BRIEF DESCRIPTION OF THE DRAWINGS

By way of example, specific embodiments of the disclosed machine will now be described, with reference to the accompanying drawings, in which:

FIG. 1 illustrates a first exemplary operating environment in accordance with the present disclosure;

FIG. 2 illustrates the processes and conditions of vascular calcification;

FIG. 3 depicts an illustrative and non-limiting vascular calcification analysis process in accordance with the present disclosure;

FIGS. 4A and 4B depict illustrative and non-limiting examples of modeling approaches in accordance with the present disclosure;

FIGS. 5A and 5B depict illustrative and non-limiting examples of causation output of a vascular calcification model in accordance with the present disclosure;

FIG. 6 depicts illustrative clinical data used to determine the output shown in FIGS. 5A and 5B in accordance with the present disclosure;

FIG. 7 depicts a schematic representation of data in accordance with the present disclosure;

FIG. 8 illustrates an illustrative vascular calcification analysis processes according to a dynamical system learner process in accordance with the present disclosure;

FIG. 9 illustrates exemplary causal diagrams according to the present disclosure;

FIG. 10 illustrates exemplary pre-processing according to the present disclosure;

FIG. 11 depicts illustrative Gaussian processes in accordance with the present disclosure;

FIG. 12A illustrates an example of a causality analysis in accordance with the present disclosure;

FIG. 12B illustrates an example of a causality matrix in accordance with the present disclosure;

FIG. 13 illustrates an exemplary dynamical system learner in accordance with the present disclosure;

FIG. 14 illustrates an example of a dynamical system model process in accordance with the present disclosure

FIG. 15 illustrates an exemplary model ranking process according to the present disclosure;

FIG. 16 illustrates an exemplary vascular calcification analysis processes according to a second embodiment in accordance with the present disclosure;

FIG. 17 depicts a first model discovery example using processes in accordance with the present disclosure;

FIGS. 18A and 18B depict a second model discovery example using processes in accordance with the present disclosure;

FIGS. 19A and 19B depict a third model discovery example using processes in accordance with the present disclosure;

FIG. 20 depicts graphical representations of different model performances in accordance with the present disclosure;

FIGS. 21-36 depict experimental results for a dynamical system learner process in accordance with the present disclosure;

FIG. 37 provides a listing of symbol definitions used in various processes according to the present disclosure; and

FIG. 38 illustrates an embodiment of computing architecture in accordance with the present disclosure.

### DETAILED DESCRIPTION

The present embodiments will now be described more fully hereinafter with reference to the accompanying drawings, in which several exemplary embodiments are shown. The subject matter of the present disclosure, however, may be embodied in many different forms and should not be construed as limited to the embodiments set forth herein. Rather, these embodiments are provided so that this disclosure will be thorough and complete, and will fully convey the scope of the subject matter to those skilled in the art. In the drawings, like numbers refer to like elements throughout.

The described technology is generally directed to processes, systems, and methods for the analysis of conditions of chronic kidney and/or end-stage renal diseases (CKD/ESRD) patients. A non-limiting example of CKD/ESRD conditions may include vascular calcification. In accordance with various aspects of the described embodiments, a method of CKD/ESRD conditions analysis may include a model generation process operative to provide a physiology-based model configured to model the CKD/ESRD conditions of a patient. The models may be generated, determined, or otherwise processed using a dynamical system modeler process or a dynamical system learner process.

In various embodiments, the model generation process may operate using an application that infers a model representation of a dynamical system from noisy time series data with missing values. Such a dynamical system may be represented by a set of ordinary or partial differential equations with or without time delays. From this model representation, important properties about the dynamical evolution of the system may be extracted or otherwise determined, such as causal relationships between the variables. Also, the processes, algorithms, equations, graphs, and/or the like obtained via the model generation process may be used to make predictions about a (real or virtual) biological system based on the present values of the state variables.

In one embodiment, the model generation process may operate to, given a set of noisy time-dependent data on multiple relevant variables, sampled from different realizations and at different times, construct a dynamical system model that best represents the observed data and that allows the determination of causal relations between the variables.

Conventional techniques have proven inadequate for the diagnosis and treatment of CKD/ESRD conditions. Non-limiting examples of CKD/ESRD conditions may include vascular calcification, anemia, bone mineral metabolism disorders, acid-base disorders, and/or the like. For instance, with respect to vascular calcification, CKD/ESRD patients experience up to 30-fold higher cardiovascular disease mortality than the general population. The risks include traditional factors such as age, diabetes mellitus, dyslipidemia, and inflammation, as well as CKD-specific risk factors such as bone mineral metabolism (BMM) disturbances and associated therapies. Increased cardiovascular risk is multifactorial and is due partly to pathophysiological processes specific to CKD, making prevention of cardiovascular disease (CVD) by standard interventions directed at single traditional risks difficult. Currently, there is no broad applicable clinical strategy available to ameliorate the development or progression of vascular calcification and many of the therapeutic clinical studies have shown poor results.

In some embodiments, for example, a vascular calcification model may be provided that is operative to disentangle the prevalent underlying physiological mechanisms that accelerate the risk of cardiovascular events associated with CKD-metabolic bone disorder (CKD-MBD) using mathematical models. A vascular calcification model may operate to, inter alia, validate patient-specific optimized therapy in order to achieve desired cardiometabolic homeostasis, provide a cost-effective process for identification of vascular calcification and cardiovascular disease progression, and develop individualized interventions to slow the progression of vascular calcification in CKD/ESRD patients. Additionally, causality-based analysis modeling is developed to identify individual patient-specific drivers of vascular calcification. The causality analysis may include a physiology-based model of vascular calcification, the creation of a virtual patient population, and a generic virtual clinical trial environment for this and/or other renal pathophysiology.

In some embodiments, a vascular calcification model may be or may include a causal pathway-based physiological model that utilizes clinical data to identify patients and/or risk levels (e.g., high-risk patients) of progression of vascular calcification (VC) and cardiometabolic diseases to provide multifactorial intervention strategies targeting the risk factors. The vascular calcification models may use information including, without limitation, pulse pressure (PP, a proxy for pulse wave velocity) to parathyroid hormones (PTH), calcium (Ca), phosphate (PO₄), calcium-phosphate product (CaPO₄), neutrophil-lymphocyte ratio (NLR), and/or albumin (Alb). Pulse pressure may account for both cardiac and vascular conditions (e.g., atrial fibrillation, aortic insufficiency, arterial stiffness or arteriovenous malformation, aortic valve stenosis, cardiac insufficiency, or cardiac tamponade).

CKD/ESRD models, such as vascular calcification models, according to some embodiments may demonstrate the causal pathway of PTH, Ca, PO₄, NLR, and/or Alb on PP, and may demonstrate that there are likely paths from PTH, Ca, PO₄, CaPO₄, NLR to PP, where the strength of the relationships varies from patient to patient. Using these pathways, a dynamic model describing these interactions may be used to determine the impact of the dynamics on the progression of vascular calcification, and/or other conditions, and provide treatment recommendations and strategies to patients.

CKD/ESRD models and methods of using CKD/ESRD models may provide multiple technological advantages over conventional techniques, including advances in computing technology. In one non-limiting example of technological advantage, CKD/ESRD models may operate to accurately and efficiently model CKD/ESRD conditions in a manner not available using existing processes. In another non-limiting technological advantage, CKD/ESRD models may provide a causality analysis to identify patient-specific causes of CKD/ESRD conditions in patients and/or patient populations. CKD/ESRD condition risk, such as CVD risk, is multifactorial and interventions directed to a single risk factor are not effective. Accordingly, in one non-limiting technological advantage, CKD/ESRD condition analysis processes may determine multifactorial causes and interventions to CKD/ESRD conditions, such as CVD. A further non-limiting technological advantage may include providing a CKD/ESRD model configured to demonstrate a causal pathway of various conditions including, without limitation, PTH, Ca, PO₄, NLR, and/or Alb on PP, and determine paths from PTH, Ca, PO₄, CaPO₄, NLR, and/or Alb to PP.

Current computing systems, including machine learning approaches, are not able to provide causal and pathophysiological insights that are required for prescriptive analysis. In addition, the process of many CKD/ESRD conditions, such as vascular calcification, is not well understood, making the development of a mechanistic model (for instance, an “avatar” approach) impractical, if not impossible. Furthermore, differential temporal resolutions of clinical data may result in poor performance. Accordingly, another non-limiting technological advantage may include providing improved computing technology capable of computer-based determinations of CKD/ESRD conditions, such as vascular calcification, causation of CKD/ESRD conditions, and/or treatment recommendations for CKD/ESRD conditions (for instance, based on determined causation) that are efficient, accurate, and not currently available using conventional computer systems.

A person of skill in the art would recognize additional technological advantages based on the teachings of the present disclosure. Embodiments are not limited in this context.

Processes, techniques, methods, systems, and/or the like described in the present disclosure may be integrated into various practical applications. For example, CKD/ESRD condition analysis using processes and computational models according to some embodiments may determine one or more causal factors for CKD/ESRD conditions, such as vascular calcification, of a patient and/or patient population. In an additional example, CKD/ESRD condition analysis processes according to some embodiments may be integrated into the practical application of diagnosing a patient. In a further example, CKD/ESRD condition analysis processes according to some embodiments may be integrated into the practical application of administering treatment to a patient, such as providing treatment options, recommendations, prescriptions, and/or the like based on the patient information and a CKD/ESRD condition diagnosis and causation determination. For example, administration of a treatment may include determining a dosage of a drug, administering the dosage of a drug, determining a testing regimen, administering the testing regimen, determining a treatment regimen (such as a dialysis treatment regimen, parameters (for instance, ultrafiltration rate), or prescription), administering the treatment regimen, and/or the like. Embodiments are not limited in this context.

Additional technological advantages and integrations of embodiments into practical applications are described in, and would be known to those of skill in the art in view of, the present disclosure.

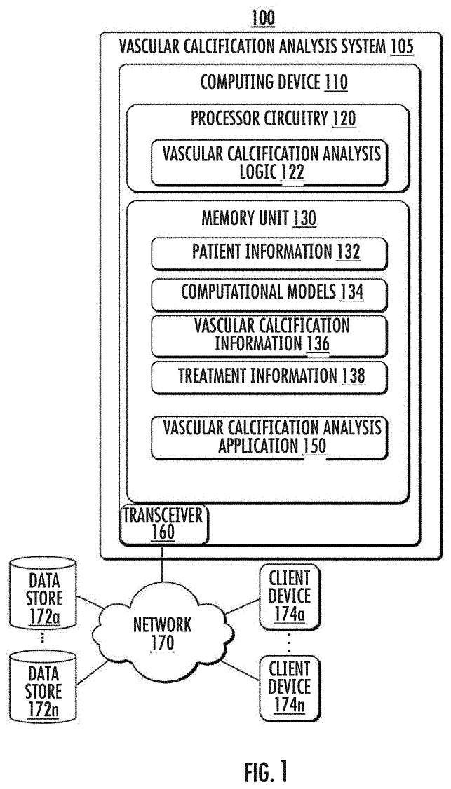

FIG. 1 illustrates an example of an operating environment 100 that may be representative of some embodiments. As shown in FIG. 1, operating environment 100 may include a CKD/ESRD condition analysis system 105. In various embodiments, CKD/ESRD condition analysis system 105 may include a computing device 110 communicatively coupled to network 170 via a transceiver 160. In some embodiments, computing device 110 may be a server computer or other type of computing device.

Computing device 110 may be configured to manage, among other things, operational aspects of a CKD/ESRD condition analysis process, such as a vascular calcification analysis process, according to some embodiments. Although only one computing device 110 is depicted in FIG. 1, embodiments are not so limited. In various embodiments, the functions, operations, configurations, data storage functions, applications, logic, and/or the like described with respect to computing device 110 may be performed by and/or stored in one or more other computing devices (not shown), for example, coupled to computing device 110 via network 170 (for instance, one or more of client devices 174a-n). A single computing device 110 is depicted for illustrative purposes only to simplify the figure. Embodiments are not limited in this context.

Computing device 110 may include a processor circuitry that may include and/or may access various logics for performing processes according to some embodiments. For instance, processor circuitry 120 may include and/or may access a CKD/ESRD analysis logic 122. In some embodiments, CKD/ESRD condition analysis logic 122 may operate to perform vascular calcification analysis processes according to some embodiments.

Processing circuitry 120, CKD/ESRD analysis logic 122, and/or portions thereof may be implemented in hardware, software, or a combination thereof. In some embodiments, CKD/ESRD analysis logic 122 may include and/or implement a dynamical system modeler process and/or a dynamical system learner process according to various embodiments. As used in this application, the terms “logic,” “component,” “layer,” “system,” “circuitry,” “decoder,” “encoder,” “control loop,” and/or “module” are intended to refer to a computer-related entity, either hardware, a combination of hardware and software, software, or software in execution, examples of which are provided by the exemplary computing architecture 1500. For example, a logic, circuitry, or a module may be and/or may include but are not limited to, a process running on a processor, a processor, a hard disk drive, multiple storage drives (of optical and/or magnetic storage medium), an object, an executable, a thread of execution, a program, a computer, hardware circuitry, integrated circuits, application-specific integrated circuits (ASIC), programmable logic devices (PLD), digital signal processors (DSP), field-programmable gate array (FPGA), a system-on-a-chip (SoC), memory units, logic gates, registers, semiconductor device, chips, microchips, chip sets, software components, programs, applications, firmware, software modules, computer code, a control loop, a computational model or application, an AI model or application, an ML model or application, a proportional-integral-derivative (PID) controller, variations thereof, combinations of any of the foregoing, and/or the like. In one non-limiting example, CKD/ESRD condition analysis logic 122 may include instructions loaded into processor circuitry (for instance, instructions or code associated with CKD/ESRD condition analysis application 150).

Although CKD/ESRD condition analysis logic 122 is depicted in FIG. 1 as being within processor circuitry 120, embodiments are not so limited. For example, CKD/ESRD condition analysis logic 122 and/or any component thereof may be located within an accelerator, a processor core, an interface, an individual processor die, implemented entirely as a software application (for instance, a CKD/ESRD condition analysis application 150) and/or the like.

Memory unit 130 may include various types of computer-readable storage media and/or systems in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid-state memory devices (e.g., USB memory, solid-state drives (SSD) and any other type of storage media suitable for storing information. In addition, memory unit 130 may include various types of computer-readable storage media in the form of one or more lower speed memory units, including an internal (or external) hard disk drive (HDD), a magnetic floppy disk drive (FDD), and an optical disk drive to read from or write to a removable optical disk (e.g., a CD-ROM or DVD), a solid-state drive (SSD), and/or the like.

Memory unit 130 may store various types of information and/or applications for a vascular calcification analysis process according to some embodiments. For example, memory unit 130 may store patient information 132, computational models 134, CKD/ESRD information 136, treatment information 138, and/or a CKD/ESRD condition analysis application 150. In some embodiments, some or all of patient information 132, computational models 134, CKD/ESRD information 136, treatment information 138, and/or a CKD/ESRD condition analysis application 150 may be stored in one or more data stores 172a-n accessible to computing device 110 via network 170. For example, one or more of data stores 172a-n may be or may include a HIS, an EMR system, a dialysis information system (DIS), an image archiving and communication system (PACS), a Centers for Medicare and Medicaid Services (CMS) database, U.S. Renal Data System (USRDS), a proprietary database, and/or the like.

Patient information 132 may include characteristics of a patient and/or a patient population that may be relevant to determining a CKD/ESRD condition, such as vascular calcification. In some embodiments, computational models 134 may be or may include one or more artificial intelligence (AI) models, machine learning (ML) models, deep learning (DL) models, neural networks (NN), Dynamical Systems (DS), combinations thereof, and/or the like.

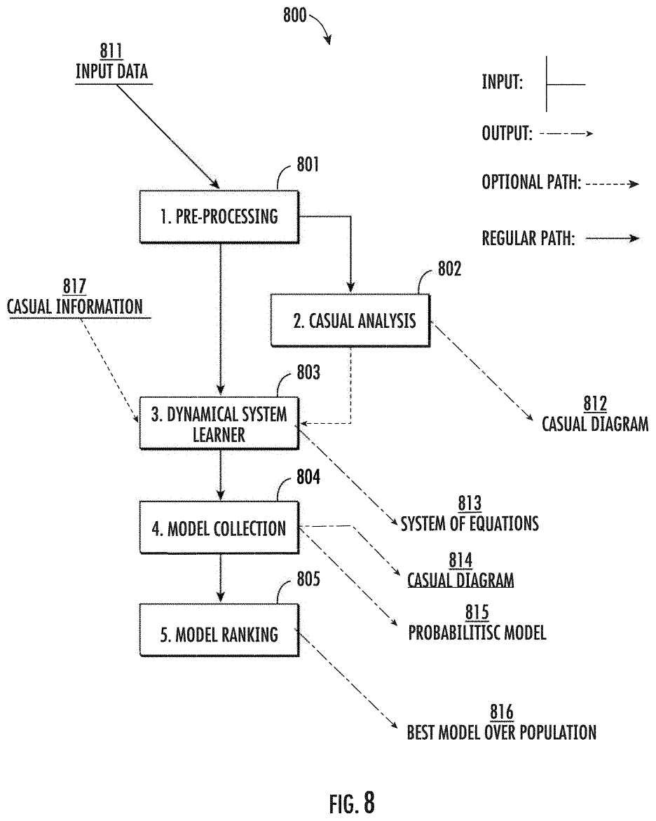

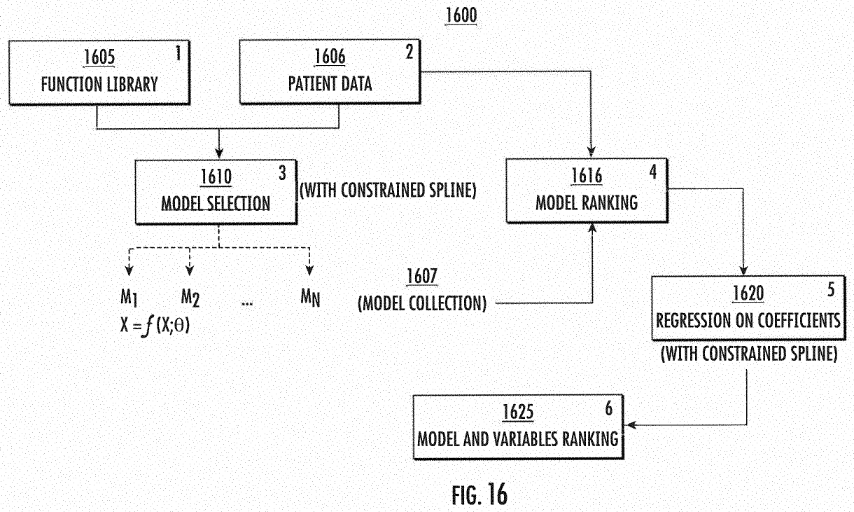

In some embodiments, CKD/ESRD condition analysis application 150 may use one or more computational models 134 to analyze patient information 132 to determine vascular calcification information 140 and/or treatment information 142. In general, calcification information 140 is information generated by a computational model 134 according to some embodiments to derive a vascular calcification diagnosis (for a particular patient and/or patient population, including causation, as described in the present disclosure. In some embodiments, CKD/ESRD condition analysis application 150 may include and/or implement a dynamical system modeler process and/or a dynamical system learner process according to various embodiments. In some embodiments, computational models 134 may include a dynamical system model generated via at least one of a dynamical system learner process (see, for example, FIG. 8) or a dynamical system modeler process (see, for example, FIG. 16).

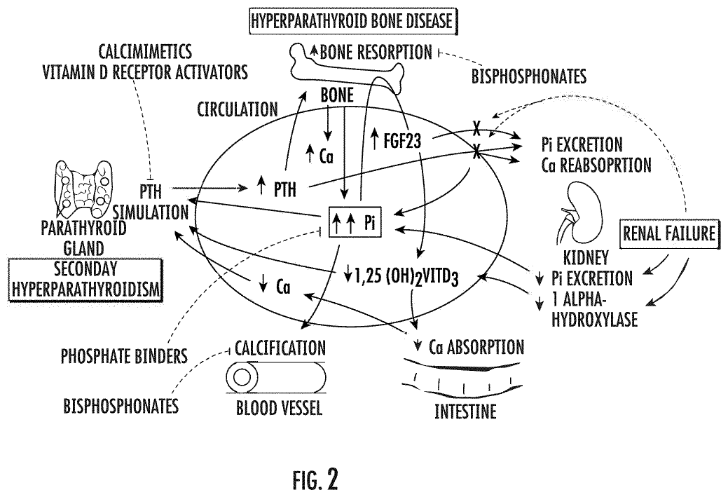

As demonstrated in FIG. 2, the processes and conditions of vascular calcification are complex and multifaceted (see Epomedicine. Complications of Long Term Dialysis [Internet]. Epomedicine; 2016 Apr. 19; available from: https://epomedicine.com/medical-students/complications-of-long-term-dialysis/). These conditions are complicated by CKD and other conditions. For example, CKD facilitates a pro-calcific environment to accelerate vascular calcification by reducing anti-calcific inhibitors (e.g., fetuin-A, Matrix Gla protein (MGP), and pyrophosphate (pyroPO4)) and uremic toxins. Vascular calcification is an active process that also involves osteo/chondrogenic trans-differentiation of vascular smooth muscle cells (VSMC) into osteoblast-like cells involving various interwoven signaling pathways.

Accordingly, some embodiments may provide a physiology-based model operative to, among other things, validate patient-specific optimized therapy to achieve desired cardiometabolic homeostasis, develop and validate a cost-effective algorithm for identification of vascular calcification and cardiovascular disease progression, and/or develop individualized interventions to slow the progression of vascular calcification in CKD/ESRD patients.

Although vascular calcification is used in examples, embodiments are not so limited. In particular, the analysis solutions according to some embodiments may be applied to CKD/ESRD conditions and/or pathophysiologies, for example, to develop a causality-based analysis model to identify individual patient-specific drivers of a condition. Non-limiting examples of CKD/ESRD conditions that may be modeled according to some embodiments may include anemia, bone mineral metabolism disorders, acid-base disorders, and/or the like.

CKD/ESRD condition analysis processes according to some embodiments may provide a causality analysis including, without limitation: a physiology-based model of a CKD/ESRD condition, such as vascular calcification; creation of a virtual patient population; generic virtual clinical (VC) trial environment (for instance, via software development and implementation); validation of CKD/ESRD condition models, such as vascular calcification models, for example, using data from ongoing clinical studies and/or using data from other collaborators.

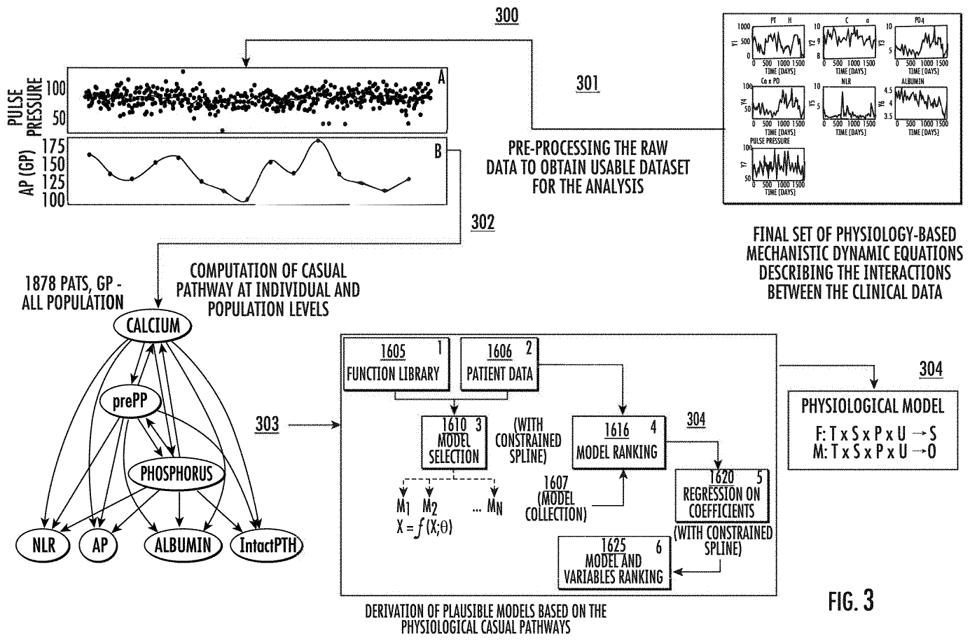

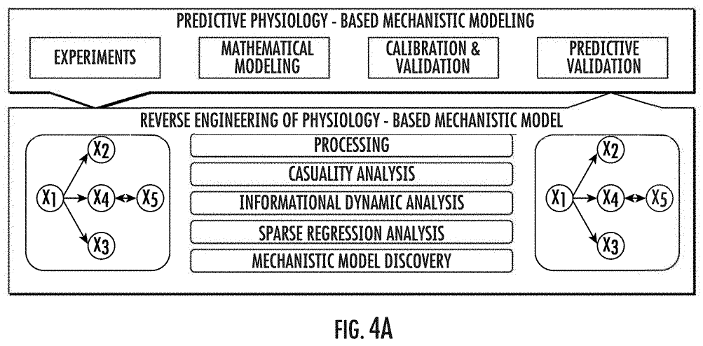

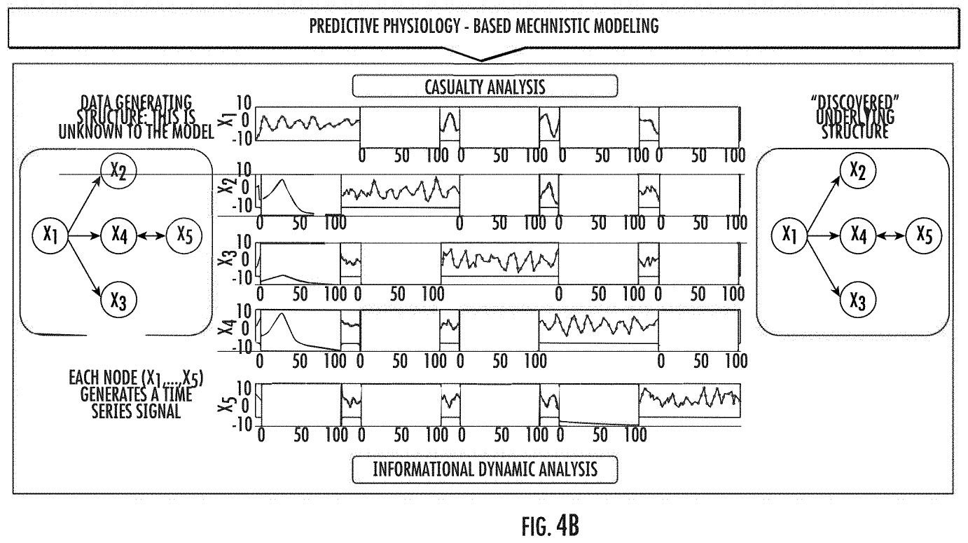

FIG. 3 depicts an illustrative and non-limiting CKD/ESRD condition analysis process according to some embodiments. Process 300 may include a dynamical system modeler process, such as process 304, (for instance, see FIG. 16). As shown in FIG. 3, a vascular calcification analysis process 300 may include pre-processing raw data to obtain a usable data set for analysis 301, computation of one or more causal pathways at an individual and/or population level 302, derivation of plausible models based on the physiological causal pathways 303, and/or determining a final set of physiology-based mechanistic dynamic equations describing the interactions between the data 304. FIGS. 4A and 4B depict illustrative and non-limiting examples of modeling approaches according to some embodiments.

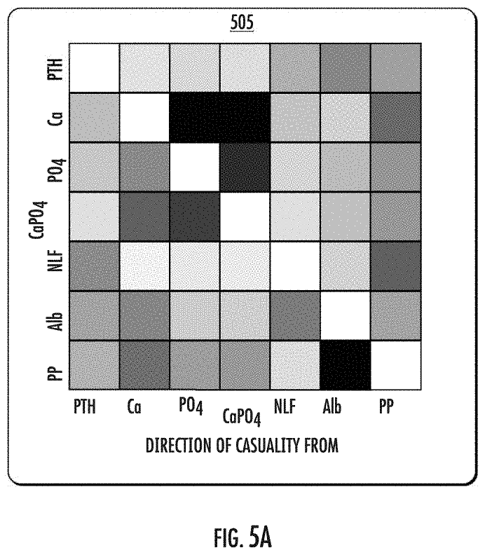

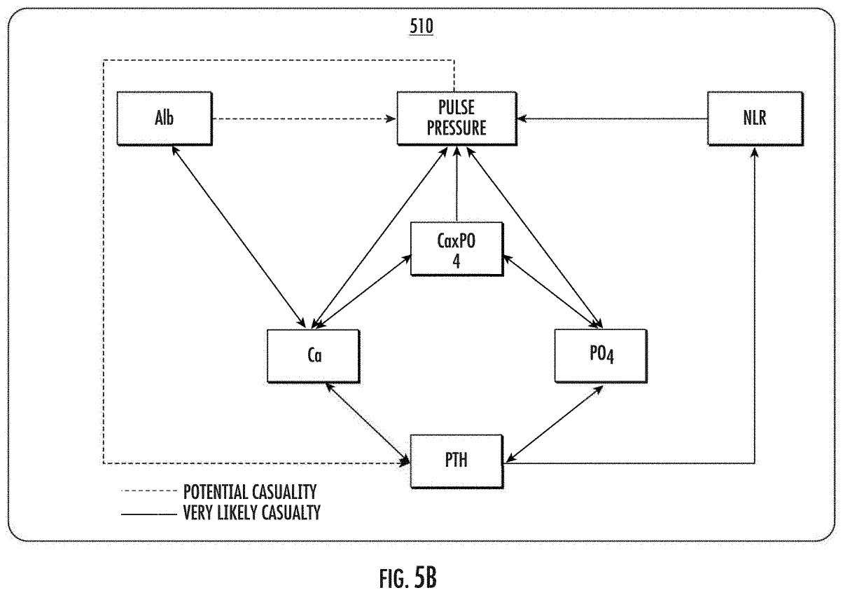

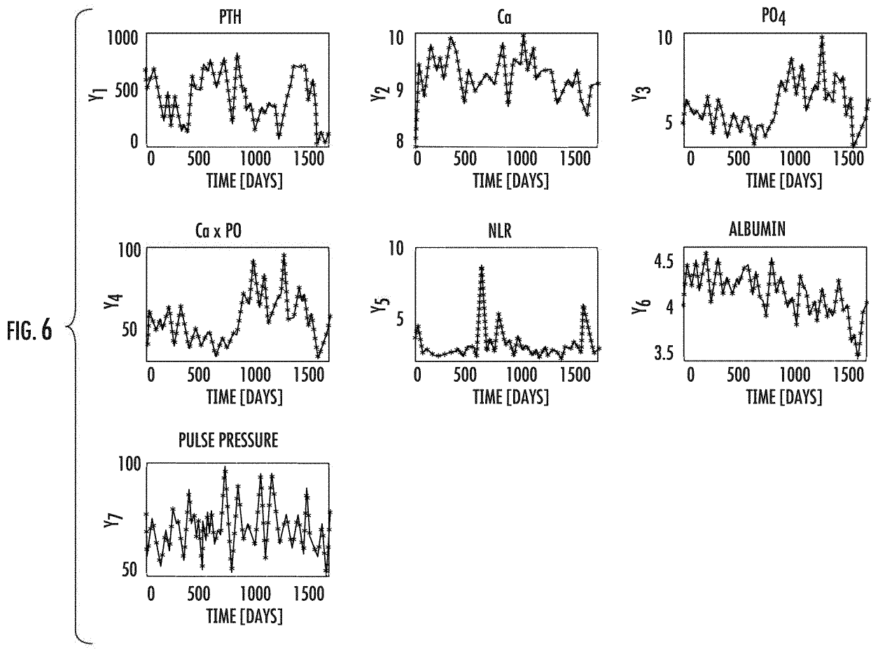

FIGS. 5A and 5B depict illustrative and non-limiting examples of causation output of a CKD/ESRD condition model according to some embodiments. FIG. 6 depicts illustrative clinical data used to determine the output shown in FIGS. 5A and 5B. As shown in FIG. 5A, a CKD/ESRD condition analysis process may generate a causality map or fingerprint 505 visually demonstrating potential causality. In general, the darker the color in a region (or square), the more likely a causal/directional link. As shown in FIG. 5B, a vascular calcification analysis process may generate a causality map or pathway map 510 visually demonstrating potential causality pathways for one or more CKD/ESRD condition indicators, for example, PP and/or pulse wave velocity for vascular calcification. A causality map or pathway map is a graphical representation of a possible causal relationship between two variables. A causal relationship is said to exist from variable X to variable Y if changes in the value of X lead to changes in the value of Y, from a dynamical system representation of the system that relationship is represented by a functional dependency of the time derivative of Y on X. Alternative definitions of causality can be stated in terms of time series modeling and forecasting, if a causal relationship exists from X to Y, then it is expected that the inclusion of past values of X in a forecast model of Y should improve the quality of the forecast.

There are various possible ways of extracting causal information from the collection a models determined according to some embodiment. Non-limiting examples of extraction methods may include probability-based causal analysis and ensemble causal analysis. Both methods rely on the same analysis on an individual level: y is said to cause x if the time derivative of x is an explicit function of y, i.e., \dot{x}=ƒ(y).

For the probability-baseds approach, to determine a single causal diagram for a population of patients or a collection of models consists of determining the probability that each individual term of the function library (Λ) is present in any arbitrary model in the collection. In other words, a probability model is constructed where for each element of the function library there is assigned the probability that the associated term is found in any model in the collection. A final model may be obtained by eliminating the terms in Λ with probabilities smaller than a given threshold. The causal diagram may then constructed by analyzing the surviving terms in this thresholded model.

For the ensemble causal approach, the ensemble causal diagram is built from the causal diagrams of each individual model added to the collection, i.e., for each new model added to the collection of models, a causal diagram is constructed. From the set of all causal diagrams in the collection a probability of occurrence may be assigned to each one of all the possible causal links between the different variables in the model (or for each element in the adjacency matrix). Finally, each link may be thresholded given its probability of occurrence in the whole collection of models.

A probability-based causal diagram may take into consideration each different term separately, when thresholding the terms in the final step it may select the highest probable model according to this approach, and extracts a causal diagram from this selected model. It is more sensitive to the heterogeneity of the models included in the collection and, as a consequence, the probability thresholds that need to be used are usually smaller. The thresholds may be configured according to various value systems, such as a range of 0 to 1 (or any other range). In some embodiments, the thresholds may include probability thresholds (for example, ranging between 0 to 1, 0% to 100%, and/or the like). Embodiments are not limited in this context.

An ensemble causal diagram extracts the causal diagram from each model in the collection and thresholds over the probability of each causal link. In that sense it is not sensitive to which particular terms were selected in each model, but only whether a given functional relationship between two variables existed. Therefore, it is expected that the causal relations obtained may be more robust, which is confirmed by the larger thresholds used when selecting the final causal diagram.

A vascular analysis process according to some embodiments may use the causation output to determine patients that are likely to have vascular calcification and/or causes of vascular calcification. Accordingly, vascular analysis processes may determine treatment recommendations based on the causation output that may be administered to target specific causes or potential causes of vascular calcification.

Some embodiments may use dynamical system representation of biological systems. A dynamical system may be understood as a system that changes in time and can be prescribed deterministically, i.e., there is a well-determined rule for its temporal evolution, in such a way that by knowing only this rule and the system's initial condition, it is possible to predict the system future states.

For example, some embodiments may operate to, given a set of noisy time-de data on multiple relevant variables, sampled from different realizations and on different times, construct a dynamical system model that represents the observed data and that allows the extraction of causal relations between the variables.

A large class of dynamical systems can be represented by a set of N differential equations in N variables represented by the state vector x=(x₁, . . . , x_{N}), with temporal evolution given by:

\dot{x}=ƒ(x;ξ),

where ξ is a vector of constant parameters and x is a vector of the state variables. Once a set of initial conditions is specified, i.e., x(t=0)=x₀ then the state of the system is known at any given future time t. For any given application, the state vector is composed of all the relevant variables to the description of the system, e.g., in the vascular calcification context its components can be pulse pressure, the concentration of calcium and phosphate in circulation, etc. The function ƒ encodes all the information of the relationship between the different variables in the system, and its mathematical structure reveals how the different system elements interact and influence each other. In some embodiments, the dynamical system model may be represented by a set of ordinary differential equations with or without time delay, and/or partial differential equations in 1, 2 or 3 spatial dimensions, or dependent of any other independent variable of relevance.

In some embodiments, a model may be defined by the vector function ƒ(x, ξ). An objective may be to find a function that represents all the multiple realizations of the system, differing only by the value of the parameters ξ between different realization; or to find different functions for different realizations but that allow forecasting the future value of the system state variables x based on its present value.

For many complicated and realistic applications, it is difficult, if not impossible, to determine the mathematical structure of ƒ from first principles. On the other hand, there has been an increase in the availability of data in many scientifical fields and major developments in machine learning tools designed to handle and analyze relevant data. For instance, there are data-oriented approaches to solve the problem of finding a mathematical representation of ƒ for complicated systems to which there are large data sets available.

In general, computational models according to some embodiments may operate to, given a set of noisy time-dependent data on multiple relevant variables, for example, sampled from different realizations and at different times, construct a dynamical system model that best represents the observed data and that allows the extraction of causal relations between the variables.

For noisy time-dependent data, the data may be represented by a set of observed variables yᵢ, where i=1, . . . , M and M is the number of state variables, the relationship to the observables y and the actual state variables x is given by:

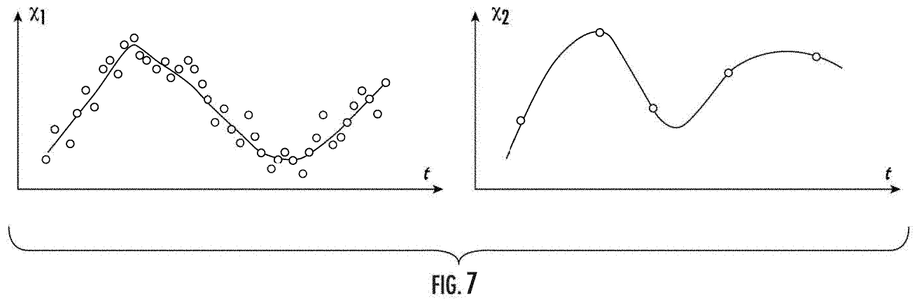

yᵢ(t)=g(xᵢ(t))+ [symbol] (0,σᵢ),

where the second term represents a normally distributed noise with zero mean and standard deviation σᵢ; and g(⋅) can be any function leading x(t) to y(t) (even the identity function, in which case the observable and the state variable are the same, except for the observational noise). In some embodiments, each variable xᵢ has a different degree of observational noise, represented by a different standard deviation σᵢ. A schematic representation of the data is provided in FIG. 7, in which the points represent the observed data of two state variables x₁ and x₂, the lines represent the true underlying dynamics without noise.

In some embodiments, realization may provide that the data set available is supposed to represent the system in different realizations, i.e., there are data available of the system in different experimental or observational conditions. For observational studies that can mean data on different patients with similar conditions; for experimental studies different repetitions of the same experiments that generates the data. The underlying assumption is that every realization of the system can be described by the same model, represented by the function ƒ that provides the dynamical evolution of the system. Different realizations are not expected to share the same set of parameters ξ.

Some embodiments may perform time sampling, e.g., in which the observables of the system are not expected to be sampled at the same times, i.e., for each observable and each state variable there is a different time grid (t₁, t₂, . . . , tₗ, . . . , t_{N}), where N is the total number of observations for a given observable and it might be different in different realizations of the system. Also, the time step for a given realization of the system does not need to be uniform, i.e., tᵢ₊₁−tᵢ≠tⱼ₊₁−tⱼ for i≠j.

Accordingly, in some embodiments, from the extracted model, it is possible to establish causal relationships between the state variables of the system, for instance, by looking at the arguments of a given component of the function ƒ. In this context causality should be interpreted as: if X is said to cause Y, then it is expected that a change in the value of X leads to a change in the value of Y, in the dynamical system representation of the system that reads \dot{Y}=ƒ(X, ξ).

FIG. 8 illustrates an illustrative CKD/ESRD condition analysis processes according to a first embodiment in accordance with the present disclosure. For example, FIG. 8 depicts a dynamical system learner process for determining and ranking models according to some embodiments. As shown in FIG. 8, a dynamical system learner process 800 may include pre-processing 801. In some embodiments, pre-processing 801 may include transforming input data 811 into one or more formats, for example, a format appropriate to code, data structures, and/or the like.

In various embodiments, causal analysis 802 may correspond to extracting causal information from input data 811, which may be outputted in various forms, such as the format of a causal diagram 812.

Important insight may be obtained from data by analyzing how much information is exchanged between different variables, such analysis can help establish cause-effect relationships for the state variables of the system. A traditional approach is based on the Granger causality, a linear method that states that Y “Granger causes” X if the inclusion of Y in a predictive model for X increases the accuracy of such a model (see, for example, Granger, C. W. J., ‘Investigating Causal Relations by Econometric Models and Cross-Spectral Methods’, Essays in Econometrics vol II: Collected Papers of Clive W J. Granger, 37(3), pp. 31-47 (2008)). Although useful in many contexts, Granger causality assumes linearity and stationarity, both assumptions cannot be assured in medical and biological applications. For that reason, several generalizations of the Granger procedure have been proposed, in particular, a kernel-based version of it was developed specifically to deal with nonlinear systems (see, for example, Marinazzo et al., ‘Kernel-Granger causality and the analysis of dynamical networks’, Physical Review E—Statistical, Nonlinear, and Soft Matter Physics, 77(5), pp. 1-9 (2008)).

An advantage of employing such causality methods based on time series forecasting lies on their relative simplicity and low computational cost, allowing fast results that can help understand complex relationships between variables. Such methods may require the time series to be sampled in time with enough precision so that the underlying models have sufficient predictive power. If that condition is not satisfied, imputation and modeling of the time series may be used to provide enough precision. The result, in the form of a causal diagram, is dependent on the imputation used; however, it may still provide meaningful information about the topological structure of the causal diagram of the system.

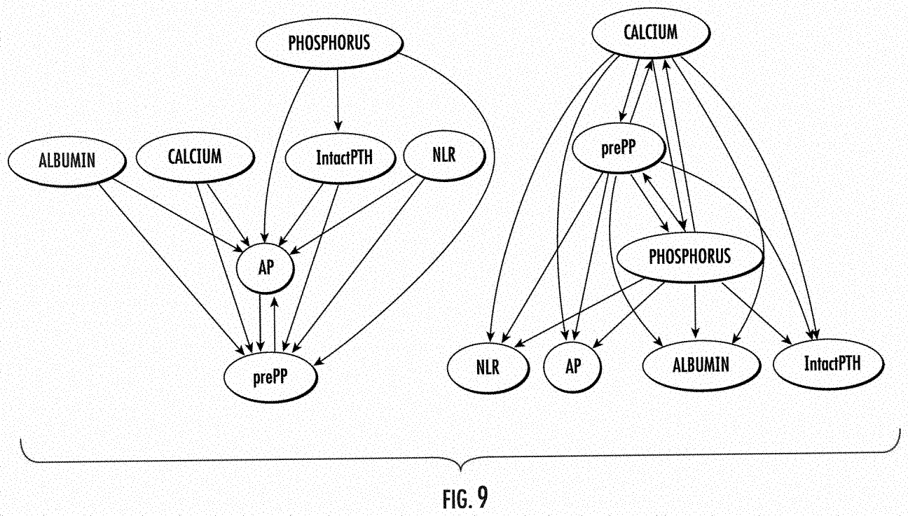

FIG. 9 illustrates exemplary causal diagrams according to the present disclosure. More specifically, FIG. 9 depicts two causal diagrams obtained by the kernel Granger algorithm when applied to the time-series generated from data by two different imputation methods: Average over GP and KNN 901 and GP only 902. Although providing different results, there are some topological similarities (if causal directionality is ignored) between diagrams 901 and 902, suggesting at least that some of the observed causal relationships (or their absence) might be meaningful. Also, these diagrams should not be analyzed independently of the current biological understanding of the mechanisms leading to vascular calcification.

In some embodiments, a dynamical system learner step 803 may operate to build a symbolic dynamical system model. In various embodiments, dynamical system learner step 803 may receive causal information 817 as input. From running one or more of previous steps 801-803 in a set of data from multiple patients, it may be possible to extract a collection of models in a model collection 804 step. By analyzing the frequency of occurrence of specific terms in such collection, embodiments may build a probabilistic representation for the system of equations 813, from the dependencies of the different models extracted it is possible to build a causal diagram 814 and/or probabilistic model 815, for example, for a patient or an entire population of patients.

In some embodiments, a model ranking 805 step may operate to calculate a model rank, for example, determining one or more optimal models 816, for instance, indicating which model(s) performs better when explaining the dynamical relationships between the variables in the input data.

In various embodiments, input data to a CKD/ESRD condition analysis processes, such as dynamical system learner process 800, may be or may include a set of noisy time series with missing values. In general, a time series may correspond to measurements of a variable of interest from patient physiology made at different moments in time. Assume y is such variable (e.g., pulse pressure or concentration of calcium in the patient blood stream), a time series for y corresponds to a set of N measurements of y made at the times t₁, t₂, . . . , t_{N}, and is denoted: {y}={y₁, y₂, . . . y_{N}}.

Each observable measured at time t is described by its value y(t); however, to measure the true value of the underlying state variable x directly and precisely is impossible. Consequently, the measurement provides a value y(t) called an observation of x(t), which relates to the actual value of x through the following equation:

y(t)=g(x(t))+ [symbol] (0,σ).

For example, it may be assumed that each value of y is a function of the corresponding value of x(g(⋅)) can be the identity function, in which case y corresponds to the state variable x, except for the noise), and an additive Gaussian noise with zero mean and standard deviation σ. The added noise may have a different distribution other than Gaussian, the distribution can be for example, but not limited to, student-t distributed noise, or lognormal noise, or exponentially distributed noise.

The presence of missing values, which may be represented by NA, means that at any particular time t the corresponding value y(t) was not measured and is therefore missing. For a set of noisy time series with missing values, since more than one observable are measured simultaneously, there is not one but M different variables. Each time series may be denoted {y}ⱼ where j=1, . . . , M.

Representing all this information together, the input can be written as a matrix where each column corresponds to the time series of a different observable, and each row corresponds to a different measurement in time. Therefore, the full input Y can be represented as:

$$Y = ( \begin{matrix}{y}_{1 , 1} & {y}_{1 , 2} & … & {y}_{1 , M} \\ {y}_{2 , 1} & NA & … & {y}_{2 , M} \\ ⋮ & ⋮ & ⋱ & ⋮ \\ NA & {y}_{N , 2} & … & {y}_{N , M}\end{matrix} ) ,$$

where the missing values are placed randomly for illustrative purposes. Not all the variables have the same missing value rate, i.e., some variables have more missing values than others and at different times. The same is valid for the amplitude of the noise, some variables have higher noise to signal ratio than others, in other words, the standard deviation σⱼ of the additive noise may not be the same for all {y}ⱼ.

In some embodiments, input causal information (such as causal information 817) may be in a form based on one of the following definitions of causality:
1. The variable yᵢ is said to cause yⱼ, if changes in the value of yᵢ lead to direct changes in the value of yⱼ. In other words, the time derivative of yⱼ is an explicit function of yᵢ, i.e., \dot{y}ⱼ=ƒ(yᵢ) (“direct causality”);
2. The variable yᵢ is said to cause yⱼ, if including yᵢ in an explanatory model for yⱼ improves the accuracy of such model, i.e., by including information on the past values of yᵢ we improve our ability to predict values of yⱼ. Mathematically, this can be represented as: yⱼ(t)=h(yᵢ(t−τ)) where h(⋅) is the appropriate modelling function and τ is a time delay (“explanatory model causality”).
   In some embodiments, causal information obtained from expert knowledge on the subject being studied can be used as input to the process. Such information can be represented in the form of a binary matrix of T and F: if the element of i-th row and j-th column is T, then yᵢ is allowed to cause yⱼ; opposed to the situation in which such element is F, in which case it is known that yᵢ does not cause yⱼ. For example, if it is known, from physiological reasons, that yᵢ does not cause yⱼ, then the corresponding element should be marked F. If we know nothing about their relationship, then the corresponding element should be marked T.

Some embodiments may perform time series pre-processing. For example, a first approach to handle the missing values may be to impute them, following some established criteria. In most applications, standard imputation schemes usually involve replacing the missing values by some measure of the variable distribution, usually by the mean or median, be it from the entire time series or from the first neighbors of the missing point.

However, if the missing values outnumber the observations for a given variable, these standard imputation schemes will lead to a biased result, in which case more robust techniques would need to be employed. In such a scenario, the imputation acquires a modeling characteristic, in which the missing values are replaced by a function that satisfies few conditions imposed by the researcher (such as continuity) and that are consistent with the observed data. The advantage of such approach is the possibility of applying time-series analysis to the imputed data, such as Granger-based causality measures and their various potential modifications.

As shown in FIG. 8, pre-processing 801 may be a first step in the CKD/ESRD condition analysis processes 800. A goal of pre-processing may be to read the raw data and transform it into a format that can be dealt with by the subsequent steps. In some embodiments, pre-processing aims to (i) minimize the effect of the noise in the time series; and (ii) impute the missing values of the raw data as to transform the data into an uniform time series representation.

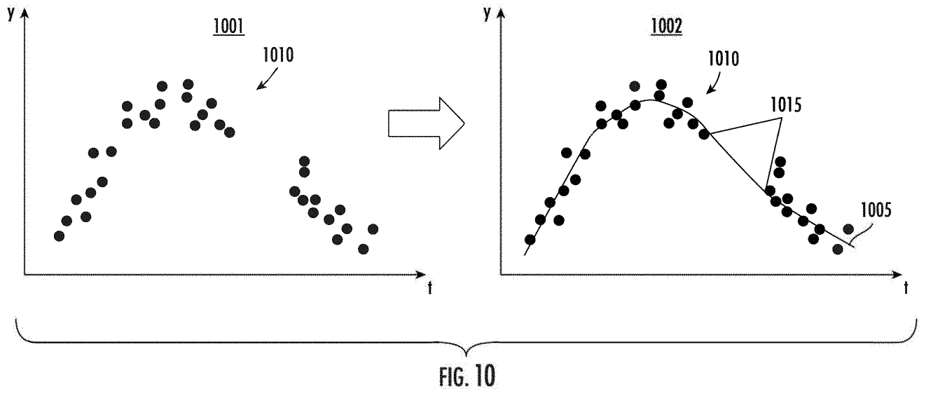

FIG. 10 illustrates exemplary pre-processing according to the present disclosure. Graph 1001 depicts the raw or original data points 1010 and graph 1002 depicts the original data points with a line 1005 that corresponds to the pre-processing. In general, line 1005 is smoother than points 1010, meaning it was able to capture the underlying dynamics reducing the effect of noise. Also, at the section in which there are missing values in the original data, the pre-processing imputed meaningful values (as depicted by segment 1015 in graph 1002).

In some embodiments, pre-processing may operate to perform one or more of: transform input data into a uniform representation for all variables; smoothen the data and reduce the importance of noise, trying to extract the relevant dynamical evolution of the variables; impute missing values with significant values that approximate the underlying true value of the variable; and increase the number of points available to feed the code, in order to improve performance.

Various processes, methods, algorithms, and/or the like may be used for pre-processing according to some embodiments. Non-limiting examples of methods may include spline, smoothing spline, and/or Gaussian processes.

In general, the Gaussian Process (GP) may operate to generalize the concept of a Gaussian probability distribution for random numbers for the case of stochastic processes. Each value ƒ(x) of the process is taken from a Gaussian distribution with statistical properties given by a covariance matrix K, that determines how the point x covaries with previous observations xᵢ. The covariance matrix is calculated on a function of the distance between the points x and xᵢ, called the kernel. The obtained result is dependent of the choice of the kernel function. A general kernel function (which is appropriate when not much is known about the underlying process generating the observed data) is the Radial Basis Function kernel (or RBF) which, given two observations x and x′, may be defined as:

$$K ( x , {x}^{′} ) = exp ( - \frac{{‖ x - {x}^{′} ‖}^{2}}{2 {σ}^{2}} ) .$$

This kernel is infinitely differentiable which leads to a smooth Gaussian process over the observed data, making it suitable to impute missing data in observations from natural processes. On the downside, for extremely under sampled observations, in which case the distance between successive points is too large, so that K(x, x′)≈0, Gaussian processes may fail to appropriately estimate the underlying dynamics of the system. Gaussian processes also effectively handle noisy data by adding a constant value α to the diagonal of the covariance matrix.

Another suitable kernel is the Matérn covariance function Kᵥ(\vec{t}, \vec{t}′), given by:

$${K}_{v} ( \vec{t} , {\vec{t}}^{′} ) = \frac{{2}^{1 - v}}{Γ ( v )} {( \frac{\sqrt{2 v} r}{ℓ} )}^{v} {J}_{v} ( \frac{\sqrt{2 v} r}{ℓ} ) ,$$

where [symbol] is a typical length scale, Γ(⋅) is the Gamma function, Jᵥ(⋅) is the modified Bessel function of the second kind, r=|t−t′| is the distance between the two arguments of the kernel and v is a positive parameter, such that the process η(x) is k-times mean-square differentiable if and only if v>k. A common value is v=3/2, so only once differentiability is required for the noisy data. Any other kernel function of the distance between two points in the time series can be used in Gaussian Process pre-processing, and it should not be limited to the previous two examples.

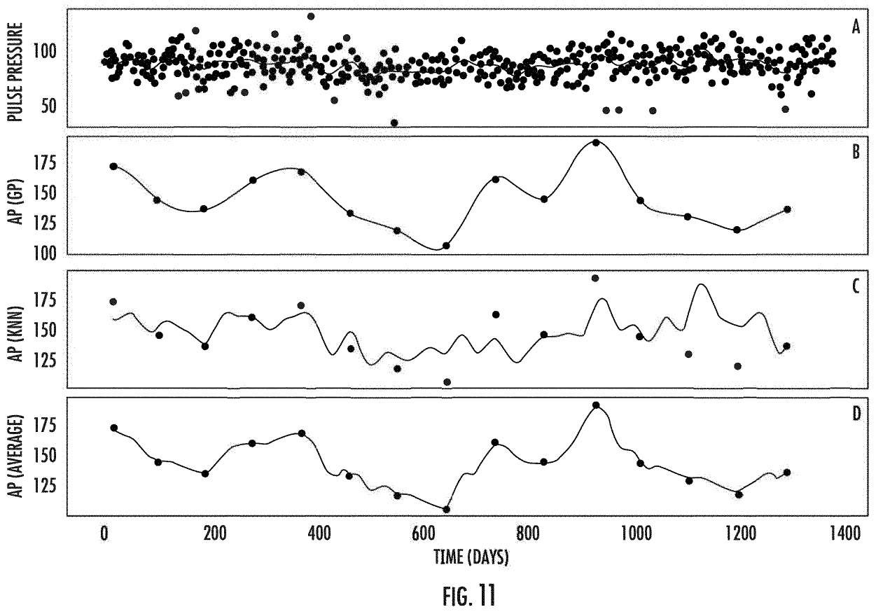

FIG. 11 depicts illustrative Gaussian processes according to some embodiments. In graph A, the pulse pressure is modeled by a noisy Gaussian process, in this way the data is smoothed and it is possible to capture the trend of the variable over time, instead of its local noisy fluctuation. In graph B, the data for Alkaline Phosphatase (AP) is displayed, the full line represents the Gaussian process. In this case, the GP is exact over the data points and it imputes the intermediate values with a smooth function.

To assume a dynamical system representation implies, among other things, that the state variables of the system are connected through a mathematical representation of their temporal evolution. Because of that, similar states are expected to generate similar outcomes. It is possible to use this reasoning in order to impute the missing values of the data using the nearest neighbor algorithm.

In one example, the state of the system may be characterized as a vector \vec{y}(t) with missing values which is sampled at times t₁, t₂, . . . , t_{L}. If the value of the i-th component of the state vector at time tⱼ is missing, i.e., yᵢ(tⱼ)=NA, then its value is replaced by:

yᵢ(tⱼ)= [symbol] k nearest neighbors i-th component [symbol] ,

where the angled bracket represents the average, and the “k nearest neighbors” (k-NN or KNN) are the k different points \vec{y}(tₖ) with tₖ≠tⱼ closest to \vec{y}(tⱼ), where distances are calculated only with the components of \vec{y} that form the orthogonal complement of the missing value.

Because only the missing values are imputed, the obtained time series is discontinuous. A Savitzky-Golay filter may be applied to smoothen the result, or any other smoothing filter technique. In graph C of FIG. 11, the KNN imputation is represented for the same data as in graph B. Because the final signal is smoothed, it does not have the exact value at the data points; instead, it only captures an overall trend of fluctuations of the time series under the assumption that all state variables are connected and that similar states lead to similar dynamics.

The two imputation methods presented so far can capture different features of the underlying dynamical system: Gaussian processes favor precision at the data points; and KNN favors similarity of recurrent states. In order to leverage both advantages, it is possible to calculate an average of them, if each point and method is carefully weighted, the resulting signal can be smooth and representative of the dynamics.

The resulting averaged time series xᵢₘₚ(t) may be calculated as:

$${x}_{imp} ( t ) = \frac{1}{σ ( t )} {\sum }_{i = 1}^{{N}_{P}} {w}_{i} ( t ) {x}_{i} ( t ) ,$$

where wᵢ(t) is the contribution at time t of each of the N_{P} points in the data set, and σ(t)=Σᵢ₌₁^{N}^{_{P}}wᵢ(t) is a normalization factor. The weight of each data point may be given by:

$${w}_{i} ( t ) = [ {e}^{- \frac{{( t - {t}_{i} )}^{2}}{2 {τ}^{2}}} + \frac{ρ}{{N}_{P}} ] ,$$

where 0≤ρ≤1 is a regularization factor and τ is a typical time scale. The contribution at time t of each data point to the average may be given by:

$${x}_{i} ( t ) = \frac{1}{{σ}_{i} ( t )} {\sum }_{α} {ω}_{α , i} ( t ) {x}^{( α )} ( t ) ,$$

where the Greek index iterates over the different imputation methods; ω_{α}(t) are the weights of each method and may be given by:

ω_{α,i}(t)=Π_{β≠α}dᵢ^{(β)}(t),

with the following:

$${d}_{i}^{( β )} ( t ) = \sqrt{{( \frac{t - {t}_{i}}{Δ t} )}^{2} + {( \frac{{x}^{( β )} ( t ) - {x}_{i}}{Δ x} )}^{2} ,}$$

where Δt=max(tᵢ)−min(tᵢ) and Δx=max(xᵢ)−min(xᵢ) for all i. And σᵢ(t)=Σ_{α}ω_{α,i}(t) is the normalization factor. In other words, the weights were carefully chosen to favor the imputation that is closer to the data points.

In FIG. 11, graph D represents the weighted average of the signals of graphs B and C. The resulting time series successfully represents the available time series and captures local fluctuations from the nearest neighbors' imputation.

In some embodiments, imputation methods may use machine learning (ML), artificial intelligence (AI), and/or deep learning (DL) techniques. Non-limiting examples of ML may include recursive neural networks (RNN), cognitive neural network (CNN), and generative adversarial networks (GAN), or a combination and/or adaptation of both. The RNN architecture is suitable for time series analysis as it includes information about previous values when training the network; as for the GAN's, this architecture is very efficient in generating fake data due to its use of two competing networks: one attempts to model a mapping from noise to the true data, and the other one reads the data and assigns to it a probability of it being true. The combined iteration of these two adversarial networks eventually converge to a good quality of the generated fake data.

In some embodiments, a causal analysis may be or may correspond with causal analysis 802 of process 800 of FIG. 8. A causal analysis may operate to infer underlying and robust causal relationships between the variables. In some embodiments, a causal analysis may involve “explanatory model causality,” for example: The variable yᵢ is said to cause yⱼ, if including yᵢ in an explanatory model for yⱼ improves the accuracy of such model, i.e., by including information on the past values of yᵢ we improve our ability to predict values of yⱼ. Mathematically, this can be represented as: yⱼ(t)=h(yᵢ(t−τ)) where h(⋅) is the appropriate modelling function and τ is a time delay.

Causal analysis may operate to provide insight about the possible mechanistic relationship between the observables of the system and the underlying state variables. In addition, causal analysis may operate to provide a causal diagram that may be used as input to a dynamical system learner (e.g., dynamical system learner 803 of FIG. 8), reducing the necessary terms in the library of functions, which reduces the time of calculation.

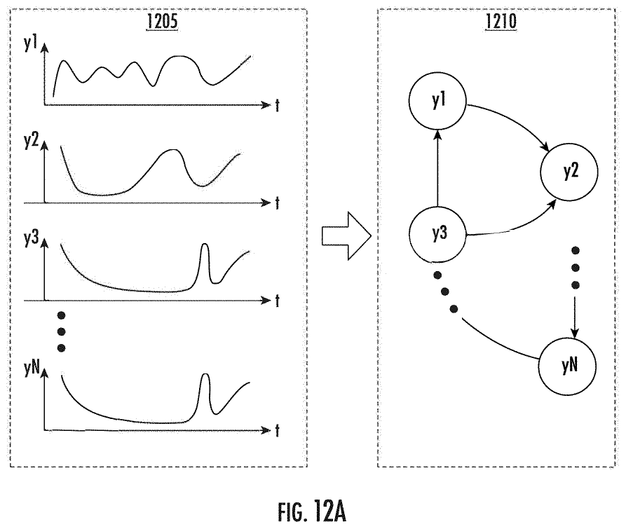

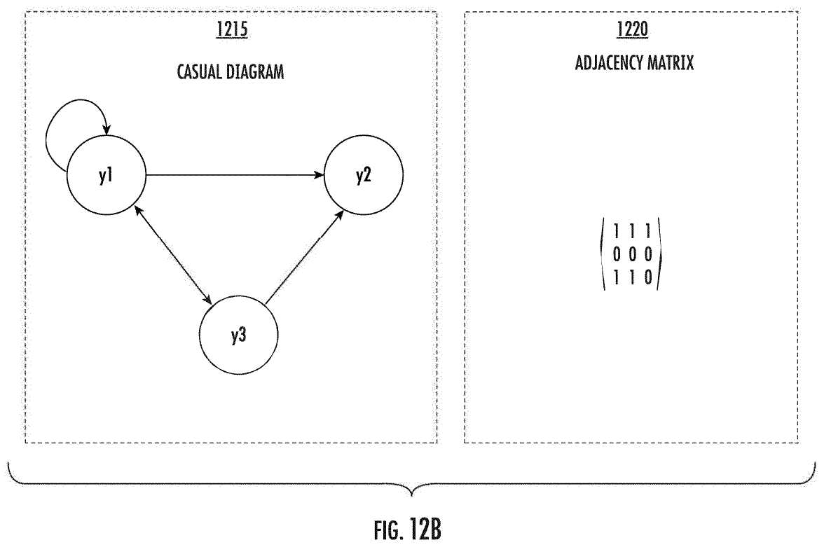

FIG. 12A illustrates an example of a causality analysis in accordance with the present disclosure. As shown in FIG. 12A, as input 1205, the system may receive a set with the time series of the observables. Although input 1205 is depicted as a set of graphs, input 1205 may have various other forms, such as raw data, matrix, table, csv data, text file, and/or the like. The output 1210 of the system may be causality information. In some embodiments, the causality information may be or may include a causality diagram or graph, which is a graph where each node represents one of the input observables, and the nodes may be connected by directional links or arrows demonstrating causality. For example, if there is an arrow connecting the node yᵢ to the node yⱼ, then yᵢ is said to cause yⱼ.

The causality diagram may have other forms, such as a matrix form, called an adjacency matrix. This is a binary matrix where if the element in the i-th rom and j-th column is 1 (or True) it means that yᵢ causes yⱼ. FIG. 12B illustrates an example of a causality matrix in accordance with the present disclosure. As shown in FIG. 12B, matrix 1220 is a 3-dimensional adjacency matrix for causal diagram 1215. The first row of adjacency matrix 1220 has three ones, meaning that y₁ causes y₁, y₂ and y₃, which can be confirmed at causal diagram 1215 by the arrows leaving y₁. As no arrow leaves y₂, y₂ does not cause any other variable, then the second row of adjacency matrix 1220 is zero. As for y₃, it only causes y₁ and y₂, but not itself, therefore the third row of adjacency matrix 1220 has non-zero elements only in the first two columns.

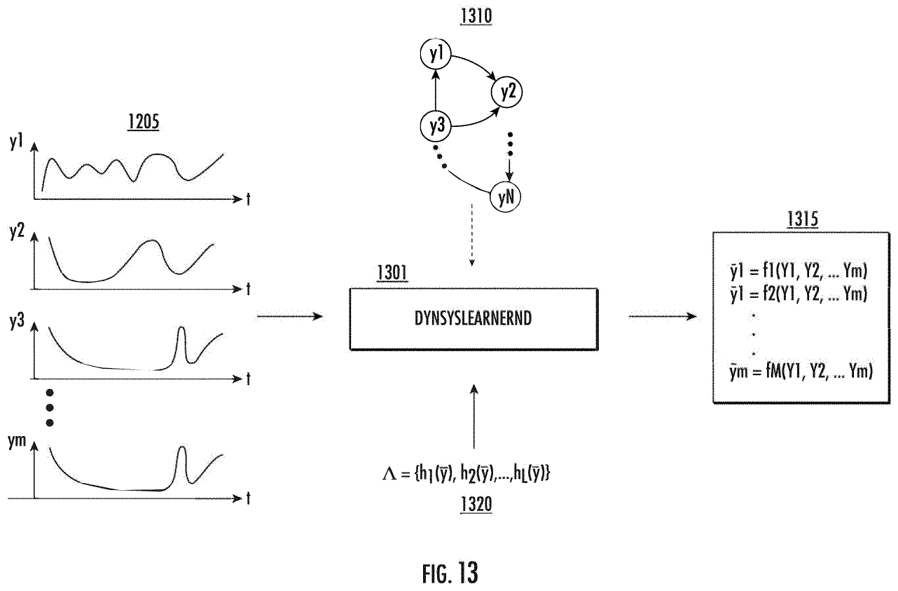

FIG. 13 illustrates an exemplary dynamical system learner in accordance with the present disclosure. As shown in FIG. 13, a dynamical system learner (or “DynSysLearner” and/or “DynSysLearnerND”) may receive various inputs. Non-limiting examples of inputs may include a time series 1305 for the pre-processed system observables: y₁(t), y₂(t), . . . , y_{M}(t), a library of functions Λ 1320, and/or causality information 1310, such as a causal diagram, a causality adjacency matrix, and/or the like. In various embodiments, DynSysLearner 1301 may generate output 1315, including a system of ordinary differential equations that describe the time evolution of the input variables yᵢ(t). For example, DynSysLearner 1301 may find a symbolic representation of the functions ƒ₁(t), ƒ₂(t), . . . , ƒ_{M}(t), such as:

\dot{y}ᵢ=ƒᵢ(y,t) i=1, . . . ,M.

A dynamic system learner, such as dynamic system learner 1301, may perform various technical functions, including, without limitation: from time series data, infer a set of ordinary differential equations, or partial differential equations, or time delay differential equations, that better explain the time evolution of the observed variables; obtain a different model, with either different equations or only different parameters, for each one of the different realizations of the data; an obtained set of equations may be able to be used to make forecasts about the system state; an obtained set of equations may be used to model the effect of treatments for the patient.

In various embodiments, a dynamic system learner may include and/or use various processes, functions, algorithms, and/or the like. For example, an algorithm may be configured to extract dynamical system models from data. The algorithm may be configured to find the set of M functions ƒᵢ, such as:

$${\dot{y}}_{i} = {f}_{i} ( \vec{y} , t ) = \frac{N ( y , t )}{D ( y , t )} i = 1 , … , M ,$$

which can be rewritten as:

D(y,t)\dot{y}ᵢ−N(y,t)=0.

A method to solve this problem may include writing this expression as a product between a matrix (Θ) built from a library of functions (Λᵢ), and the input data (Y and \dot{Y}), and a vector of coefficients \vec{ξ}ᵢ, such as:

D(y,t)\dot{y}ᵢ−N(y,t)=Θ(Λ,Y,\dot{Y}ᵢ)\vec{ξ}ᵢ=0,

where the preprocessed time series Y is given by:

$$Y = ( \begin{matrix}\vert & \vert & & \vert \\ {y}_{1} & {y}_{2} & … & {y}_{M} \\ \vert & \vert & & \vert\end{matrix} ) = ( \begin{matrix}{y}_{1} ( {t}_{1} ) & {y}_{2} ( {t}_{1} ) & … & {y}_{M} ( {t}_{1} ) \\ {y}_{1} ( {t}_{2} ) & {y}_{2} ( {t}_{2} ) & … & {y}_{M} ( {t}_{2} ) \\ ⋮ & ⋮ & ⋱ & ⋮ \\ {y}_{1} ( {t}_{N} ) & {y}_{2} ( {t}_{N} ) & … & {y}_{M} ( {t}_{N} )\end{matrix} ) ,$$

and
where M is the number of dimensions of the input data, and N is the number of times in which it was sampled.

In some embodiments, a library of functions, Λᵢ={h₁(\vec{y}), . . . , h_{L}(\vec{y})} may include a set of functions of the input \vec{y}. By applying the library of functions to the input data:

$${Λ}_{i} ( Y ) = ( \begin{matrix}\vert & \vert & & \vert \\ {h}_{1} ( Y ) & {h}_{2} ( Y ) & … & {h}_{L} ( Y ) \\ \vert & \vert & & \vert\end{matrix} ) ,$$

In which the algorithm finds M sparse sets of coefficients \vec{ξ}ᵢ={ξ₁, . . . , ξ [symbol] }, such as:

Θ(Λ,Y,\dot{Y}ᵢ)\vec{ξ}ᵢ=(Λ(Y)∘\vec{\dot{Y}}ᵢ−Λ(Y))\vec{ξ}ᵢ=0 i=1, . . . ,M,

where ∘ is a row-wise multiplication:

$$Λ ∘ \vec{y} = ( \begin{matrix}{λ}_{11} {y}_{1} & {λ}_{12} {y}_{1} & … & {λ}_{1 L} {y}_{1} \\ {λ}_{21} {y}_{2} & {λ}_{22} {y}_{2} & … & {λ}_{2 L} {y}_{2} \\ ⋮ & ⋮ & ⋱ & ⋮ \\ {λ}_{N 1} {y}_{N} & {λ}_{N 2} {y}_{N} & … & {λ}_{NL} {y}_{N}\end{matrix} ) .$$

In some embodiments, a causality adjacency matrix ( [symbol] ) may be a M×M binary matrix, if [symbol] ᵢⱼ is 1, it means that yᵢ causes yⱼ; zero otherwise. Its effect on the code is to filter out columns of the library of functions Λᵢ (the index refers to the M different libraries used to each variable yᵢ). Then, if the [symbol] -th function of the library is an explicit function of yⱼ, i.e., if [symbol] = [symbol] (yⱼ); and Cᵢⱼ=0, then [symbol] is removed from Λᵢ.

To solve the equation Θξᵢ=0, the sequentially thresholding least squares (STLSq) algorithm may be used. The method relies on transforming the problem to a convex problem, by removing one of the columns of the matrix Θ and rearranging the problem:

θⱼ=Θⱼ(Λᵢ,Y,\dot{Y}ᵢ)\vec{ξ}ᵢ^{(j)},

where θⱼ is the j-th column of the matrix Θ(Λᵢ, Y, \dot{Y}ᵢ), and Θⱼ corresponds to Θ with the j-th column removed. \vec{ξ}ᵢ^{(j)} is the sparsest vector of coefficients that solves the equation, found using the sequentially thresholding least squares algorithm, which performs linear regressions sequentially, removing the coefficients with values smaller than a given pre-established threshold β.

In conventional methods, a problem arises of having to iterate over all elements of the library of functions and apply the STLSq to all of them. However, depending on the size of the library of functions, the number of different columns can be so large as to increase the computation time to prohibitive limits. In order to overcome this problem, embodiments may operate to associate to each column a probability pⱼ that the corresponding term belongs to the true model. This probability can be initialized with an arbitrary prior, and updated as that given term is selected on successive iterations over different θⱼ. With this information, instead of navigating linearly through the columns of Θ, one can then go through them in an order of decreasing probability. By implementing a stopping criterion, one can early stop the execution once a convergence metric is satisfied.

In various embodiments, the output of a dynamic system learner may be or may include a set of coefficients given a library of functions. It is possible to generalize these coefficients and build an object that represents a dynamical system model, for example, via a dynamical system model process or algorithm. For example, in some embodiments, given multiple input data due to different realization/observations of the system, where N_{P} stands for the number of different realizations of the input. A dynamical system learner may output different models, given different inputs. Assuming Nₘ is the number of different outputted models, the outputted models may be added to a collection of models, which in some embodiments, may be a structure in the code designed to organize the models. Mathematically, the collection of models may be or may include a set of models, where, with μᵢ representing one of the Nₘ different models added to the collection, then [symbol] ={μ₁, μ₂, . . . , μ_{N}_{ₘ}}.

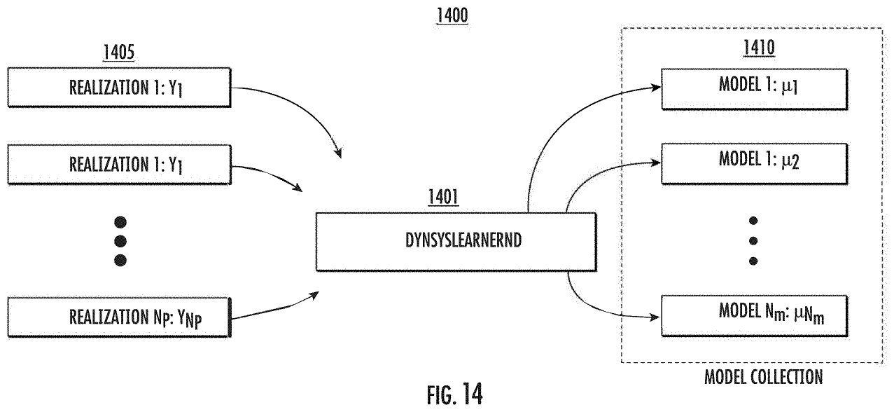

FIG. 14 illustrates an example of a dynamical system model process in accordance with the present disclosure. In various embodiments, process 1400 may correspond with model collections step 804 of FIG. 8.

In some embodiments, process 1400 may include accessing or building a dynamical system learner 1401. In various embodiments, dynamical system learner 1401 may operate to generate a general dynamical system model, given a library of functions, and which can then be fitted to any new data. Dynamical system learner 1401 may receive a set of different realizations 1405, for example, of time series data. A collection of models 1410 may be built from the set of different realizations 1405. In various embodiments, dynamical system learner 1401 may operate to estimate each model parameter for any new data set presented to the model. From model collection 1410, the probability that a given term from the input library of functions is present in the collection may be estimated. For instance, given multiple different models being added to the collection, obtained from different realizations of the system, process 1400 may estimate the probability of a particular term being in any model included in the collection. In various embodiments, process 1400 may also operate to infer a causal diagram from the causal relationships between the variables extracted from the models in collection 1410. In exemplary embodiments, model collection 1410 may be used as a database of models, for example, for complex systems.

Various model or model discovery processes may be used according to various embodiments. A non-limiting example may include a Michaelis-Menten system. In various embodiments, a Michaelis-Menten system may be represented as follows:

$$\dot{x} = {j}_{x} - \frac{{V}_{max} x}{{k}_{m} + x} ,$$

where jₓ=0.6, Vₘₐₓ=1.0, and kₘ=0.3.

In its rational form, the Michaelis-Menten equation can be rewritten as:

$$\dot{x} = \frac{0.18 - 0.4 x}{0.30 + x} ,$$

which, given a library of functions made of second order polynomials for both the numerator and denominator terms, the data-frame representation outputted by a dynamical system learner may be:

| | xdot0 |
|---|---|
| 1 xdot | 0.30 |
| x xdot | 1.00 |
| x\hat{ }2 xdot | 0.00 |
| 1 | −0.18 |
| x | 0.40 |
| x\hat{ }2 | −0.00 |

In some embodiments, for example, in a code or instructions form, a dynamical system model object may be a tuple μ=(Λ, \vec{β}), where Λ is a library of functions, and \vec{β} is a vector of Boolean values representing which terms in the provided library are present in the current model μ. Using the Michaelis-Menten system as an example, its dynamical system model representation may be:

$${μ}_{Michaelis - Menten} = ( [ \begin{matrix}1 \dot{x} \\ x \dot{x} \\ {x}^{2} \dot{x} \\ 1 \\ x \\ {x}^{2}\end{matrix} ] , [ \begin{matrix}True \\ T r u e \\ False \\ T r u e \\ T r u e \\ False\end{matrix} ] ) .$$

Where the terms of the library of functions A that multiply \dot{x} (the first three rows in the example above) correspond to the denominator terms in the actual model represented mathematically in the previous paragraph.

In various embodiments, when instantiating a new dynamical system model, arguments may be passed, for instance, as a data-frame with actual coefficient values. In some embodiments, any coefficient with an absolute value smaller than the machine epsilon (e.g., ˜10⁻¹⁶) may be considered zero; the input data-frame may be simplified. For example, the input data-frame may be simplified using a (Python) sympy.cancel function, which may operate to cancel common factors in a rational function ƒ; and/or for M-dimensional systems, there are M different vectors βᵢ, each one representing the selected models for each variable i. Because the dynamical system model does not store any information regarding the value of the coefficients, two models may be considered equal as long as they have the same terms from the library of functions, i.e., if their \vec{β} and their function library Λ are the same.

Parameter estimation may be performed according to various embodiments. For example, given a data set composed of simultaneous observations of the variable \vec{y} and its derivatives \vec{\dot{y}}, the vector of coefficients ξ=ξ(\vec{y}, \vec{\dot{y}}; Λ) may be obtained given the model μ(Λ, \vec{β}). The regression operator [symbol] is such that:

ξ(\vec{y},\vec{\dot{y}};Λ)= [symbol] (μ(Λ,\vec{β})|\vec{y},\vec{\dot{y}}).

The actual regression, for instance, a coefficient estimation, may be performed through the following steps:
1. The library of functions is fitted to the data, such that a matrix is built where each column corresponds to the application of the respective function from the library applied to the data set. From the Michaelis-Menten example, where the library of functions used was Λ=(1 \dot{y}, x \dot{y}, y² y, 1, y, y²), the corresponding matrix is:

$$Λ ( \vec{y} , \dot{\vec{y}} ) = ( \begin{matrix}\vert & \vert & \vert & \vert & \vert & \vert \\ 1 \dot{y} & y \dot{y} & {y}^{2} \dot{y} & 1 & y & {y}^{2} \\ \vert & \vert & \vert & \vert & \vert & \vert\end{matrix} ) ;$$

2. The appropriate terms from the library of functions are selected according to the binary vector \vec{β}. In the Michaelis-Menten case, this means that only the following columns were selected:

$${Λ}_{β} ( \vec{y} , \dot{\vec{y}} ) = ( \begin{matrix}\vert & \vert & \vert & \vert \\ 1 \dot{y} & y \dot{y} & 1 & y \\ \vert & \vert & \vert & \vert\end{matrix} ) ;$$

3. The coefficients \vec{ξ} should be such that:

Λ_{β}(\vec{x} [symbol] )\vec{ξ}=0,

which can be rewritten as:

[Λ_{β}(y,\dot{y})]₁=[Λ_{β}(y,\dot{y})]_{2, . . . ,N}ξₚₐᵣₜᵢₐₗ,

where N is the number of True elements in the vector \vec{β}, the notation [⋅]ᵢ corresponds to the i-th column of the matrix in between brackets; and the full coefficients from the selected model are given by: \vec{ξ}=\vec{(1,)} [symbol] (−\vec{ξ}ₚₐᵣₜᵢₐₗ), where \vec{(1,)} is the one-dimensional unity vector, and [symbol] is the concatenation operator;
4. Separate the first column from the rest of the matrix:

$$y = {[ {Λ}_{β} ( y , \dot{y} ) ]}_{1} = ( \begin{matrix}\vert \\ 1 \dot{y} \\ \vert\end{matrix} ) X = {[ {Λ}_{β} ( y , \dot{y} ) ]}_{2 , … , N} = ( \begin{matrix}\vert & \vert & \vert \\ y \dot{y} & 1 & x \\ \vert & \vert & \vert\end{matrix} ) ;$$

5. Perform a linear regression: \vec{ξ}ₚₐᵣₜᵢₐₗ= [symbol] (y, X), where \vec{ξ}ₚₐᵣₜᵢₐₗ are the coefficients from the linear regression, without the intercept term.

Parameter estimation for non-linear dynamical systems is a complicated problem to which different approaches have been attempted. For instance, as an optimization problem, in which the parameters (and sometimes initial condition for the variables) are optimized to minimize some objective function, an error measure of some kind, such as an information criterion or likelihood. However, due to its usual high dimensionality, non-linearity and data quality, this problem is usually ill-posed and does not have a simple method that solves it. For that reason, different methods can be used to estimate the parameters in the vector of coefficients ξ. For example, more generally, the problem of parameter estimation can be stated as:

$$ξ = {min}_{ξ} ℰ ( Y , \dot{Y} , f , ξ ) ,$$

where ε is the objective function. It could be the Mean Square Error, or Bayesian Information Criterion, among others.

In various embodiments, the collection of models may not keep repeated models, i.e., if two distinct sets of input data generate the same model (have the same vector \vec{β}), the respective dynamical system model will be added only once to the model collection. However, the information that this particular model was identified twice is not lost by not adding the model two times to the collection. Instead, this information may be stored on the vector of probabilities \vec{π}. The vector \vec{π} has the same dimension as \vec{β}, and its i-th element πᵢ is the probability that the corresponding i-th term from the library of functions was present in one model selected at the many realizations from the input data used to generate the model collection.

In some embodiments, the calculation of the probability vector \vec{π} may be carried out as follows:
1. The model collection is initialized, internally it keeps a vector \vec{κ} that keeps a counter for each term of the library of functions; and a variable n that counts how many models were added to the collection. Both are initialized with zero;
2. When a new model μᵢ is added to the collection:
   a) Increase the value of n by one.
   b) Increase the value of κᵢ (the i-th component of \vec{κ}) by one, if the corresponding term in μᵢ is different from zero.
   c) If the model had already been added to the collection, do nothing; otherwise, add the model to the collection and increase the value of Nₘ by one, where Nₘ is the number of distinct models in the collection;
3. At the end, the probability vector is approximated as \vec{π}=\vec{κ}/n.

In one example, using a Michaelis-Menten process, if the following models were added to the collection (only representing the binary vector \vec{β} of each model): μ₁=(1,0,0,1,1,1)^{T}; μ₂=(1,1,0,1,1,0)^{T}; and μ₃=(1,1,0,1,1,0)^{T}. Then the probabilistic model in this case would be a vector given by: \vec{π}=(1,⅔,0,1,1,⅓)^{T}.

For each model in the collection [symbol] , it is possible to determine a causal diagram (for instance, a confidence matrix). Given a model for a M-dimensional problem μ={Λ,βᵢ}, where i=1, . . . , M, yⱼ causes yᵢ if any non-zero element of βᵢ is associated to a term of the library of functions Λ that contains yⱼ. For example, it is possible to write an equation in which \dot{y}ᵢ=ƒ(yⱼ). If that is true, then the corresponding term of the confidence matrix [symbol] ⱼᵢ is 1, otherwise it is zero.

The causal diagram for the collection of models may be extracted simply by averaging the confidence matrices from all the models that belong to the collection. Consequently, the collection confidence matrix is no longer a binary matrix but a matrix of probabilities, in which the value of the ij element corresponds to the probability that the causal link from yᵢ to yⱼ exists in the collection of models.

In some embodiments, a CKD/ESRD condition analysis process, such as a vascular calcification analysis processes (for instance, process 800 of FIG. 8), may include a model ranking step or process (such as model ranking 805 of FIG. 8). In various embodiments, a model ranking process may operate, given a model collection [symbol] and an input data set {Yᵢ}, to rank, score, or otherwise provide an indication of one or more models based on the data. In one non-limiting example, models with a smaller score (or larger, depending on the scoring system) may be better at describing the dynamics observed in the input data set. In this way, it is possible to rank the models in [symbol] according to their score value.

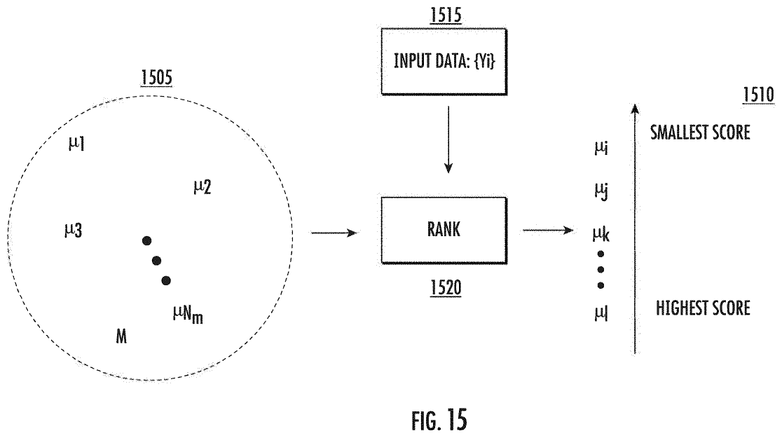

FIG. 15 illustrates an exemplary model ranking process according to the present disclosure. A shown in FIG. 15, a model ranking process may include ranking 1520 a collection of models 1505 according to various ranking criteria to determine a score for one or more of the models in collection 1505. The scored models may be ranked 1510 according to the score. In some embodiments, the ranking criteria may be or may include the ability to fit new input data 1515.

In various embodiments, the models in a model collection may be ranked according to the goodness of fit to any new input data. This model ranking may allow for determining best models for subpopulations of the input data set. For example, given the model collection, the models could be ranked separately for patients with and without diabetes. Use the model collection to build a causal diagram based on the occurrence of specific terms in the best ranked model.

The model score may be determined using various processes. In one non-limiting example, a model score may be determined using a process or based on a process described in Pfister et al., “Learning stable and predictive structures in kinetic systems,” Proceedings of the National Academy of Sciences, 116 (51), pp. 25405-25411(2019) (“Pfister process”), which is incorporated by reference as if fully set forth in the present disclosure. For example, let RSS(ŷ, \vec{y}) be the residual sum of squares between the approximate function ŷ and the noisy data set \vec{y}; and let \vec{\dot{y}}_{μ}=μ(\vec{y}), be the value of the derivative calculated by the model μ with parameters defined from the data. Then the model score TM is given by:

$$TM ( μ ; \vec{y} , \dot{\vec{y}} ) := \frac{RSS ( S [ \vec{y} \vert {\dot{\vec{y}}}_{μ} ] , \vec{y} ) - RSS ( S [ \vec{y} ] , \vec{y} )}{RSS ( S [ \vec{y} ] , \vec{y} )}$$

where S[⋅] corresponds to an smoothing spline approximation, and S[⋅|⋅] corresponds to a derivative constrained smoothing spline.

The model scores may be implemented according to various embodiments. For example, non-limiting implementations may include one or more of: the current score does not measure how well the current model fits to the data, but how well the current model fits the smoothing spline approximation of the data; the constrained smoothing spline is a numerically expensive calculation, since it requires a constrained optimization for the regularization parameter λ, which is significantly slower than a unconstrained optimization required for a regular smoothing spline; the score requires knowledge both of the data and of its derivative, the derivative is necessary to do the regression of the models coefficients; or the current score does not favor sparse models over non-sparse ones, which could lead to overfitting.

In addition, various alternative scores may be used according to exemplary embodiments. For instance, it is possible to use classical model selection criteria, as Bayesian Information Criterion (BIC) or Akaike information criterion (AICc) as long, for example, there is available a good approximation for the data derivative. For example, let Γ(y, y_{predicted}, k) be any arbitrary model selection criterion, where k is the number of parameters selected in the model [symbol] . Then the proposed new score may be determined as follows:

σ(μ;\vec{x},\vec{\dot{x}}):=Γ(S′[\vec{x}],\vec{\dot{x}}_{μ},∥\vec{β}∥₀),

where S′[\vec{x}] corresponds to the first derivative of the spline approximation of the noisy data \vec{x}; [symbol] is the derivative calculated by the model; and ∥\vec{β}∥₀ is the [symbol] ₀ norm of the binary vector \vec{β}, which is simply the number of selected elements from [symbol] .

Using these standard criteria for model selection is advantageous for not relying on the calculation of a constrained smoothing spline. Also, they favor sparse models, and consequently reduces the chance of overfitting. To improve the ability of the method to identify the correct model, it is possible to limit the value of the sparsity degree (SD) such that only models with its SD value smaller than a pre-defined threshold will be included in the list of models to rank. This threshold must be imposed by the user and has the only objective of excluding from the ranking any model with large sparsity degrees, which tend to overfit to the training data and are less representative of the system dynamics and less generalizable.

Dynamical system models and associated methods may be used in various applications according to some embodiments. Non-limiting example applications may include: determining a model that is representative of the system dynamics and can be interpreted physiologically, providing insight to the researcher as to the possible mechanisms underlying the observed interactions between the system observables; building a model that allows forecasts of future values of the system observables, which can then be used to virtually test the effect of different interventions; incorporating the algorithm in a model predictive control loop, in which case the model is determined from data, specifically for each patient, allowing a more precise prediction and accurate intervention; and/or extracting causal information from time series data.

FIG. 16 illustrates an exemplary CKD/ESRD condition analysis processes according to a second embodiment in accordance with the present disclosure. As shown in FIG. 16, a CKD/ESRD condition analysis process may be or may include a dynamic system modeler process 1600 configured for model selection and causal analysis.

Dynamical system modeling algorithms may use a library of test functions onto which they perform a sparsity promoting regression with the data. In some embodiments, a function library 1605 may be an input of process 1600. In various embodiments, function library 1605 may be or may include a collection of N_{F} functions ƒᵢ: [symbol] , represented as Λ=(ƒ₁, ƒ₂, . . . , θ_{N}_{_{F}}). As an example, if function library 1605 corresponds to all polynomial combinations up to the second-order of two state vectors (x₁ and x₂), then Λ=(1, x₁, x₂, x₁², x₂², x₁x₂).

A function library Λ may be an operator on the data, given a matrix of data: X=(x₁ x₂ . . . x_{N}) where each row represents an observation of the state variables at a given time. Then the operator Λ=(ƒ₁, ƒ₂, . . . , ƒ_{N}_{_{F}}) applied on X and \dot{x}_{ι}, denoted as Λ(\dot{x}_{ι}, X)=(ƒ₁(X)\dot{x}_{ι} ƒ₂(X)\dot{x}_{ι} . . . ƒ₁(X) ƒ₂(X)), is a matrix where each column corresponds to the application of a different function library 1605 on the data and each row corresponds to a different time.

Patient data 1606 for process 1600 may be heterogeneous and noisy, for example, such that some variables have missing values (represented by NA) and the values that are missing are not in the same times for all variables. The data matrix Y may have each row of the matrix referring to a different time, and each column referring to a different variable:

$$Y = ( \begin{matrix}{y}_{1} ( {t}_{1} ) & {y}_{2} ( {t}_{1} ) & {y}_{3} ( {t}_{1} ) & … & {y}_{N} ( {t}_{1} ) \\ NA & {y}_{2} ( {t}_{2} ) & NA & … & {y}_{N} ( {t}_{2} ) \\ NA & {y}_{2} ( {t}_{3} ) & {y}_{3} ( {t}_{3} ) & … & {y}_{N} ( {t}_{3} ) \\ ⋮ & ⋮ & ⋮ & ⋱ & ⋮ \\ {y}_{1} ( {t}_{L} ) & {y}_{2} ( {t}_{L} ) & {y}_{3} ( {t}_{L} ) & … & {y}_{N} ( {t}_{L} )\end{matrix} ) .$$

Model selection 1610 may receive as input function library 1605 (e.g., the library of functions Λ) and patient data 1606 from all the patients in each population (each patient data denoted Y) and builds a model collection 1607 (e.g., collection of models [symbol] =μ₁, μ₂, . . . ). Each model may include or may be a Boolean matrix where each column refers to different state variables and each row informs whether the corresponding element from function library 1605 was selected.

In one non-limiting example, function library 1605, with second-order polynomial for two variables, may be selected:

$${\dot{x}}_{1} = {ξ}_{11} {x}_{1} + {ξ}_{12} {x}_{1} {x}_{2} \\ {\dot{x}}_{2} = \frac{{ξ}_{21} {x}_{2} + {ξ}_{22} {x}_{1}^{2}}{{ξ}_{23} + {ξ}_{24} {x}_{1}} ,$$

where ξᵢⱼ are coefficients. Then, the model to be incorporated into model collection [symbol] would be:

| Library Λ | x₁ | x₂ |
|---|---|---|
| \dot{x}1 | T | T |
| \dot{x}x₁ | F | T |
| \dot{x}x₂ | F | F |
| \dot{x}x₁² | F | F |
| \dot{x}x₂² | F | F |
| \dot{x}x₁x₂ | F | F |
| 1 | F | F |
| x₁ | T | F |
| x₂ | F | T |
| xx₁² | F | T |
| xx₂² | F | F |
| xx₁x₂ | T | F. |

For any given data matrix Y with N state variables, and its derivatives given by \dot{Y} and a given function library Λ, the output model μ is given by:

μ=Γ(Y,\dot{Y};Λ),

where Γ is the model selection operator.

The operator Γ requires its input data Y and its derivatives \dot{Y} to be complete, i.e., there cannot be missing values in any of the times passed to the operator. In order to overcome this difficulty, an iterative use of smoothing splines may be used, defined as: Smoothing splines: for each noisy variable yᵢ(t) with i=1, . . . , N, the smoothing spline is given by:

$$S [ {y}_{i} ] = {arg min}_{y ∈ {ℋ}_{C}} {\sum }_{ℓ = 0}^{L} {( {y}_{i} ( {t}_{ℓ} ) - y ( {t}_{ℓ} ) )}^{2} + λ \int {\ddot{y} ( τ )}^{2} d τ ,$$

where λ is a regularization parameter, [symbol] _{C} is the space of all smooth functions for which values and the first two derivatives are bounded in absolute value by C (for instance, according to the Pfister process). For Smoothing splines with derivative constraints: a similar procedure can be done, however with a constraint imposed in the derivatives \dot{y}_{ι}:

S[yᵢ|\dot{y}_{ι}]=S[yᵢ] s.t. \dot{y}( [symbol] )=\dot{y}_{ι}( [symbol] ).

With these two operators, the model selection algorithm may operate as follows (the indexes represent the algorithm iteration):
1. Calculate spline on noisy data: X₀=S[Y];
2. Calculate the first model estimate μ⁰=Γ(X⁰, \dot{X}⁰; Λ);
3. Calculate derivative at data points with the obtained model: \dot{Y}ᵢ=μᵢ₋₁(Y);
4. Calculate constrained smoothing spline Xᵢ=S[Y|\dot{Y}ᵢ];
5. Update the model estimate μᵢ=Γ(Xᵢ, \dot{X}ᵢ; Λ);
6. Repeat steps 3 to 5 until a convergence criterion is satisfied;
7. Add μᵢ to the model collection [symbol] .

A model selection operator Γ may follow a procedure in which, given the data X, its derivative \dot{X}, and the appropriate function library Λ, to find the best model is to solve the following constrained optimization problem for each column \dot{x}ᵢ of \dot{X}:

$${min}_{Ξ} {‖ Λ ( X , {\dot{x}}_{i} ) - Λ ( X , {\dot{x}}_{i} ) Ξ ‖}_{2} + β {‖ Ξ ‖}_{0} s . t . diag Ξ = 0 ,$$

where the selected model coefficients correspond to the columns of Ξ with a sparse representation that allows accurate prediction of \dot{x}ᵢ given \dot{X}.

Solving this minimization problem is very difficult due to its non-convexity. However, it can be rewritten as a set of 2×N_{F} convex problems:

λⱼ(X,\dot{x}ᵢ)=Λ(X,\dot{x}ᵢ|λⱼ)ξⱼ,

where λⱼ is the j-th column of Λ; and Λ(⋅|λⱼ) is the library of functions without its j-th column. Then, we solve this problem by applying the sequential threshold least square algorithm to find a sparse solution ξⱼ.

The choice of which set of coefficients ξⱼ is the actual solution to the system may be made by one of many different model selection criteria, including, without limitation, AIC, BIC, or derivates thereof. In principle, this optimization problem must be solved 2×N_{F} times, one for each element of the function library. However, it is possible to build a probability that a given element j of the function library participates in the system dynamics. This probability is updated at every new ξ calculated, allowing the determination of some early stop criteria.

For performance gains, some embodiments may operate to reduce the number of times model selection 1610 is called for a given population. It does not need to be calculated for all the patients in the population and it also does not need to run every time the algorithm is executed. Model selection 1610 may be run as many times as necessary to build a comprehensive model collection 1607 for the given problem. With model collection 1607 (or [symbol] ), model ranking 1615 may operate to rank the models according to their prediction power in a particular population of patients.

For a given model μᵢ in model collection 1607, the actual coefficient values of the model for each patient (or patient(s) of interest) of the population may be calculated. For example, with Λ_{μ}_{ᵢ} the library of functions Λ reduced by μᵢ, i.e., selecting only the columns from Λ that are present in μᵢ, then Λ_{μ}_{ᵢ} is the matrix built with only these selected columns. For example, the reduced library for x₁ would be Λ_{μ}(X, \dot{x}₁)=(\dot{x} x₁ x₁x₂).

In some embodiments, the process or algorithm to find the model coefficients may include:
1. Calculating spline on noisy data X₀=S[Y];
2. Calculating the first estimate of model coefficients ξ,

$$by : {min}_{ξ} {‖ {Λ}_{μ} ( {X}_{0} , {\dot{X}}_{0} ) ξ ‖}_{2} ;$$

3. Calculating derivative at data points with the model: \dot{Y}ᵢ=ƒ_{μ}(Y, ξᵢ₋₁);
4. Calculating constrained smoothing spline Xᵢ=S[Y|\dot{Y}ᵢ];
5. Calculating a new estimate of the coefficients:

$${min}_{ξ} {‖ {Λ}_{μ} ( {X}_{1} , {\dot{X}}_{1} ) ξ ‖}_{2} ;$$

6. Repeat steps 3 through 5 until a convergence criterion is satisfied.

Each model in model collection 1607 may be scored according to the following equation, where the sum is through all the Nₚ patients in the population:

$$T ( μ ) = \frac{1}{N} {\sum }_{i = 1}^{{N}_{p}} \frac{RSS ( S [ Y \vert {f}_{μ} ( Y , {ξ}_{i} ) ] ) - RSS ( {X}_{0} )}{RSS ( {X}_{0} )} ,$$

where RSS is the residual sum of squares, ƒ_{μ}(Y, ξᵢ) is the derivative calculated by the model μ with the coefficients ξᵢ calculated for patient i, and X₀ is the unconstrained smoothing spline of the data. In some embodiments, the model with the lowest score is the model that best represents the population, from all the models in the model collection, as it shows a more stable fit across different patients.

The model selection process or algorithm may operate to score the variables as they might be present in some models but not in others. A process the same or similar to the Pfister process may be used to rank the variables according to their relevance in the best scoring models. For instance, given the K top-ranked models, the following score may be assigned to the j-th variable:

sⱼ=Fraction of the top K models that depend on xⱼ.

In various embodiments, the relevance of a given variable may be interpreted as a robust measure of causality, i.e., if a model for xᵢ depends on xⱼ, then we can say that xⱼ causes xᵢ, in a sense that changes in xⱼ lead to changes in xᵢ. The score sⱼ measures the fraction of the top-scoring models that captured this causal relation.

### Experiment I—Dynamical System Learner Benchmark Results

A series of tests made with data generated by known models was performed for the dynamical system learner to show the performance of the dynamical system learner under different conditions. In order to assess the quality of the system output, the following series of benchmark metrics were calculated under different conditions of the data:
True Positive Rate (TPR)—The proportion of True terms that were correctly selected, larger is better;
False discovery rate (FDR)—The proportion of selected terms that were not correctly selected, smaller is better;
Sparsity degree (SD)—Proportion of selected terms, smaller (but not zero) is better;
Causal Stability (CS)—Proportion of different terms in the confidence matrix, smaller is better.
RSS Residual sum of squares, normalized to the size of the array. The RSS is calculated with the original data X,
RSS noise (nRSS) RSS calculated with the noise data with missing values. The missing values are linearly interpolated when calculating the value of the derivative with the model.

The data used in this benchmark was generated according to the following steps: (1) Generate data X and \dot{X} from nᵢ initial conditions; (2) Add Gaussian Noise: \tilde{X}=X+ [symbol] (μ=0, σ=ρΔX), where ΔX is the amplitude of the input time series, and ρ is a factor representing the value of the added noise standard deviation in terms of the percentage of the time series amplitude. [symbol] (μ, σ) is a Gaussian noise with mean μ and variance σ²; (3) Define a library of functions: Λ; (4) Calculate the model μ with dynamical system learner (or DynSysLearner) μ=Γ(X, \dot{X}, Λ; β), where β is the regularization_parameter, and Γ is the model selection operator, in other words, it is the mathematical representation of the dynamical system learner method; (5) Calculate benchmark metrics as a function of the regularization parameter [symbol] = [symbol] (β).

Every model discovered by the DynSysLearner is saved to a collection of models. These saved models can then be ranked according to the value of a model score (e.g., via the Pfister process), and described by the following equation:

$$T ( M ) = \frac{1}{n} {\sum }_{i = 1}^{n} \frac{RSS ( {\hat{y}}_{constrained}^{( i )} ) - RSS ( {\hat{y}}^{( i )} )}{RSS ( {\hat{y}}^{( i )} )} ,$$

where n corresponds to the number of different observations of a given system, i.e., different time series that represent the same system (e.g., data from different patients, or different observations of the same experimental conditions for a given experiment). RSS is the residual sum of squares ŷ is a smoothing spline approximation of the time series calculated from the noisy data, and ŷ_{constrained} is the smoothing spline approximation of the data constrained to the values of derivatives calculated by the discovered model in the model collection.

This score is then calculated for all the models in the model collection, and the model with the smallest score is considered the one with the most stable fit. Considering that each model is saved in the model collection only with the information about which terms of the library of functions are present in the model, a linear regression may be calculated on the data to estimate the values of the coefficients, for each different input data set.

A set of benchmarks were made using a different score to rank the models: the Bayesian Information Criterion: BIC( [symbol] ; x, \dot{x})=BIC(S′^{[x]}, [symbol] , k), where S′[x] is the derivative of the smoothing spline approximation to the data, \dot{x}_{μ} is the derivative calculated from the model; and k is the number of non zero coefficients from the selected model. The appropriate value of the regularization parameter for the smoothing spline is calculated using a k-fold cross-validation.

In this Experiment I, benchmark results were determined for four different models: (i) The Michaelis-Menten model; (ii) the Selkov system; (iii) the Lorenz system; and (iv) a model describing Osteoblast-Osteoclast activity (or bone remodeling system).

The equation defining the Michaelis-Menten model is:

$$\dot{x} = {j}_{x} - \frac{{V}_{{max}^{x}}}{{k}_{m} + x} ,$$

where jₓ=0.6, Vₘₐₓ=1.0, kₘ=0.3. An appropriate integration time may be Δt=5 a. u. t., where a.u.t. stands for arbitrary units of time. The determination of the integration time was made as to ensure that the time series passed to the algorithm has not yet reached a steady state.

In order to determine the most appropriate value of the regularization parameter, the system was benchmarked without noise and without any pre-processing for different values of the STLSQ threshold β, and without optimizing the value of β for each run. The following simulation parameters were used:

| Parameter | Value |
|---|---|
| Number of points for each time series | 100 |
| Time of integration for each time series | 5 |
| Number of different initial conditions | 25 |
| Percentage of amplitude of added noise (ρ) | 0 |

As expected, if β is too small the method fails to determine the correct model. However, as β increases, all the appropriate metrics increase accordingly suggesting that the DynSysLearner successfully found the correct equation. However, if the value of β is increased beyond a given threshold, then real terms are not selected, which deteriorates the quality of the benchmark metrics. The chosen value for β was 0.75.

For systems that fall too rapidly to a steady state (fixed point), only the short lived transient dynamics is relevant for model discovery. In the case that the underlying system is too complex, it may be necessary to simulate more than one random initial condition to increase the information on the system dynamics. Even for a system as complex as the Michaelis-Menten model, only one initial condition (with 100 data points) may be enough to obtain the correct model via DynSysLearner.

Benchmarks were determined for the model discovery method when the input time series is more realistic and has noise. In order to mimic such behavior, Gaussian noise was added to the time series and its derivative:

X′=X+ [symbol] (0,ρamp(X))

\dot{X}′=\dot{X}+ [symbol] (0,ρamp(\dot{X}))′

where [symbol] (μ, σ) is a Gaussian distribution with mean μ and standard deviation σ, and amp(⋅) is the amplitude operator. In this way ρ represents the ratio between the added noise standard deviation and the time series amplitude, and it is referred to as the percentage_of_amplitude. In the benchmarks, only noise levels as large as 10⁻³ start to disrupt the quality of the benchmark, suggesting that the method is robust against noise.

Regarding the presence of noise for models with rational form and the value of the regularization parameter, because both numerator and denominator can be re-scaled by a common factor, the value of the coefficients are not fixed. That means that the value of the regularization parameter may greatly affect the performance of the method.

The determination of the regularization parameter may be useful for the success of the dynamical system learner process, in which case it would be desirable to have a non-arbitrary way of determining it. For that reason, the value of the regularization parameter can be optimized to minimize one of three implemented metrics for model selection: Cross Validation; Bayesian Information criterion (BIC); or Akaike Information criterion (AIC)

For no-preprocessing benchmarks, a total of 2 distinct models were selected from all the realizations with different parameters, and the true model is in the collection of models. When ranked over a data set with 5 realizations of the system (5 initial conditions), with each realization with 100 points, without added noise, the true model is the model with the lowest rank in the collection, with rank value equal to 1.78518147×10⁰¹.

Using a collection of models with 7 different models (generated from noisy data set), one of them being the true model, using the following parameters when generating the noisy data to rank the models:

| Parameter | Value |
|---|---|
| Number of points per time series | 100 |
| Time of integration | 5 |
| Number of different initial conditions | 10 |
| Percentage of amplitude of added noise (ρ) | 0.01 |
| Rate of missing values in the input data | 0.25 |

Under these conditions and with 25% of missing values, using Bayesian Information Criterion to rank the models, the method is capable of correctly identifying the true model.

The equation defining the Selkov model is:

\dot{x}=−x+ay+x²y

\dot{y}=b−ay−x²y,

where: a=0.1 and b=0.41. An appropriate integration time may be Δt=25 a. u. t., where a.u.t. stands for arbitrary units of time.

Benchmark simulations were determined where no noise is added to the time series. In order to determine the most appropriate value of the regularization parameter, the system was benchmarked without noise and without any pre-processing for different values of the STLSQ threshold β. The following simulation parameters were used:

| Parameter | Value |
|---|---|
| Number of points for each time series | 100 |
| Time of integration for each time series | 25 |
| Number of different initial conditions | 25 |
| Percentage of amplitude of added noise (ρ) | 0 |

The results are very robust or small values of the regularization parameter, only when β increases to values with magnitude comparable to that of the real coefficients of the system (β˜0.4) is that the method begins to fail. The value of β chosen for the subsequent benchmarks was β=0.05. In addition, the values of the benchmark metrics are insensitive to the number of initial conditions.

Gaussian noise was added to the time series for the Selkov model using the same prescription already explained in the respective section for the Michaelis-Menten model. The noiseless case are robust to noise levels as large as ˜10⁻⁴ of the input time series amplitude.

The optimization conditions used for the Selkov system are similar to the ones used for the Michaelis-Menten system, the difference here is that having two equations, when the optimize parameter from the fit method from the DynSysLearnerND object is set to ‘all’, it means that all the regularization parameters (\vec{β}=(β₀, β₁)^{T}) are going to be optimized separately, consequently the value of the regularization parameter for each equation might be different.

For results with BIC, a total of 21 distinct models were identified and added to the collection of models. The true model belongs to the final collection of models. The following parameters were used when generating the data to rank the model:

| Parameter | Value |
|---|---|
| Number of points per time series | 100 |
| Time of integration | 25 |
| Number of different initial conditions | 5 |
| Percentage of amplitude of added noise (ρ) | 0.01 |
| Rate of missing values in the input data | 0.25 |

In this condition, the BIC was able to correctly rank the true model. The true model was also correctly ranked when the noise standard deviation was 10% of the time series amplitude and there was 50% missing values, in which case a total of 10 different initial conditions were used.

The Lorenz system is a system used in the study of non-linear dynamics. The equations defining it are:

\dot{x}=σ(y−x)

\dot{y}=x(ρ−z)−y,

ż=xy−βz

where the values of the coefficients are given by: ρ=28, σ=10, β=8/3.

The Lorenz system is very stable when subjected to the DynSysLearner, as the true positive rate is 1 and the false discovery rate is zero for a very large range of values of the regularization parameter. As the performance of the method seems to be indifferent to the value of β, within the interval of values calculated in this study. The value β=0.05 was kept fixed in all the subsequent benchmarks. Regarding the number of initial conditions, the results for the Lorenz system is consistent to the results observed previously for the other systems: the performance of the DynSysLearner seems to be indifferent to the number of initial conditions used, as long as the number of points sampled to the algorithm is large enough.

The algorithm is robust against the addition of noise for noise standard deviations as large as 10⁻³ of the input time series amplitude. By increasing the amplitude of the added noise, first the number of falsely discovered terms increases and it is only at ρ˜10⁻² that the true positive rate starts to decrease.

When optimizing the regularization parameter there are two different strategies implemented: (i) if the value of the optimize parameter is set to ‘all’, then the regularization parameter is going to be optimized separately for each equation in the model; (ii) if the value of optimize is set to ‘first’, then the regularization parameter of the first equation is optimized and its value is used for the subsequent equations. Collection of models

There were 27 different models saved in the model collection for the Lorenz system. The true model is present in this model collection. When ranked with a noiseless data set, the model with the lowest score is not the true model. Under these conditions the model with the lowest rank corresponds to the following set of equations:

\dot{x}=α₁+α₂x+α₃y+α₄yz

\dot{y}=β₁+β₂x+β₃y+β₄yz,

ż=γ₁

where α, β and γ are constants. Increasing the amplitude of the noise in the input data to 1% of the input time series amplitude, the model with the lowest rank corresponds to the following set of equations:

\dot{x}=α₁+α₂x+α₃y+α₄xz+α₅yz

\dot{y}=β₁+β₂x+β₃y+β₄xz

ż=γ₁+γ₂z+γ₃xy+γ₄y²

where α, β and γ are constants.

For BIC, the following are the parameters used when ranking the models for the Lorenz system. There were 38 models in the model collection.

| Parameter | Value |
|---|---|
| Number of points per time series | 100 |
| Time of integration | 100 |
| Number of different initial conditions | 5 |
| Percentage of amplitude of added noise (ρ) | 0.0 |
| Rate of missing values in the input data | 0.0 |

In this noiseless case, the true model is correctly identified as the highest ranked model. When PERCENTAGE_OF_AMPLITUDE=0.01, then the highest ranked model is no longer the correct model. However, reducing the time of integration to 10 allows the system to correctly rank the true model. In this case, due to the higher frequencies present in the Lorenz system time series, the smoothing spline step may have difficulties converging to a good approximation of the system, jeopardizing the quality of the scoring of the models. Using the smaller time spam leads to less oscillations in the observed time series, allowing for better convergence of the smoothing spline. In this condition, even setting the rate of missing values in the input data to 25% allows the correct identification of the true model.

Given the following parameters:

| Parameter | Value |
|---|---|
| Number of points per time series | 100 |
| Time of integration | 10 |
| Number of different initial conditions | 50 |
| Percentage of amplitude of added noise (ρ) | 0.1 |
| Rate of missing values in the input data | 0.5 |

the true model was correctly identified with the highest rank. Note that it was necessary to increase the number of initial conditions, in other words, it was necessary to increase the amount of data for the true equation to be correctly ranked, given the high noise amplitude and missing value rate.

A model describing Osteoblast-Osteoclast activity, the same or similar to the model described in Lemaire et al., “Modeling the interactions between osteoblast and osteoclast activities in bone remodeling,” Journal of Theoretical Biology, 229(3), pp. 293-309 (2004), was performed. The following is a description of the model:

$${π}_{c} = \frac{C + {f}_{0} {C}^{s}}{C + {C}^{s}} \\ {D}_{B} = {f}_{0} {d}_{B} \\ \overline{P} = {I}_{P} / {k}_{P} \\ {P}^{0} = {S}_{P} / {k}_{P} \\ {P}^{S} = {k}_{6} / {k}_{5} \\ {π}_{P} = ( \overline{P} + {P}^{0} ) / ( \overline{P} + {P}^{S} ) \\ {π}_{L} = \frac{{k}_{3}}{{k}_{4}} \frac{{K}_{L}^{P} {π}_{P} B}{1 + \frac{{k}_{3} K}{{k}_{4}} + \frac{{k}_{1}}{{k}_{2} {k}_{O}} ( \frac{{K}_{O}^{P} R}{{π}_{P}} + {I}_{O} )} ) ( 1 + \frac{{I}_{L}}{{r}_{L}} )$$

The following are the equations for the model:

$$\dot{R} = {D}_{R} {π}_{C} - \frac{DBR}{{π}_{C}} \\ \dot{B} = \frac{{D}_{B} R}{{π}_{C}} - {k}_{B} B \\ \dot{C} = {D}_{C} {π}_{L} - {D}_{A} {π}_{C} C$$

The values used for the parameters are:

| Parameter | Value |
|---|---|
| I_{L} | 0 |
| I_{O} | 0 |
| I_{P} | 0 |
| C^{s} | 5e−3 |
| D_{A} | 0.7 |
| d_{B} | 0.7 |
| D_{C} | 2.1e−3 |
| D_{R} | 7e−4 |
| f₀ | 0.05 |
| K | 10 |
| k₁ | 1e−2 |
| k₂ | 10 |
| k₃ | 5.8e−4 |
| k₄ | 1.7e−2 |
| k₅ | 0.02 |
| k₆ | 3 |
| k_{B} | 0.189 |
| K_{L}^{P} | 3e6 |
| k_{O} | 0.35 |
| K_{O}^{P} | 2e5 |
| k_{P} | 86 |
| r_{L} | 1e3 |
| S_{P} | 250 |

When not explicitly mentioned, the following parameters were used in the simulations:

| Parameter | Value |
|---|---|
| Number of points per time series | 100 |
| Time of integration | 20 |
| Number of different initial conditions | 25 |
| OPTIMIZE | ‘none’ |
| Percentage of amplitude of added noise (ρ) | 0.0 |
| Regularization parameter (β) | 0.8 |

Only a few additional initial conditions is sufficient to help the algorithm to find the right model.

Given an added noise with standard deviation corresponding to 10⁻⁸ and 10⁻⁴ of the time series amplitude, a term by term comparison of the differences between the calculated and the true model is provided. To make the comparison easier, both the calculated and true model were normalized in their data frame representation, i.e., the following (Python) operation was performed:

```
calculated_eqs = sympyfy_model(
  learner.coefficients / learner.coefficients.max( ))
true_eqs = sympyfy_model(
  true model / true_model.max( )),
```

where the sympyfy_model uses the sympy.cancel to simplify the model in its rational function representation.

The results presented in the following sections are organized in tables in which each row refers to a different term from the library of functions. The first column represents the values of the coefficients for of the true equation; the second column are the coefficients of the terms that are missing in the model selected; and the in the third column are the coefficients of the terms that were selected but that are not present in the true equation of the system.

Equation for R, ρ=10⁻⁸:

| | True | Missing | Extra |
|---|---|---|---|
| 1 xdot | 1.25e06 | 0 | 0 |
| R xdot | 0 | 0 | 0 |
| B xdot | 0 | 0 | 0 |
| C xdot | 0.00525 | 0 | 0 |
| R\hat{ }2 xdot | 0 | 0 | 0 |
| R B xdot | 0 | 0 | 0 |
| R C xdot | 0 | 0 | 0 |
| B\hat{ }2 xdot | 0 | 0 | 0 |
| B C xdot | 0 | 0 | 0 |
| C\hat{ }2 xdot | 1 | 0 | 0 |
| 1 | −4.375e−11 | −4.375e−11 | 0 |
| R | 8.75e07 | 0 | 0 |
| B | 0 | 0 | 0 |
| C | −3.5e07 | 0 | 0 |
| R\hat{ }2 | 0 | 0 | 0 |
| R B | 0 | 0 | 0 |
| R C | 0.00035 | 0 | 0 |
| B\hat{ }2 | 0 | 0 | 0 |
| B C | 0 | 0 | 0 |
| C\hat{ }2 | 0.0007 | 0 | 0 |
| R\hat{ }3 | 0 | 0 | 0 |
| R\hat{ }2 B | 0 | 0 | 0 |
| R\hat{ }2 C | 0 | 0 | 0 |
| R B\hat{ }2 | 0 | 0 | 0 |
| R B C | 0 | 0 | 0 |
| R C\hat{ }2 | 0.035 | 0 | 0 |
| B\hat{ }3 | 0 | 0 | 0 |
| B\hat{ }2 C | 0 | 0 | 0 |
| B C\hat{ }2 | 0 | 0 | 0 |
| C\hat{ }3 | 0 | 0 | 0 |

For this small noise amplitude, the result for \dot{R} is satisfactory, since there are no extra terms and the only missing term has a very small amplitude.

Equation for B, ρ=10⁻⁸:

| | True | Missing | Extra |
|---|---|---|---|
| 1 xdot | 0.00025 | 0 | 0 |
| R xdot | 0 | 0 | 0 |
| B xdot | 0 | 0 | 0 |
| C xdot | 1 | 0 | 0 |
| R\hat{ }2 xdot | 0 | 0 | 0 |
| R B xdot | 0 | 0 | 0 |
| R C xdot | 0 | 0 | 0 |
| B\hat{ }2 xdot | 0 | 0 | 0 |
| B C xdot | 0 | 0 | 0 |
| C\hat{ }2 xdot | 0 | 0 | 37120.3 |
| 1 | 0 | 0 | 0 |
| R | 0.000175 | 0 | 0 |
| B | 4.725e05 | 4.725e05 | 0 |
| C | 0 | 0 | 0 |
| R\hat{ }2 | 0 | 0 | 0 |
| R B | 0 | 0 | 0 |
| R C | 0.035 | 0 | 0 |
| B\hat{ }2 | 0 | 0 | 0 |
| B C | 0.189 | 0 | 0 |
| C\hat{ }2 | 0 | 0 | 0 |
| R\hat{ }3 | 0 | 0 | 0 |
| R\hat{ }2 B | 0 | 0 | 0 |
| R\hat{ }2 C | 0 | 0 | 0 |
| R B\hat{ }2 | 0 | 0 | 0 |
| R B C | 0 | 0 | 0 |
| R C\hat{ }2 | 0 | 0 | −1299.21 |
| B\hat{ }3 | 0 | 0 | 0 |
| B\hat{ }2 C | 0 | 0 | 0 |
| B C\hat{ }2 | 0 | 0 | 7015.74 |
| C\hat{ }3 | 0 | 0 | 0 |

In this case, the extra terms all have the C² term in common, meaning that they could be simplified and lead only to terms proportional to R and B in the function numerator, which are terms present in the original equation.

Equation for C, ρ=10⁻⁸

| | True | Missing | Extra |
|---|---|---|---|
| 1 xdot | 0.00670588 | 0 | 0 |
| R xdot | 147.429 | 0 | 0 |
| B xdot | 0 | 0 | 0.356341 |
| C xdot | 1.34118 | 0 | 0 |
| R\hat{ }2 xdot | 0 | 0 | 0.32171 |
| R B xdot | 0 | 0 | 0.146277 |
| R C xdot | 29485.7 | 0 | 0 |
| B\hat{ }2 xdot | 0 | 0 | 0.148223 |
| B C xdot | 0 | 0 | 1.36504 |
| C\hat{ }2 xdot | 0 | 0 | −3.27192 |
| 1 | 0 | 0 | 0 |
| R | 0 | 0 | 0 |
| B | 0.0208276 | 0 | 0 |
| C | 0.000234706 | 0.000234706 | 0 |
| R\hat{ }2 | 0 | 0 | 0 |
| R B | 0 | 0 | 0 |
| R C | 5.16 | 0 | 0 |
| B\hat{ }2 | 0 | 0 | 0 |
| B C | −4.16553 | 0 | 0 |
| C\hat{ }2 | 0.938824 | 0 | 0 |
| R\hat{ }3 | 0 | 0 | 0 |
| R\hat{ }2 B | 0 | 0 | 0 |
| R\hat{ }2 C | 0 | 0 | 0.222141 |
| R B\hat{ }2 | 0 | 0 | 0 |
| R B C | 0 | 0 | 0.100044 |
| R C\hat{ }2 | 20640 | 0 | 0 |
| B\hat{ }3 | 0 | 0 | 0 |
| B\hat{ }2 C | 0 | 0 | 0.103737 |
| B C\hat{ }2 | 0 | 0 | 0.964054 |
| C\hat{ }3 | 0 | 0 | −2.27439 |

The solution for C presents a large number of extra terms, suggesting that the method had difficulties finding a sparse solution. This is probably explained by the fact that the dynamics for C rapidly converges to a steady state, conveying not enough information on the system dynamics.

Equation for R, ρ=10⁻⁴

| | True | Missing | Extra |
|---|---|---|---|
| 1 xdot | 1.25e06 | 0 | 0 |
| R xdot | 0 | 0 | 0.360919 |
| B xdot | 0 | 0 | −1.84893 |
| C xdot | 0.00525 | 0 | 0 |
| R\hat{ }2 xdot | 0 | 0 | 0.131712 |
| R B xdot | 0 | 0 | 0.539312 |
| R C xdot | 0 | 0 | −711.453 |
| B\hat{ }2 xdot | 0 | 0 | 2.80775 |
| B C xdot | 0 | 0 | −688.009 |
| C\hat{ }2 xdot | 1 | 0 | 0 |
| 1 | −4.375e−11 | −4.375e−11 | 0 |
| R | 8.75e07 | 0 | 0 |
| B | 0 | 0 | 0 |
| C | −3.5e07 | 0 | 0 |
| R\hat{ }2 | 0 | 0 | 0.208772 |
| R B | 0 | 0 | 0.258294 |
| R C | 0.00035 | 0 | 0 |
| B\hat{ }2 | 0 | 0 | 0.021849 |
| B C | 0 | 0 | 0.589835 |
| C\hat{ }2 | 0.0007 | 0 | 0 |
| R\hat{ }3 | 0 | 0 | 0.0451252 |
| R\hat{ }2 B | 0 | 0 | 0.0430918 |
| R\hat{ }2 C | 0 | 0 | −24.9532 |
| R B\hat{ }2 | 0 | 0 | 0.106825 |
| R B C | 0 | 0 | −24.0966 |
| R C\hat{ }2 | 0.035 | 0 | 0 |
| B\hat{ }3 | 0 | 0 | 0 |
| B\hat{ }2 C | 0 | 0 | 0.108237 |
| B C\hat{ }2 | 0 | 0 | 0.0226103 |
| C\hat{ }3 | 0 | 0 | 0.1334 |

The increase in the noise amplitude leads the algorithm to misidentify a significant number of extra terms, producing a non-sparse solution.

Equation for B, ρ=10⁻⁴

| | True | Missing | Extra |
|---|---|---|---|
| 1 xdot | 0.00025 | 0.00025 | 0 |
| R xdot | 0 | 0 | 0 |
| B xdot | 0 | 0 | −20.3642 |
| C xdot | 1 | 0 | 0 |
| R\hat{ }2 xdot | 0 | 0 | 0 |
| R B xdot | 0 | 0 | 7.27951 |
| R C xdot | 0 | 0 | −967.182 |
| B\hat{ }2 xdot | 0 | 0 | 40.6743 |
| B C xdot | 0 | 0 | 2605.89 |
| C\hat{ }2 xdot | 0 | 0 | 8030.61 |
| 1 | 0 | 0 | 0 |
| R | 0.000175 | 0.000175 | 0 |
| B | 4.725e05 | 4.725e05 | 0 |
| C | 0 | 0 | 0 |
| R\hat{ }2 | 0 | 0 | 0 |
| R B | 0 | 0 | 0 |
| R C | 0.035 | 0 | 0 |
| B\hat{ }2 | 0 | 0 | −3.94657 |
| B C | 0.189 | 0 | 0 |
| C\hat{ }2 | 0 | 0 | 0 |
| R\hat{ }3 | 0 | 0 | 0 |
| R\hat{ }2 B | 0 | 0 | 0 |
| R\hat{ }2 C | 0 | 0 | 34.2654 |
| R B\hat{ }2 | 0 | 0 | 0 |
| R B C | 0 | 0 | −273.94 |
| R C\hat{ }2 | 0 | 0 | −280.977 |
| B\hat{ }3 | 0 | 0 | 7.77352 |
| B\hat{ }2 C | 0 | 0 | 492.304 |
| B C\hat{ }2 | 0 | 0 | 1517.33 |
| C\hat{ }3 | 0 | 0 | 0 |

For the \dot{B} equation, the same consideration is valid, the obtained solution has too many extra terms and is non-sparse.

Equation for C, ρ=10⁻⁴

| | True | Missing | Extra |
|---|---|---|---|
| 1 xdot | 0.00670588 | 0.00670588 | 0 |
| R xdot | 147.429 | 147.429 | 0 |
| B xdot | 0 | 0 | 0 |
| C xdot | 1.34118 | 0 | 0 |
| R\hat{ }2 xdot | 0 | 0 | 0 |
| R B xdot | 0 | 0 | 0 |
| R C xdot | 29485.7 | 0 | 0 |
| B\hat{ }2 xdot | 0 | 0 | 1 |
| B C xdot | 0 | 0 | −5.32225 |
| C\hat{ }2 xdot | 0 | 0 | 0 |
| 1 | 0 | 0 | 0 |
| R | 0 | 0 | 0 |
| B | 0.0208276 | 0.0208276 | 0 |
| C | 0.000234706 | 0.000234706 | 0 |
| R\hat{ }2 | 0 | 0 | 0 |
| R B | 0 | 0 | 0 |
| R C | 5.16 | 5.16 | 0 |
| B\hat{ }2 | 0 | 0 | 0 |
| B C | −4.16553 | −4.16553 | 0 |
| C\hat{ }2 | 0.938824 | 0 | 0 |
| R\hat{ }3 | 0 | 0 | 0 |
| R\hat{ }2 B | 0 | 0 | 0 |
| R\hat{ }2 C | 0 | 0 | 0 |
| R B\hat{ }2 | 0 | 0 | 0 |
| R B C | 0 | 0 | 0 |
| R C\hat{ }2 | 20640 | 0 | 0 |
| B\hat{ }3 | 0 | 0 | 0 |
| B\hat{ }2 C | 0 | 0 | 0 |
| B C\hat{ }2 | 0 | 0 | −2.63053 |
| C\hat{ }3 | 0 | 0 | 0 |

As for the Ċ equation, the problem is opposite, i.e., it is the number of missing terms that is too large, leading to non-representative solution.

For results with BIC, the true model is in the collection of models. The collection of models has a total of 21 distinct models. The following are the parameters used when generating data for the model ranking:

| Parameter | Value |
|---|---|
| Number of points per time series | 20 |
| Time of integration | 2 |
| Number of different initial conditions | 100 |
| Percentage of amplitude of added noise (ρ) | 0.0 |
| Rate of missing values in the input data | 0.0 |

Note the small value for the integration time (TIME parameter), larger values make it harder for the model ranking to correctly rank the true model. That is mostly due to the short lived dynamics of the C variable, which very rapidly goes to a steady state given the set of parameters used. Under these parameters the best ranked model is not the true model, it is instead the following model:

| | xdotFalse | xdotTrue | xdot2 |
|---|---|---|---|
| 1 xdot | True | False | False |
| R xdot | True | True | True |
| B xdot | True | True | False |
| C xdot | True | True | False |
| R\hat{ }2 xdot | False | True | False |
| R B xdot | True | True | False |
| R C xdot | True | True | True |
| B\hat{ }2 xdot | True | True | False |
| B C xdot | True | True | True |
| C\hat{ }2 xdot | True | True | True |
| 1 | False | False | False |
| R | True | True | False |
| B | False | False | False |
| C | True | False | False |
| R\hat{ }2 | False | False | False |
| R B | True | True | False |
| R C | True | True | False |
| B\hat{ }2 | False | True | False |
| B C | True | True | False |
| C\hat{ }2 | True | False | False |
| R\hat{ }3 | False | False | False |
| R\hat{ }2 B | False | True | False |
| R\hat{ }2 C | True | True | False |
| R B\hat{ }2 | False | True | False |
| R B C | True | True | False |
| R C\hat{ }2 | True | True | True |
| B\hat{ }3 | False | True | False |
| B\hat{ }2 C | False | True | False |
| B C\hat{ }2 | False | True | True |
| C\hat{ }3 | False | False | True |

The selected model is not sparse, in fact it has a sparsity degree (ratio selected terms) of 0.522. For that reason, it might be interesting to limit the search for the best model only including models that have a sparsity degree smaller than a pre-stablished threshold. If we set this threshold to 30%, in which case the ranking should be called:

no_preprocessing_ids, no_preprocessing_ranks = model_collection.rank(
all_data, max_sparsity=0.3)
best_ranking_model = model_collection[no_preprocessing_ids[0]].

Setting max_sparsity to 0.3 limits the search and allows the system to correctly rank the true model. Even if the noise amplitude and the missing value rate are increased to PERCENTAGE_OF_AMPLITUDE=0.01 and MISSING_VALUE_RATE=0.25, the system still correctly ranks the true model.

### Experiment II—Dynamical System Learner Patient Results

The dynamical system learner (or DynSysLearner) was applied to a series of real data from patients undergoing hemodialysis. The results include data obtained from the application of the method to individual patients, a goal was to look at specific models that were obtained and see their performance when reproducing the input time series through numerical integration. Results were also obtained for the application of the method to a population of patients, aiming to obtain more robust causal information from the statistical analysis of the models obtained from different patients.

The following variables were available for analysis:
prePP: pre-treatment pulse pressure (P).
NLR: Neutrophils-Lymphocytes ratio (ρ_{NL}).
Calcium: Serum calcium concentration (mg/dL) (C_{Ca}).
IntactPTH: Intact Parathyroid Hormone (C_{PTH}).
Albumin: Serum albumin concentration (g/dL) (C_{Ab}).
Phosphorus: Serum phosphorus concentration (mg/dL) (C_{P}).
AP: Alkaline Phosphatase (C_{AP}).

The population included a total of 2558 patients, with average age (62±15) years, average weight (84±24) kg, 57.6% are male, 42.2% are declared white, 40.3% are black, 68 are asian, 25 are native hawaiian/other pacific islander, and 2 are american indian or alaska native.

When applying the method to individual patient data, the following conditions were used:
Using intervals of the input time series with only 1.5 yr time span.
The following steps were taken:
   a) Pre-process the input time series with smoothing spline: —The pre-processing regularization parameter was optimized separately for each variable. —K-fold cross validation with mean squared error (MSE) was used to optimize the regularization parameter. —The pulse pressure is noisier and has a higher sampling rate, for that reason a 5-fold cross validation was used. —All the other variables are more precise and less frequent, so a leave-one-out cross-validation strategy was used.
   b) Define the library of functions with polynomials of order 3, setting the value of max_cross_terms for the denominator to 2:
library.set_numerator_library(polynomial_order=3, max_cross_terms=3) library.set_denominator_library(polynomial_order=3, max_cross_terms=2)
   3. Optimize each variable's regularization parameter within the range [0,0.02] with 100 iterations of the optimizer.
   4. Fine tune the regularization parameters by hand to increase sparsity.

Results were determined for one single randomly selected patient with AP included and three different patients without AP.

For the results with AP, the following table summarizes the values of the regularization parameter and the corresponding sparsity degree for each variable, obtained from the Bayesian optimization:

| | Sparsity | Number | Regularization |
|---|---|---|---|
| | Degree | of terms | Parameter |
| prePP | 0.048780 | 10 | 0.000267 |
| NLR | 0.034146 | 7 | 0.000050 |
| Calcium | 0.014634 | 3 | 0.000800 |
| IntactPTH | 0.058537 | 12 | 0.000300 |
| Albumin | 0.048780 | 10 | 0.000003 |
| Phosphorus | 0.063415 | 13 | 0.000060 |
| AP | 0.073171 | 15 | 0.002126 |

The sparsity degree corresponds to the ratio of the number of selected terms and the length of the input library of functions. In this case, the optimizer was able to successfully find sparse models, with the least sparse model having only 15 terms. The following table depicts the confidence matrix extracted from the obtained model. Each column represents the causal relations present in the model for that particular variable, e.g., if in the column for prePP the row for NLR is True, that means that in the model prePP=ƒ(NLR).

| | prePP | NLR | Calcium | IntactPTH | Albumin | Phosphorus | AP |
|---|---|---|---|---|---|---|---|
| prePP | True | False | False | True | True | True | True |
| NLR | True | True | True | True | True | True | True |
| Calcium | False | True | False | True | True | True | True |
| IntactPTH | True | False | False | True | True | True | True |
| Albumin | False | True | True | True | True | True | True |
| Phosphorus | False | True | False | True | False | False | False |
| AP | True | True | False | True | True | True | True |

For illustrative purposes, the following are the obtained models for each one of the variables:
Pulse pressure:

$$\frac{dP}{dt} = \frac{{P}_{} ( {C}_{AP} , P , {C}_{PTH} , {ρ}_{NL} )}{{P}_{} ( {C}_{AP} , {C}_{PTH} , P )}$$

$${P}_{} = 0.004 {C}_{AP}^{2} - 0.02 {C}_{AP} P + 0.004 {C}_{PTH} P - 1. {ρ}_{NL}^{2} - 1. {ρ}_{NL}^{2} - 1. {ρ}_{NL} P + 0.05 {P}^{2}$$

$${P}_{} = - 0.03 {C}_{AP}^{2} + 0.003 {C}_{AP} {C}_{PTH} + 0.007 {C}_{PTH} P + 0.1 {P}^{2}$$

Neutrophils-Lymphocytes ratio:

$$\frac{d {ρ}_{NL}}{dt} ( {C}_{AP} , {C}_{Ab} , {C}_{Ca} , {ρ}_{NL} , {C}_{P} ) = - 0.0002 {C}_{AP} + 0.0009 {C}_{Ab} + 0.0002 {C}_{Ca} - 0.001 {ρ}_{NL} + 0.002 {C}_{P} + 0.0001$$

Calcium concentration:

$$\frac{{dC}_{Ca}}{dt} ( {C}_{Ab} , {ρ}_{NL} ) = 0.003 {C}_{Ab} - 0.004 {ρ}_{NL}$$

Intact PTH concentration:

$$\frac{{dC}_{PTH}}{dt} = \frac{{PTH}_{} ( {C}_{AP} , {C}_{Ab} , {C}_{Ca} , {C}_{PTH} , {ρ}_{NL} , {C}_{P} , P )}{{PTH}_{} ( {C}_{AP} , {C}_{Ab} , {C}_{Ca} , {ρ}_{NL} , P )}$$

$${PTH}_{} = - 0.01 {C}_{AP} - 0.0009 {C}_{Ab} - 0.003 {C}_{Ca} - 0.002 {C}_{PTH} + 1. {ρ}_{NL} - 0.0008 {C}_{P} - 0.02 P$$

$${PTH}_{} = 0.003 {C}_{AP} + 0.005 {C}_{Ab} + 0.006 {C}_{Ca} - 0.02 {ρ}_{NL} + 0.004 P$$

Albumin Concentration:

$$\frac{{dC}_{Ab}}{dt} = \frac{{Ab}_{} ( {C}_{AP} , {ρ}_{NL} , P , {C}_{Ab} , {C}_{PTH} , {C}_{Ca} )}{{Ab}_{} ( {C}_{AP} )}$$

$${Ab}_{} = 8. \cdot {10}^{- 6} {C}_{AP}^{2} + 9. \cdot {10}^{- 5} {C}_{AP} {ρ}_{NL} - 9. \cdot {10}^{- 6} {C}_{AP} P - 7. \cdot {10}^{- 5} {C}_{Ab} {C}_{PTH} - 2. \cdot {10}^{- 5} {C}_{Ab} P + 3. \cdot {10}^{- 5} {C}_{Ca} {C}_{PTH} - 4. \cdot {10}^{- 6} {C}_{Ca} P - 6. \cdot {10}^{- 6} {C}_{PTH} {ρ}_{NL} + 4. \cdot {10}^{- 5} {ρ}_{NL} P$$

$${Ab}_{} = {C}_{AP}$$

Phosphorus Concentration:

$$\frac{{dC}_{P}}{dt} = \frac{{P}_{} ( {C}_{AP} , P , {C}_{PTH} , {C}_{Ab} , {C}_{Ca} , {ρ}_{NL} )}{{P}_{} ( {C}_{AP} , {ρ}_{NL} , {C}_{PTH} , P )}$$

$${P}_{} = - 0.0002 {C}_{AP}^{2} P - 0.0001 {C}_{AP} {C}_{PTH} P - 0.0002 {C}_{AP} {P}^{2} - 0.0002 {C}_{Ab} {C}_{PTH}^{2} - 6. \cdot {10}^{- 5} {C}_{Ca} {C}_{PTH}^{2} + 0.0001 {C}_{PTH}^{2} {ρ}_{NL} + 0.009 {C}_{PTH} {ρ}_{NL} P - 0.0002 {C}_{PTH} {P}^{2} + 8. \cdot {10}^{- 5} {P}^{3}$$

$${P}_{} = 1. {C}_{AP}^{2} {ρ}_{NL} + 0.0001 {C}_{AP} {C}_{PTH}^{2} + 0.0006 {C}_{PTH}^{2} P + 0.01 {C}_{PTH} {P}^{2}$$

AP Concentration:

$$\frac{{dC}_{AP}}{dt} = \frac{{AP}_{} ( {C}_{AP} , {C}_{PTH} , P , {C}_{Ab} , {ρ}_{NL} , {C}_{Ca} )}{{AP}_{} ( {C}_{AP} , {C}_{PTH} , {C}_{Ab} , {C}_{Ca} , P )}$$

$${AP}_{} = 0.005 {C}_{AP}^{2} - 0.007 {C}_{AP} {C}_{PTH} + 0.01 {C}_{AP} P - 0.03 {C}_{Ab} {C}_{PTH} - 0.06 {C}_{Ca} {C}_{PTH} + 0.3 {C}_{PTH} {ρ}_{NL} - 0.005 {ρ}_{NL} P - 0.008 {P}^{2}$$

$${AP}_{} = - 1. {C}_{AP}^{2} + 0.1 {C}_{AP} {C}_{PTH} + 0.01 {C}_{Ab} {C}_{PTH} + 0.02 {C}_{Ca} {C}_{PTH} - 0.004 {C}_{PTH}^{2} + 0.04 {C}_{PTH} P + 0.05 {P}^{2}$$

For the results without AP, The exclusion of AP could improve the quality of the obtained models for two reasons: (i) by reducing the number of equations in the system, (ii) and because AP has a more indirect influence in the physiology of vascular calcification.

For Patient 1, In the following tables there are the results for the sparsity degree and regularization parameter obtained from the Bayesian optimizer with 10 iterations. Originally the value of the regularization parameter obtained for the NLR equation was not sufficiently low to enforce the desired degree of sparsity. For that reason, it was artificially decreased.

| | Sparsity | Number | Regularization |
|---|---|---|---|
| | Degree | of terms | Parameter |
| prePP | 0.074324 | 11 | 0.003972 |
| NLR | 0.054054 | 8 | 0.020000 |
| Calcium | 0.081081 | 12 | 0.007363 |
| IntactPTH | 0.054054 | 8 | 0.013704 |
| Albumin | 0.101351 | 15 | 0.013526 |
| Phosphorus | 0.081081 | 12 | 0.000060 |

The next table shows the confidence matrix obtained from the model.

| | Phosphorus | Calcium | IntactPTH | Albumin | NLR | prePP |
|---|---|---|---|---|---|---|
| Phosphorus | True | True | True | True | True | True |
| Calcium | True | True | True | True | True | True |
| IntactPTH | False | True | False | True | False | False |
| Albumin | True | True | True | True | True | True |
| NLR | True | True | True | True | True | True |
| prePP | True | False | False | True | False | True |

The following are the associated equations:
Pulse Pressure:

$$\frac{dP}{dt} = \frac{- 0.04 {C}_{Ab} + 0.08 {C}_{Ca} + 0.2 {ρ}_{NL} - 0.1 {C}_{P} - 0.008 P}{0.2 {C}_{Ab} + 0.2 {C}_{Ca} + 1. {ρ}_{NL} - 0.6 {C}_{P} - 0.06 P + 0.04}$$

Neutrophils-Lymphocytes ratio:

$$\frac{d {ρ}_{NL}}{dt} = \frac{- 3. {C}_{Ab} + 1. {C}_{Ca} - 0.9 {ρ}_{NL} + 0.5 {C}_{P} + 2.}{4. {C}_{Ab} + 0.2 {C}_{Ca} + 0.2 {ρ}_{NL}}$$

Calcium concentration:

$$\frac{{dC}_{Ca}}{dt} = \frac{{Ca}_{} ( {C}_{Ab} , {C}_{PTH} , {ρ}_{NL} , {C}_{P} , {C}_{Ca} )}{{Ca}_{} ( {C}_{PTH} , {C}_{Ab} , {C}_{Ca} , {ρ}_{NL} )}$$

$${Ca}_{} = - 3. {C}_{Ab} {C}_{PTH} + 0.01 {C}_{Ab} {ρ}_{NL} + 0.2 {C}_{Ab} {C}_{P} + 1. {C}_{Ca} {C}_{PTH} + 0.03 {C}_{Ca} {ρ}_{NL} - 0.1 {C}_{Ca} {C}_{P} - 1. {C}_{PTH} {ρ}_{NL} + 0.5 {C}_{PTH} {C}_{P} + 5. {C}_{PTH} {C}_{P} + 5. {C}_{PTH}$$

$${Ca}_{} = {C}_{PTH} ( 5. {C}_{Ab} + 0.2 {C}_{Ca} + 0.2 {ρ}_{NL} )$$

Intact PTH concentration:

$$\frac{{dC}_{PTH}}{dt} = \frac{- 2. {C}_{Ab} + 0.7 {C}_{Ca} + 3. {ρ}_{NL} - 1. {C}_{P} - 2.}{0.3 {C}_{Ca} - 0.2 {ρ}_{NL} + 0.1 {C}_{P}}$$

Albumin Concentration:

$$\frac{{dC}_{Ab}}{dt} = \frac{{Ab}_{} ( {C}_{Ca} , {ρ}_{NL} , P , {C}_{P} )}{{Ab}_{} ( {C}_{Ab} , {C}_{PTH} , {C}_{Ca} , {ρ}_{NL} , {C}_{P} )}$$

$${Ab}_{} = 1. {C}_{Ca}^{2} - 0.9 {C}_{Ca} {ρ}_{NL} - 0.4 {C}_{Ca} P - 0.2 {ρ}_{NL} P + 0.2 {C}_{P} P + 0.04 {P}^{2}$$

$${Ca}_{} = {C}_{PTH} ( 5. {C}_{Ab} + 0.2 {C}_{Ca} + 0.2 {ρ}_{NL} )$$

Phosphorus Concentration:

$$\frac{{dC}_{P}}{dt} = - \frac{0.0001 {C}_{Ab} {C}_{P}}{P} - 0.003 {C}_{Ab} + \frac{0.0001 {C}_{Ca}^{2}}{P} + \frac{0.0002 {C}_{Ca} {ρ}_{NL}}{P} - 0.002 {C}_{Ca} + \frac{0.0002 {ρ}_{NL}^{2}}{P} - \frac{0.0001 {ρ}_{NL} {C}_{P}}{P} + 0.005 {ρ}_{NL} - 0.001 {C}_{P} + 0.0002 P - 0.0005$$

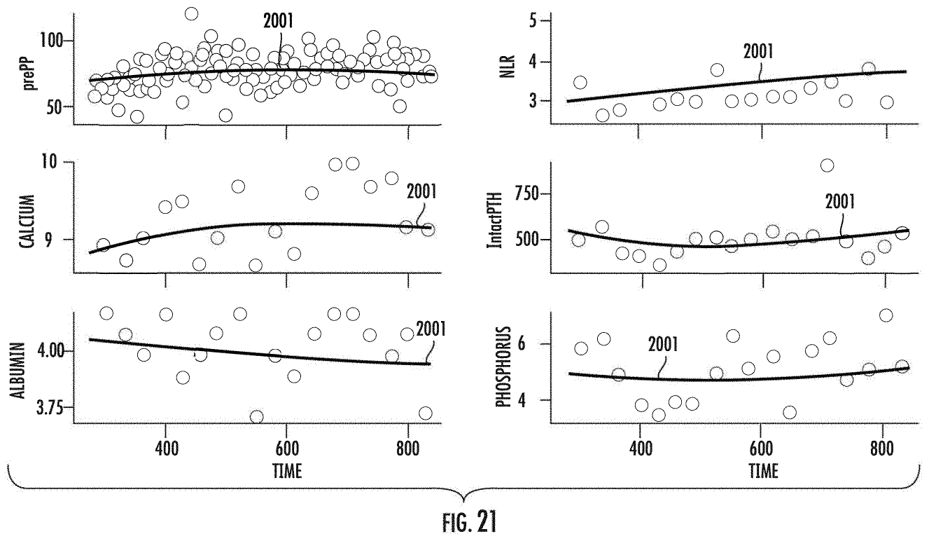

In order to validate the obtained models, it is possible to numerically integrate the equations and compare the solution with the original data. As initial condition, it was used the value of the variables pre-processing at t=0. FIG. 21 provides graphs of the original data (dots), the original pre-processing and the integrated solution from the obtained equations. For FIGS. 21-24, the 2000 lines are the original lines and the 2001 lines are the lines using processes according to some embodiments (if only a 2001 line is depicted, the 2001 line completely overlapped the corresponding 2000 line). As can be seen, the 2001 lines overlap the 2000 lines perfectly, meaning that the obtained equations perfectly describe the original pre-processed data.

An important remark is that the entire time series was used as input data of the dynamical system learner (DynSysLearner or DynSysLearnerND), in order to more accurately test the obtained equations, it is necessary to test their efficiency against a section of the time series that was not used as input.

The following table provides the results of the regularization parameter and sparsity degree for patient 2, the Bayesian optimizer was used with only 10 iterations:

| | Sparsity | Number | Regularization |
|---|---|---|---|
| | degree | of terms | parameter |
| prePP | 0.128378 | 19 | 0.087622 |
| NLR | 0.168919 | 25 | 0.002156 |
| Calcium | 0.047297 | 7 | 0.049756 |
| IntactPTH | 0.243243 | 36 | 0.060819 |
| Albumin | 0.060811 | 9 | 0.027989 |
| Phosphorus | 0.141892 | 21 | 0.051091 |

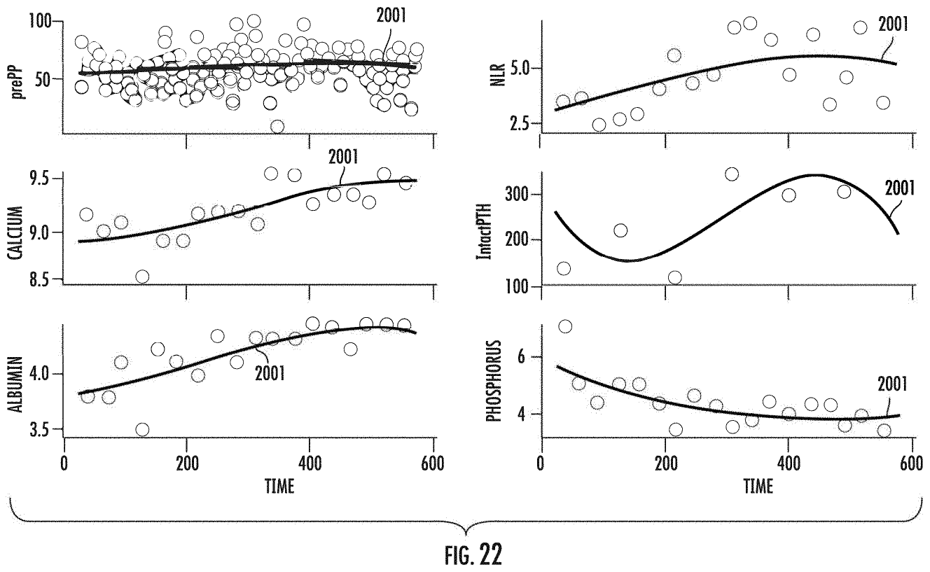

FIG. 22 illustrates an integrated time series plotted with the original data and original pre-processing. Interestingly, patient 2 presents a richer dynamics than the one observed in patient 1, and the DynSysLearnerND is still able to find a set of equations that successfully reproduce the system dynamics for the same period of time provided as input.

For patient 3, the results are presented in the following table:

| | Sparsity | Number | Regularization |
|---|---|---|---|
| | degree | of terms | parameter |
| prePP | 0.182432 | 27 | 1.656041e−01 |
| NLR | 0.040541 | 6 | 2.273460e−01 |
| Calcium | 0.054054 | 8 | 2.393089e−01 |
| IntactPTH | 0.189189 | 28 | 7.294742e−02 |
| Albumin | 0.918919 | 136 | 2.882139e−13 |
| Phosphorus | 0.216216 | 32 | 5.882389e−02 |

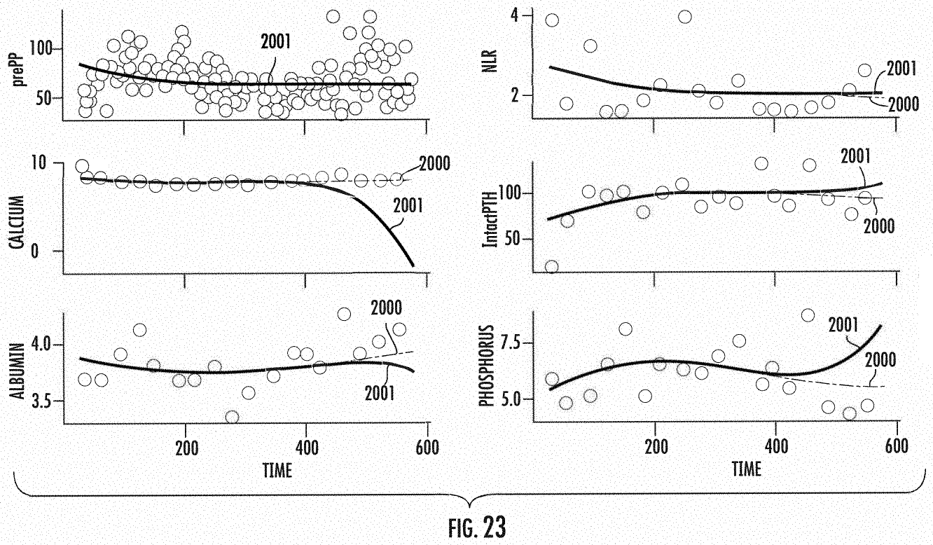

In this case, the optimizer was unable to find an appropriate value for the regularization parameter for the equation for Albumin. consequently, the number of terms in the obtained equation was 136 (with a sparsity degree of 0.92). FIG. 23 depicts graphs of the results from the integration of the equations, for a time larger than ˜400 days, the integrated equation start to diverge from the pre-processed time series, which suggests that non-sparse models are less stable, even for time intervals that were used is input for the method.

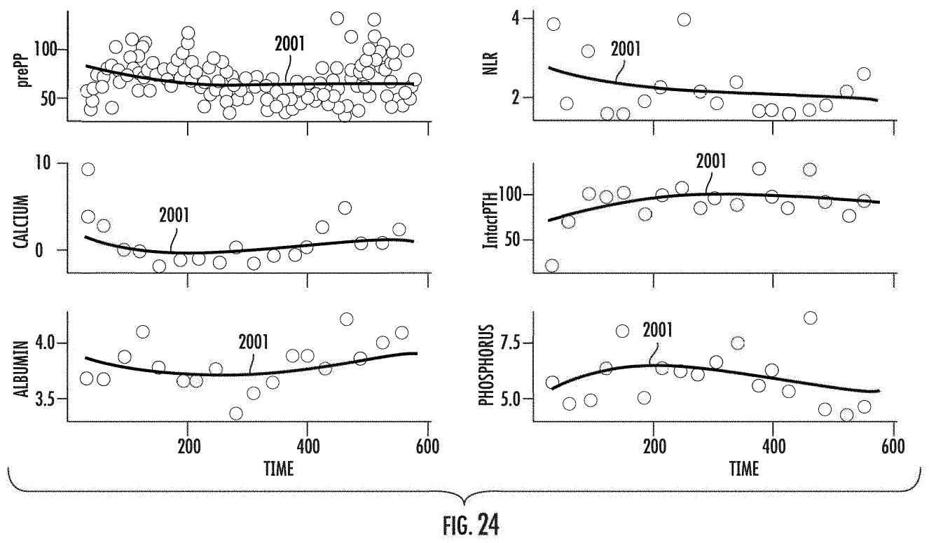

In order to increase sparsity in the obtained model, the DynSysLearner can also run with regularization parameters informed by the user (in which case the Bayesian optimizer is not used). Using pre-determined regularization parameters (informed in the following table), the method is capable of finding more sparse equations (0.095 sparsity degree for the albumin equation), which leads to more stable solutions of the obtained equations, as shown in the graphs of FIG. 24.

| | Sparsity | N Number | R regularization |
|---|---|---|---|
| | degree | of terms | parameter |
| prePP | 0.141892 | 21 | 0.003972 |
| NLR | 0.121622 | 18 | 0.020000 |
| Calcium | 0.114865 | 17 | 0.007363 |
| IntactPTH | 0.074324 | 11 | 0.013704 |
| Albumin | 0.094595 | 14 | 0.013526 |
| Phosphorus | 0.175676 | 26 | 0.000060 |

In order to analyze the performance of the system over a population of patients, a smaller subpopulation was selected from the original one with 2558 patients. The inclusion criteria was patients that had at least 1.5 years of data and did not present any of the following comorbidities: Ischemic heart disease, Peripheral vascular or arterial disease, Congestive heart failure, Cardiovascular disease, Chronic obstructive pulmonary disease, Myocardial infarction including cardiac arrest comorbidity, Hypertension, Cardiac dysrhythmia, and Diabetes.

A total of 129 patients were selected that satisfied all of these conditions, with average age (57±17) years, average weight (80±25) kg, of which 40.3% are white, 36.4% are black, and 9 are Asian. The regularization parameters were fixed with the same value for every patient in the population:

```
REGULARIZATION_PARAMETER = {
  ″prePP″: 0.003972,
  ″NLR″: 0.02,
  ″Calcium″: 0.007363,
  ″IntactPTH″: 0.013704,
  ″Albumin″: 0.013526,
  ″Phosphorus″: 0.000060,
}
```

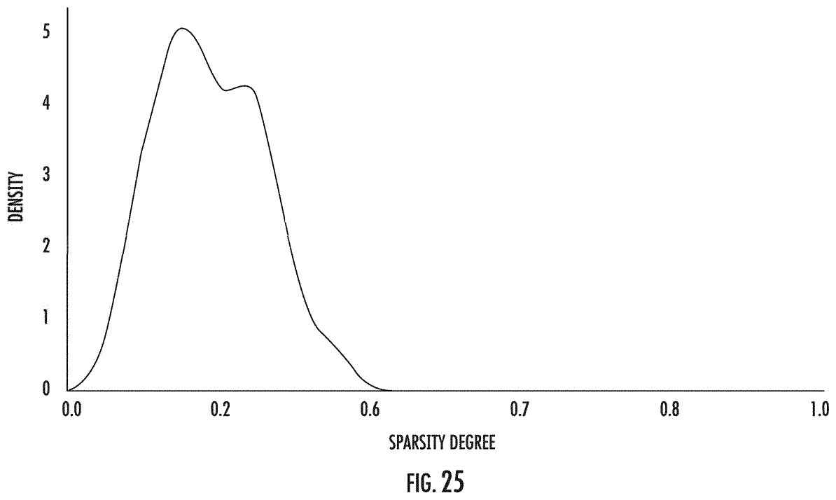

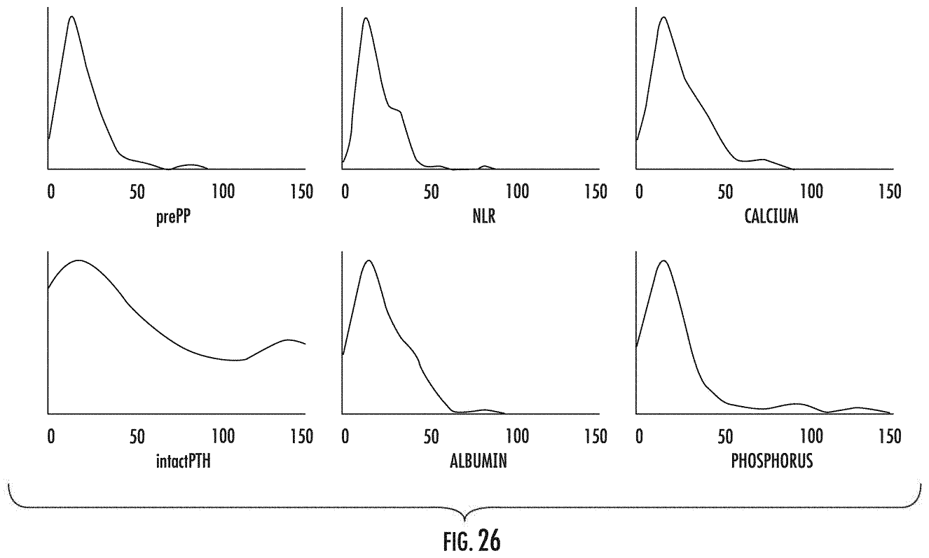

The validation of the value of the regularization parameter can be made indirectly by evaluating the sparsity degree of the obtained models. FIG. 25 depicts a graph of the probability distribution function (density) of the sparsity degree of the full model for the entire subpopulation. Even though there are models that have relatively high sparsity degrees (˜0.4), the majority has values of sparsity degree smaller than 0.3. In order to have a better understanding of how each variable contributes to the sparsity of the selected mode, FIG. 26 depicts graphs of the distribution of the number of terms for each one of the variables. For all the variables, there were a few selected models that were non-sparse, which can be seen by a small peak between 50 and 100 terms present in all the variables' distribution. However, the majority of models selected had under 50 terms selected. Exceptionally, the DynSysLearner was unable to find a sparse model for the IntactPTH for the majority of models.

From the selected models, it is possible to perform a causal analysis that is averaged over the entire population and extracts more robust causal information between the variables.

According to some embodiments, the causal diagrams were obtained in two different ways:
From the probability of term: from all the selected models of the entire population it was built a probabilistic model, which represents what is the probability that a given term from the library of functions was selected in that population. By thresholding this probabilistic model, i.e., by selecting only terms with probability of occurrence larger than a pre-established threshold, it is possible to have a final model that represents the population and from such model extract the associated confidence matrix;
From the probability of the confidence matrix: in this approach, for every patient of the population it was built a confidence matrix from the selected model. The confidence matrices from all of the patients were used to build a probabilistic confidence matrix, i.e., each elements corresponds to the probability that a given causal link was present in that population. By thresholding this probabilistic confidence matrix, it is then possible to build causal diagrams for the entire population.

From the two approaches there is a slight difference in interpretation. The first approach represents the probability that a given term was selected, therefore it is more restrictive in the sense that it counts not only the existence of a causal link, but that the causal relationships have a similar functional form for different patients. On the other hand, the second approach is less restrictive as it only takes in consideration the existence of a causal dependency between two variables, regardless of its functional form.

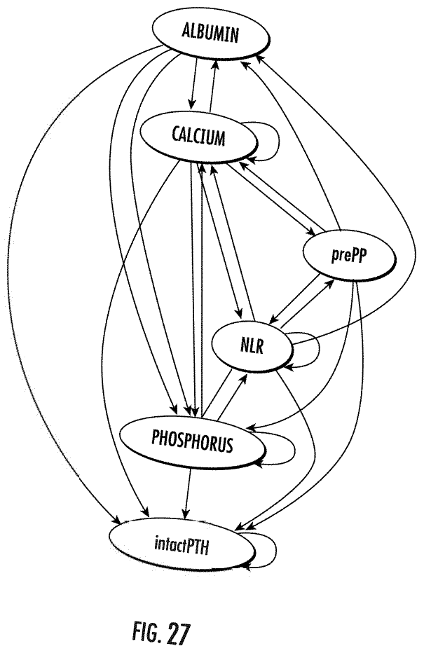

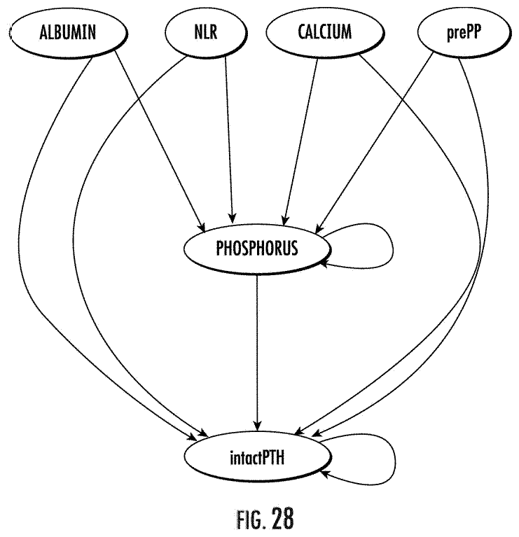

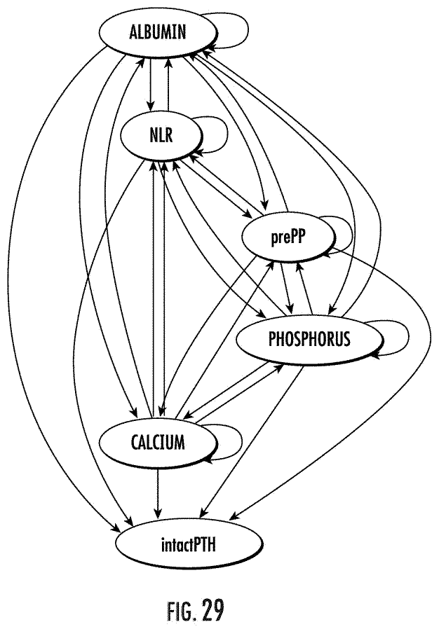

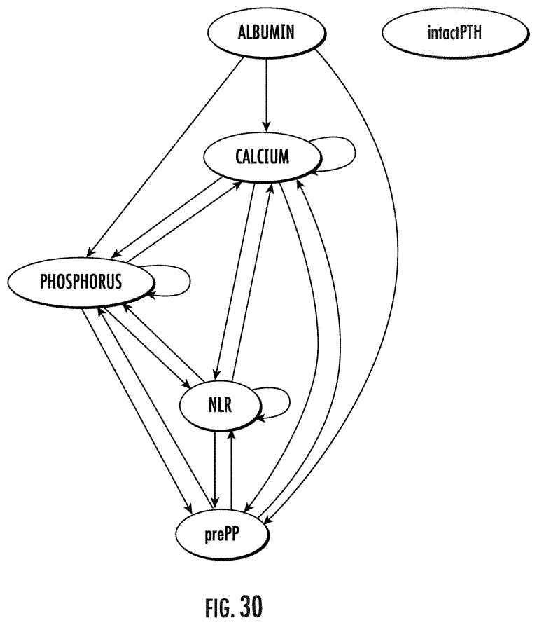

FIGS. 27 and 28 depict causality diagrams obtained from the probability of terms with thresholds equal to 0.4 and 0.5, respectively. FIGS. 29 and 30 depict causality diagrams obtained from the probability of the confidence matrix with thresholds 0.80 and 0.95, respectively.

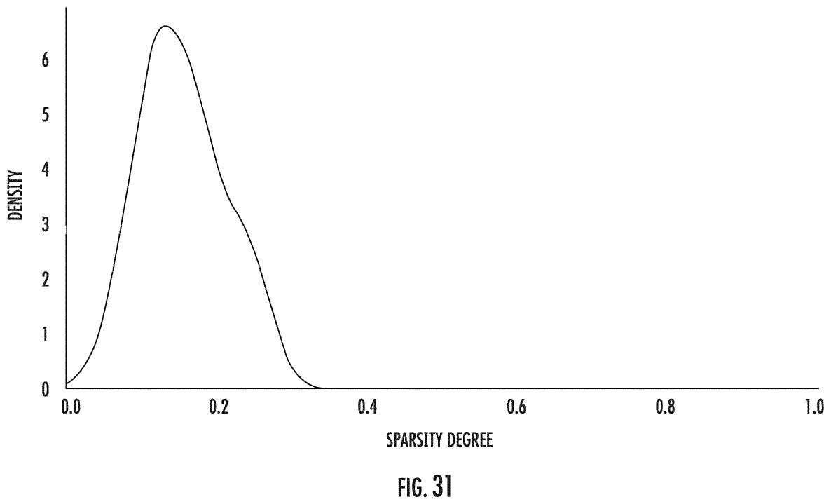

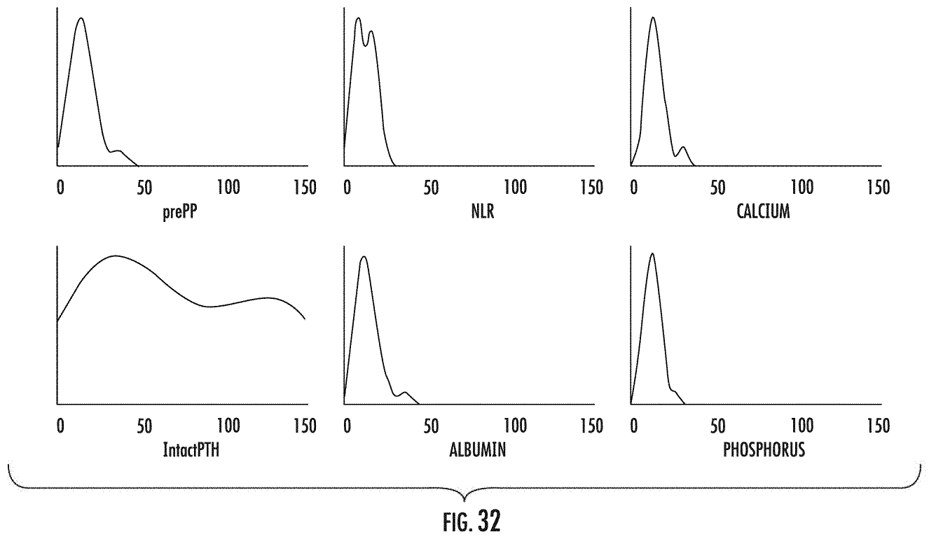

The same analysis from the previous section was performed on a subpopulation with only 25 randomly selected patients, in this case the regularization parameters were optimized with a Bayesian optimizer with 100 iteration. FIGS. 31 and 32 depict the density of the sparsity degree and number of terms of separated variables, respectively. In comparison with the results with pre-determined regularization parameters, the obtained models are sparser, even though the models selected for Intact PTH still present a high probability of being non-sparse.

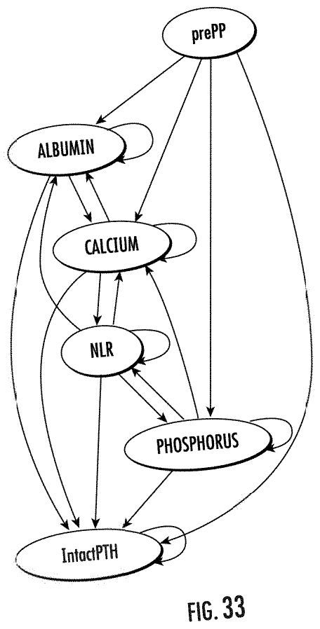

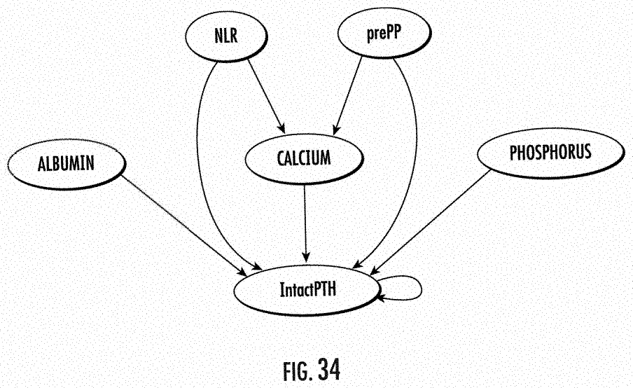

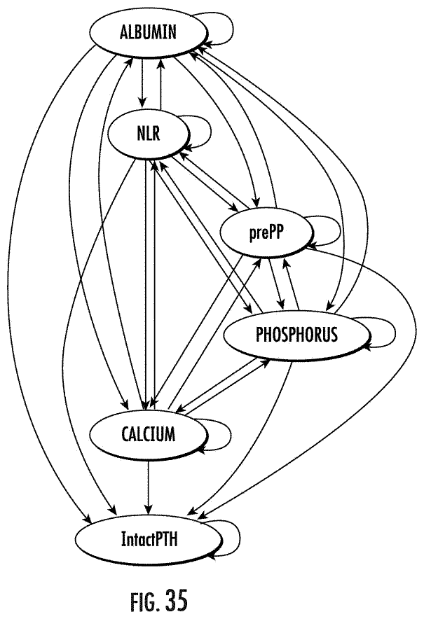

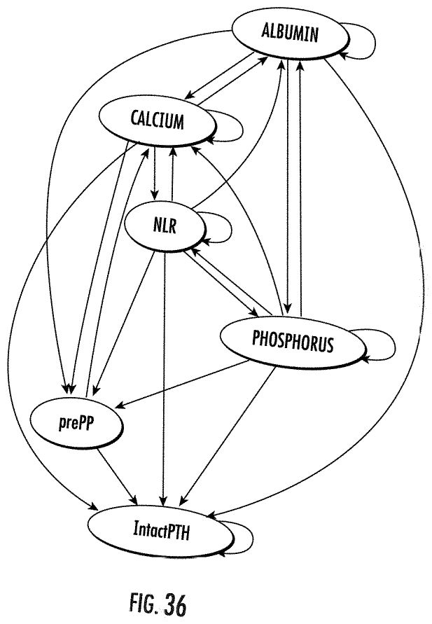

FIGS. 33 and 34 depict causality diagrams obtained from the probability of terms with thresholds equal to 0.4 and 0.5, respectively. FIGS. 35 and 36 depict causality diagrams obtained from the probability of the confidence matrix with thresholds 0.80 and 0.95, respectively.

### Experiment III—Dynamical System Modeler Implementation

A dynamical system modeler process may be implemented using various systems, platforms, and/or the like. For example, this Example Implementation is written in Python 3 and is structured around a Patients class, which organizes and handles patients data. The raw data may be in various forms, including, without limitation, a *.csv or other table file, for instance, where each column represents a different variable and each row is a different measure of those variables, at different times. There may be other data columns, rows, fields, and/or the like. For example, the patient data may include a column providing patient identification.

The Patients class may be structured around, for example, the Pandas multi-index data frame as described in McKinney, W., “Data Structures for Statistical Computing in Python,” Proceedings of the 9th Python in Science Conference, pp. 56-61 (2010). In some embodiments, the Patients class may include the following attributes: data: data frame with the raw data passed to the class; IDS: list of numbers with the patients' identification; npatients: an integer variable storing the total number of patients; variables: list with the column names of the variables in the study; metadata: data frame with each patient metadata: duration of the study, date of death, and/or the like; nobs: data frame with the number of observations for each variable and every patient; and parameters: values for the criteria used to select valid patients.

In some embodiments, the Patients class may include the following methods: read_raw_data: read the data from a data frame without any pre-processing (raw format)l; select_valid: select only the valid patients (invalid ones are dropped), where validity is determined by the total and a yearly number of observations; drop_borders: drop patient data from the borders of the time series; addpatient: add new patient information to the dataset; delpatients: remove patients from the dataset based on the list of IDS; to_file: write patients' information to h5 file; from_file: read patients' information from the h5 file; sort_IDS: sort the patients' IDs; and get_from_IDS: get the patient information directly from ID.

The input data is noisy and non-uniform, where non-uniformity in this context means that the time step between successive points is not constant throughout the time series. Accordingly, the time series is pre-processed. The pre-processing is managed by the class preProcessPopulation which applies one of the different pre-processing methods implemented to all the patients in a population stored in a Patients class object.

The preProcessPopulation class has three methods: setup: reads an object derived from the preProcessing base class. This object implements a particular pre-processing method; read_patients: reads an instance of the Patients class; and run: a method that performs the actual pre-processing, it must be called after the two previous methods.

The preProcessing class is used as the base for all the objects that implement a particular pre-processing, it provides the interface for two methods: process and verify. The process method reads the data from a particular patient and performs the actual pre-processing. Every object must have its own version of it. The verify method is called by the preProcessPopulation before it starts the pre-processing, its intent is simply verifying if all the necessary configuration was called prior to the calculation. If the child class does not implement it, the preProcessing class provides it.

Non-limiting examples of classes implemented for pre-processing may include: preProcessingDoNothing: which simply smooths the variables marked as noisy, for all the other variables it does nothing; preProcessingSpline: applies a simple spline to the data; preProcessingGP: uses Gaussian process as pre-processing; and preProcessingKNN: applies the nearest neighbors algorithm to impute the missing data.

The following code excerpt exemplifies the general workflow for pre-processing using splines.

import ckdmbdvc as mb
import ckdmbdvc.preprocessing as mbp
noisyvars = [...]
pop = mb.Patients( ).from_file(...)
spline = mbp.preProcessingSpline( )
population_pre_process = mbp.preProcessPopulation( )
population_pre_process.read_patients(pop)
population_pre_process.setup(spline)
population_pre_process.smooth_frequent
(noisyvars, timewindow=′month′, method=′gp′)
population_pre_process.run(dt=0.5, processes=NPROCESSES)
NewpopS = population_pre_process.NewPop
NewPopS.to_file(...)

It is possible to average the result of two different pre-processing steps, which is done through the average_population function from the preprocessing module. Two different methods to calculate the weights of the averages are implemented: linear and exponential. The usage of the average is exemplified as follows:

import ckdmbdvc as mb
original_data = mb.Patients( ).from_file(...)
pre_processing_1 = mb.Patients( ).from_file(...)
pre_processing_2 = mb.Patients( ).from_file(...)
average = mb.preprocessing.average_population( original_data,
(pre_processing_1, pre_processing_2), method='...'),

where the method parameter should be one of linear or exp.

The implementation of statistical analysis follows a similar structure to that of the pre-processing. It is structured around two classes: AnalysisManagerBase, which is a base class for different statistical or causal analysis one might want to implement; and PopulationAnalyzer, which applies any method to a Patients class instance.

The AnalysisManagerBase provides an interface for any child class that implements any statistical test to be applied to the Patients class. It enforces two methods: process, which must be provided by the child class, and that reads a data frame with the patient data; and verify, which is supposed to verify any configuration required by the method implemented. The base class provides a verify method that does nothing, in case the implemented method has no requisites.

There are two different correlation tests implemented: Kendall and Spearman correlation (see, for example, Kendal et al., “A New Measure of Rank Correlation,” Biometrika, 30(1-2), pp. 81-93 (1938) and Kokoska et al., CRC Standard Probability and Statistics Tables and Formulae, Student Edition. Taylor & Francis (2000)), which are available through the classes KendallCorrelationManager and SpearmanCorrelationManager, respectively. The following shows how to calculate Kendall correlation for a particular population of patients:

```
population = mb.Patients( ).from_file(...)
kendall_manager = mbs_KendallCorrelationManager( )
analyzer = mbs.PopulationAnalyzer( )
analyzer.setup(kendall_manager)
analyzer.read_patients(population)
analyzer.run( )
analyzer.results.to_parquet(″path to .parquet file″)
mbs.correlation_heatmap(
  mbs.KendallCorrelationManager.average_over_population(
    analyzer.results),
  filename=″path to .png filename″)
```

The results are stored in a multi-index data frame at analyzer.results. Both classes provide an average_over_population static method, which averages the correlations calculated for each patient. The correlation_heatmap function is a wrapper that plots the heatmap from matplotlib for the calculated correlations.

The causality analyses are also implemented as classes derived from AnalysisManagerBase, namely: GrangerCausalityManager and KernelGrangerCausalityManager, for the Granger causality and kernel Granger causality, respectively. Both classes share a similar structure (as they are both derived from the same parent class), only the kernel Granger causality has a method get_causality_metadata implemented that is responsible for calculating the values of hyperparameters of the method, and it should be called right after the class in instantiated. The following code shows how to use the KernelGrangerCausalityManager class:

population = mb.Patients( ).from_file( )
granger_manager = mbs.KernelGrangerCausalityManager(...)
granger_manager.get_causality_metadata(...)
analyzer = mbs.PopulationAnalyzer( )
analyzer.setup(granger_manager)
analyzer.read_patients(population)
confidence = analyzer.run( )
number_of_patients = mbs.how_many_patients(confidence)
confidence.to_pickle(″path to .pickle file″)
population_confidence = mbs.create_confidence_matrix_
for_population(confidence)
G = mbs.create_graph_from_confidence_matrix
(population_confidence, voting_threshold)
mbs.generate_matplotlib_figure_of_graph(G, figurename)
mbs.generate_agraph_figure(G, figurename).

The code is implemented for separate data only, i.e., given a set of data for X and \dot{X} (where X is a matrix where each column corresponds to the time-series of a different variable), the algorithm returns a set of coefficients for each element of a given pre-established function library.

The library of functions is defined through the class LibraryConstructor, this object should be instantiated before the call of select_best_model. The following provides exemplary usage:

from ckdmbdvc.modeling.selection import (
LibraryConstructor, select_best_model, sympyfy_model)
library = LibraryConstructor(polynomial_order=order)
model = select_best_model(X, Xdot, library)
symb_model = sympyfy_model(model).

Where order is the maximum order of the polynomial terms in the library of functions, X and Xdot are the time series and their derivatives, respectively. Model is a data frame where each column corresponds to a different variable and each row to a different term in the library of functions, the value of each element corresponds to the coefficient of that term in the selected model. The function sympyfy_model transforms the data frame representation of the selected model into a list of sympy expressions.

A potential source of error from the causality analysis is its dependency on the imputation method used to handle the missing data values. A possible approach to overcome this difficulty is to use a probabilistic approach derived from Gaussian processes. Given a data matrix X where each column corresponds to the time series of a different variable of your system, and Σ is the matrix of all their standard deviations, and a Kernel function K; let [symbol] (X, Σ, K) be the Gaussian process derived from X and K in a way that [symbol] ˜ [symbol] (X, Σ, K) is a random time series sampled from [symbol] .

Each different sample drawn from the GP, when subjected to the Kernel Granger causality method, results in a different confidence matrix. Consequently, a causal link, let us say from xᵢ→xⱼ, might not be present in the confidence matrix from all the samples. Based on the frequency of selection of a given causal link over multiple samples, it is possible to build the probability P_{i→j} that variable xᵢ causes xⱼ.

Given an appropriate choice of the kernel function K, and the correct estimation of the variables' standard deviation Σ, the samples from the Gaussian process are all valid models for the time series, given the information available. In this regard, the replacement of a deterministic view on causality by a probabilistic one directly handles the problem of the validity of the chosen imputation/modeling method used.

To use Granger-based methods to identify causal relations in deterministic non-linear systems might be problematic because these methods rely on the dynamics of the variables of the system being separable, which is not always true for non-linear systems. For this reason, special care must be taken when looking for causal relations in such systems.

One interpretation for causality is that if changes in xᵢ lead to changes in xⱼ, then xᵢ is said to cause xⱼ. Possibly, this relationship could be estimated by measuring how much information is shared by xᵢ and the derivative of xⱼ. Assuming mutual information as a valid measure for this goal, we could define the Boolean variable [symbol] (i→j) as being true if xᵢ causes xⱼ, under the condition:

[symbol]

(i→j)=μ(xᵢ,\dot{x}ⱼ)>ϵ

where μ(x, y) is the mutual information between x and y, and ϵ is a pre-established threshold. The use of mutual information (or any other metric of shared information between the variables and the derivatives) can help reduce the number of terms in the library of functions in the model selection algorithm, which could greatly reduce its time of execution.

The method includes calculation of the sparse regression on the terms of the function library. To that end, several different methods can be used, including, without limitation: STLSQ: sequential thresholding least square, which performs repeated linear regression on the data, removing from the following iterations the terms with the absolute value of the coefficients smaller than a given threshold; EFS: evolutionary feature synthesis, a regression technique on features based on evolutionary computation; FFX: fast function extraction, a symbolic regression technique; STRidge: sequential threshold ridge regression, which is similar to STLSQ, but it uses a ridge regression instead of a linear regression.

The method of model selection may include the discovery of systems with some spatial dependency, in which case a variable of interest (ψ) will depend not only on time t, but also on some spatial variable x, therefore ψ=ψ(t, x). The mathematical formulation of this problem is not fundamentally different from the model selection algorithm presented for the case in which ψ is only time dependent. However partial differential equation recovery from noisy data seems to be more sensitive to the noise magnitude, making the denoising step of the process of paramount importance.

Let ψ(t, \vec{r}) be the real value of the variable of interest, where \vec{r}∈ [symbol] ⁿ is a vector of the spatial coordinates, where n can be any value, but will generally be limited to 1, 2, or 3. It is assumed that the spatiotemporal evolution of ψ(t, \vec{r}) is given by:

[symbol]

(ψ,ψₜ,ψ_{\vec{r}},ψ_{\vec{r}\vec{r}}, . . . ,\vec{r},t)=0

Where [symbol] is an operator on ψ and its derivatives. [symbol] is supposed to be linear on a small number of terms, e.g., the Korteweg-De Vries equation [symbol] is a linear combination of the terms ψₜ, ψₓₓₓ and ψψₓ, so that the KdV equation is given by: ψₜ+ψₓₓₓ−6ψψₓ=0. If noisy data ϕ is measured at different locations of t and \vec{r}, and:

ϕ(t,\vec{r})=(t,\vec{r})+σ(t,\vec{r})

where σ is the noise amplitude, which might depend on the spatial location of the measured point. Let [symbol] (⋅, nₜ, nₓ) be a denoising operator, which could be a Gaussian process (Rasmussen and Williams, 2005), or some machine learning-based algorithm, and nₜ and nₓ are the orders of desired time and space derivatives, respectively. In a way that the application of [symbol] on ϕ allows the recovery of an approximation of ψ and its derivatives, denoted \tilde{ψ}, i.e., ψ≈\tilde{ψ}= [symbol] (ϕ, nₜ=0, nₓ=0).

With the denoising operator at hand, it is possible to build a function library Λ, which may be or may include a matrix where each column is a different term of the library, and each row corresponds to a different time and space position from the data:

$$Λ = ( \begin{matrix}\tilde{ψ} ( {t}_{0} , {x}_{0} ) & {\tilde{ψ}}_{t} ( {t}_{0} , {x}_{0} ) & … \\ ⋮ & ⋮ & … \\ \tilde{ψ} ( {t}_{N} , {x}_{0} ) & {\tilde{ψ}}_{t} ( {t}_{0} , {x}_{0} ) & … \\ ⋮ & ⋮ & … \\ \tilde{ψ} ( {t}_{i} , {x}_{j} ) & {\tilde{ψ}}_{t} ( {t}_{0} , {x}_{0} ) & … \\ ⋮ & ⋮ & … \\ \tilde{ψ} ( {t}_{N} , {x}_{M} ) & {\tilde{ψ}}_{t} ( {t}_{0} , {x}_{0} ) & …\end{matrix} )$$

In this way, the model selection algorithm is reduced to finding the sparse solution \vec{ξ} to the following equation:

Λ\vec{ξ}=0

where \vec{ξ} is a vector with the coefficients of each term in the function library.

Delay differential equations are ordinary differential equations in which the current value of the derivative depends on past as well as current values of the state variables, i.e.:

\vec{x}=\vec{ƒ}(t,\vec{x},\vec{x}_{τ})

where \vec{x}_{τ}(t)=\vec{x}(t−τ). Its formulation is entirely like the formulation of the original model selection algorithm, with the difference that the library of functions should be extended with terms from τ times before, i.e., let Λₜ be the original library of functions at time t, the new extended library \tilde{Λ}ₜ is given by:

\tilde{Λ}ₜ=(ΛₜΛ_{t-τ})

The determination of the appropriate value of τ can be made from different methods: (i) it can be any typical time scale derived from the underlying problem; (ii) it can be derived from the first minimum of the autocorrelation function or from the delayed mutual information.

The applications of the CKD/ESRD condition analysis process, such as a vascular calcification analysis process, described in the present disclosure are not limited to the study of vascular calcification on patients with CKD, but can be extended to the study of any system in which there is access to time series data. One non-limiting application may include the development of virtual clinics. Different from the usual machine learning models, in which a prediction from a target variable is made based on input. The method proposed allows the identification of a model that explains the relationship between the studied variables and allows for simulation and numerical experimentation. With such a model at hand, it is possible to simulate the effect of different interventions, which can provide insight into different courses of action for treatment.

More importantly, because the model is derived directly from the data set, it is possible not only to generate a general model with scientific interest, which can be used to better understand a given phenomenon. But it is also possible to determine a model-specific for a particular data set (from one patient, for example), which could provide information tailored for that particular patient, instead of a model derived from average behavior from a given population of patients.

### Experiments: Dynamical System Modeler Process—Model Discovery

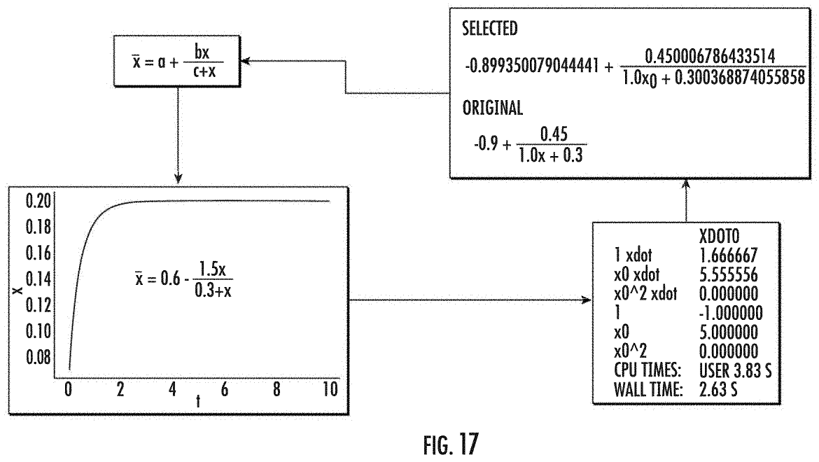

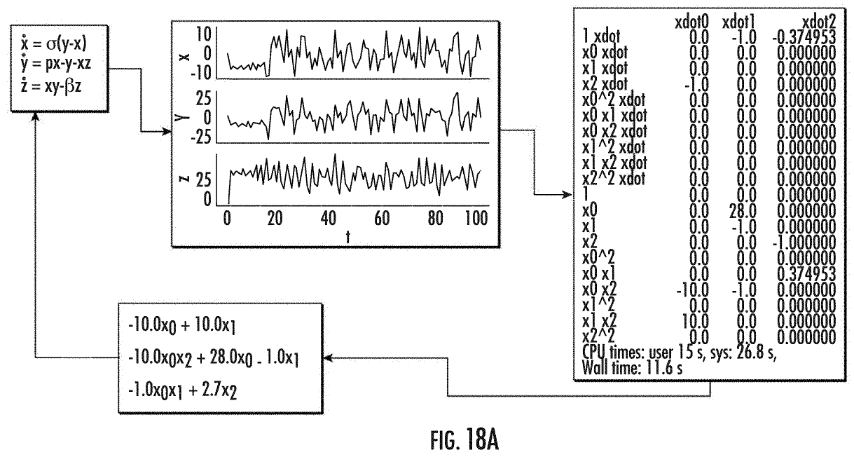

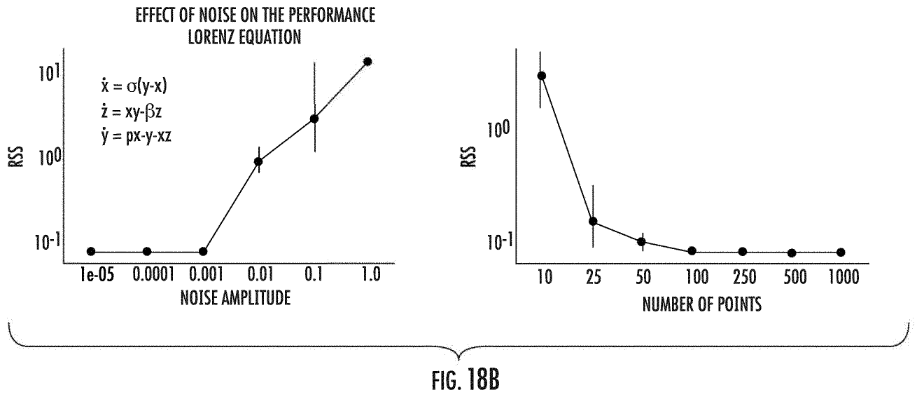

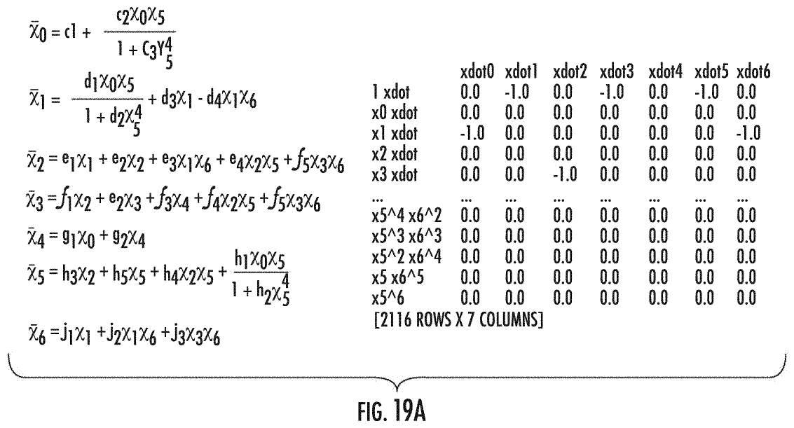

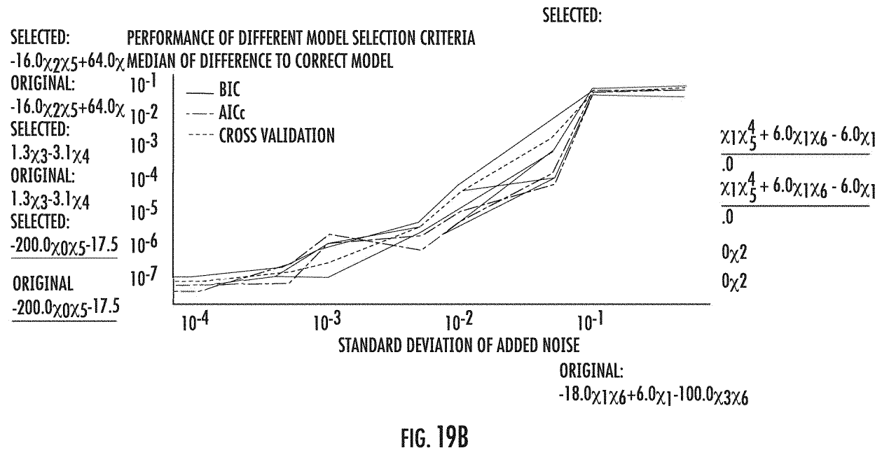

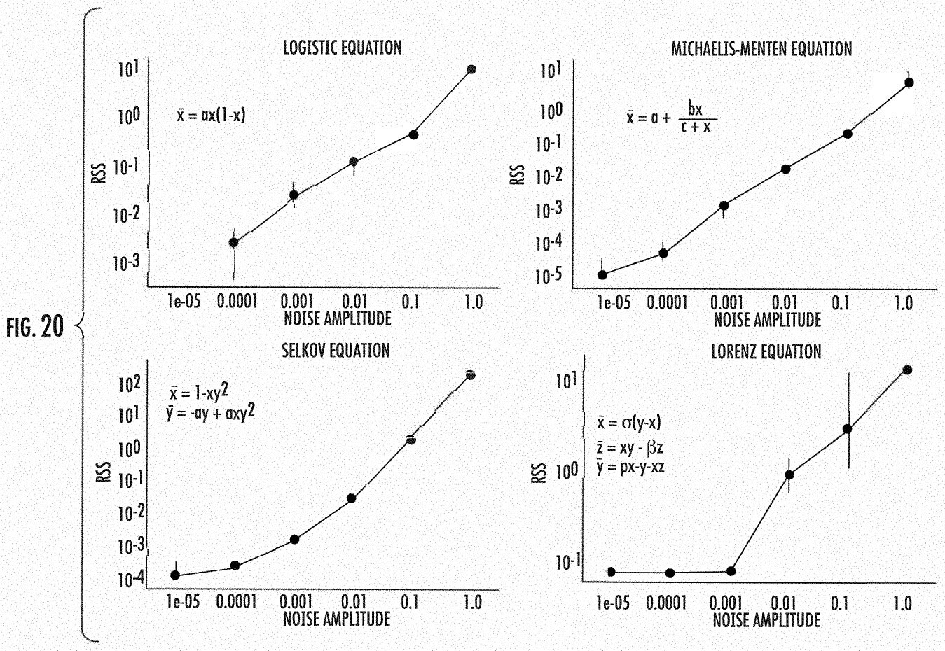

FIG. 17 depicts a first model discovery example using processes according to some embodiments (Michaelis-Menten Equation). FIGS. 18A and 18B depict a second model discovery example using processes according to some embodiments (Lorenz Equation). FIGS. 19A and 19B depict a third model discovery example using processes according to some embodiments (Glycolysis model). FIG. 20 depicts graphical representations of different model performances.

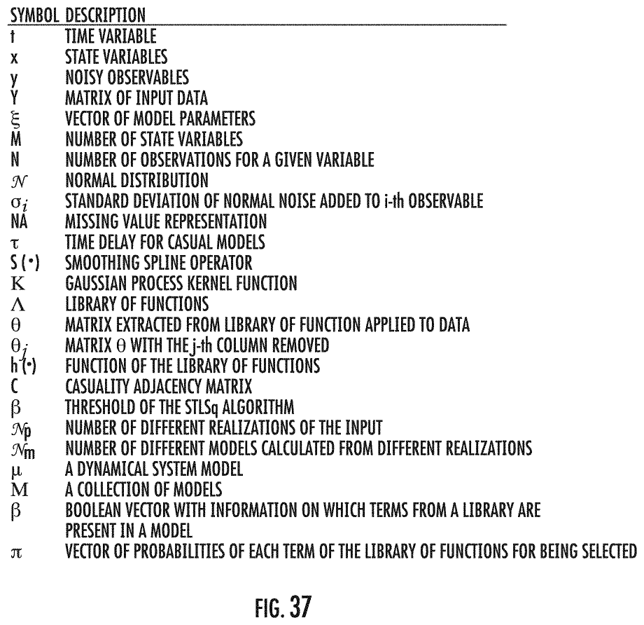

FIG. 37 provides a listing of symbol definitions used in various processes according to the present disclosure.

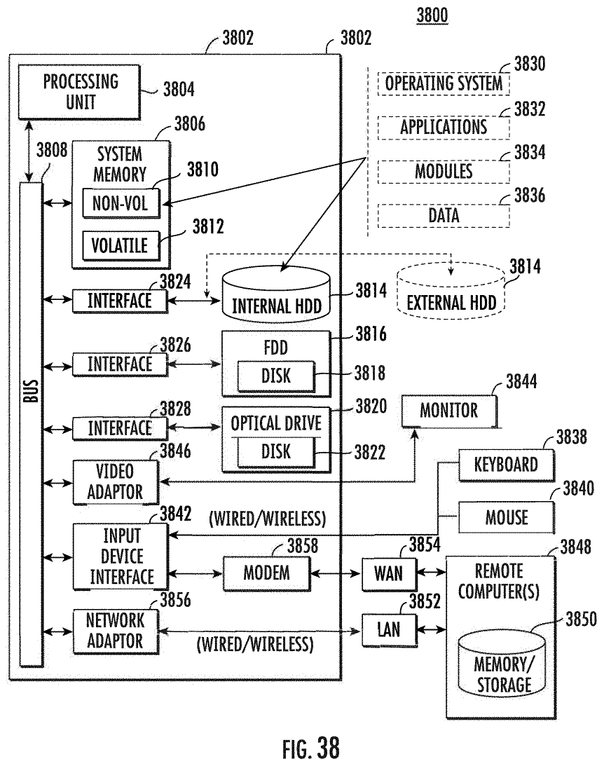

FIG. 38 illustrates an embodiment of an exemplary computing architecture 3800 suitable for implementing various embodiments as previously described. In various embodiments, the computing architecture 3800 may comprise or be implemented as part of an electronic device. In some embodiments, the computing architecture 3800 may be representative, for example, of computing device 110. The embodiments are not limited in this context.

As used in this application, the terms “system” and “component” and “module” are intended to refer to a computer-related entity, either hardware, a combination of hardware and software, software, or software in execution, examples of which are provided by the exemplary computing architecture 3800. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 3800 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computing architecture 3800.

As shown in FIG. 38, the computing architecture 3800 comprises a processing unit 3804, a system memory 3806 and a system bus 3808. The processing unit 3804 may be a commercially available processor and may include dual microprocessors, multi-core processors, and other multi-processor architectures.

The system bus 3808 provides an interface for system components including, but not limited to, the system memory 3806 to the processing unit 3804. The system bus 3808 can be any of several types of bus structure that may further interconnect to a memory bus (with or without a memory controller), a peripheral bus, and a local bus using any of a variety of commercially available bus architectures. Interface adapters may connect to the system bus 3808 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The system memory 3806 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid-state memory devices (e.g., USB memory, solid-state drives (SSD) and any other type of storage media suitable for storing information. In the illustrated embodiment shown in FIG. 38, the system memory 3806 can include non-volatile memory 3810 and/or volatile memory 3812. A basic input/output system (BIOS) can be stored in the non-volatile memory 3810.

The computer 3802 may include various types of computer-readable storage media in the form of one or more lower speed memory units, including an internal (or external) hard disk drive (HDD) 3814, a magnetic floppy disk drive (FDD) 3816 to read from or write to a removable magnetic disk 3811, and an optical disk drive 3820 to read from or write to a removable optical disk 3822 (e.g., a CD-ROM or DVD). The HDD 3814, FDD 3816 and optical disk drive 3820 can be connected to the system bus 3808 by an HDD interface 3824, an FDD interface 3826 and an optical drive interface 3828, respectively. The HDD interface 3824 for external drive implementations can include at least one or both of Universal Serial Bus (USB) and IEEE 1114 interface technologies.

The drives and associated computer-readable media provide volatile and/or nonvolatile storage of data, data structures, computer-executable instructions, and so forth. For example, a number of program modules can be stored in the drives and memory units 3810, 3812, including an operating system 3830, one or more application programs 3832, other program modules 3834, and program data 3836. In one embodiment, the one or more application programs 3832, other program modules 3834, and program data 3836 can include, for example, the various applications and/or components of computing device 110.

A user can enter commands and information into the computer 3802 through one or more wired/wireless input devices, for example, a keyboard 3838 and a pointing device, such as a mouse 3840. These and other input devices are often connected to the processing unit 3804 through an input device interface 3842 that is coupled to the system bus 3808, but can be connected by other interfaces.

A monitor 3844 or other types of display device is also connected to the system bus 3808 via an interface, such as a video adaptor 3846. The monitor 3844 may be internal or external to the computer 3802. In addition to the monitor 3844, a computer typically includes other peripheral output devices, such as speakers, printers, and so forth.

The computer 3802 may operate in a networked environment using logical connections via wired and/or wireless communications to one or more remote computers, such as a remote computer 3848. The remote computer 3848 can be a workstation, a server computer, a router, a personal computer, portable computer, microprocessor-based entertainment appliance, a peer device or other common network node, and typically includes many or all of the elements described relative to the computer 3802, although, for purposes of brevity, only a memory/storage device 3850 is illustrated. The logical connections depicted include wired/wireless connectivity to a local area network (LAN) 3852 and/or larger networks, for example, a wide area network (WAN) 3854. Such LAN and WAN networking environments are commonplace in offices and companies, and facilitate enterprise-wide computer networks, such as intranets, all of which may connect to a global communications network, for example, the Internet.

The computer 3802 is operable to communicate with wired and wireless devices or entities using the IEEE 802 family of standards, such as wireless devices operatively disposed in wireless communication (e.g., IEEE 802.115 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.11x (a, b, g, n, etc.) to provide secure, reliable, fast wireless connectivity. A Wi-Fi network can be used to connect computers to each other, to the Internet, and to wire networks (which use IEEE 802.3-related media and functions).

Numerous specific details have been set forth herein to provide a thorough understanding of the embodiments. It will be understood by those skilled in the art, however, that the embodiments may be practiced without these specific details. In other instances, well-known operations, components, and circuits have not been described in detail so as not to obscure the embodiments. It can be appreciated that the specific structural and functional details disclosed herein may be representative and do not necessarily limit the scope of the embodiments.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Unless specifically stated otherwise, it may be appreciated that terms such as “processing,” “computing,” “calculating,” “determining,” or the like, refer to the action and/or processes of a computer or computing system, or similar electronic computing device, that manipulates and/or transforms data represented as physical quantities (e.g., electronic) within the computing system's registers and/or memories into other data similarly represented as physical quantities within the computing system's memories, registers or other such information storage, transmission or display devices. The embodiments are not limited in this context.

It should be noted that the methods described herein do not have to be executed in the order described, or in any particular order. Moreover, various activities described with respect to the methods identified herein can be executed in a serial or parallel fashion.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein, will be apparent to those of skill in the art upon reviewing the above description. Thus, the scope of various embodiments includes any other applications in which the above compositions, structures, and methods are used.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. An apparatus, comprising:
at least one processor;
a memory coupled to the at least one processor, the memory comprising instructions that, when executed by the at least one processor, cause the at least one processor to perform a chronic kidney and/or end-stage renal diseases (CKD/ESRD) condition analysis process to determine a CKD/ESRD condition model configured to model a CKD/ESRD condition, the vascular calcification analysis process to:
receive input data associated with at least one patient,
perform a dynamical system learner process to build a collection of dynamical system models,
determine a model rank for at least a portion of the collection of dynamical system models, and
determine an optimal dynamical system model for modeling the CKD/ESRD condition for the at least one patient.

2. The apparatus of claim 1, the instructions, when executed by the at least one processor, to cause the at least one processor to pre-process the input data to impute missing values.

3. The apparatus of claim 1, the instructions, when executed by the at least one processor, to cause the at least one processor to perform a causal analysis of the input data to generate causal information.

4. The apparatus of claim 3, the causal information comprising a causal diagram.

5. The apparatus of claim 1, the model rank configured to indicate model performance for dynamical relationships between variables in the input data.

6. The apparatus of claim 1, the collection of dynamical system models to model one or more of the following variables: pre-treatment pulse pressure (P), Neutrophils-Lymphocytes ratio (ρ_{NL}), Serum calcium concentration (C_{Ca}), Intact Parathyroid Hormone (C_{PTH}), Serum albumin concentration (g/dL) (C_{Ab}), Serum phosphorus concentration (C_{P}), or Alkaline Phosphatase (C_{AP}).

7. The apparatus of claim 1, the instructions, when executed by the at least one processor, to cause the at least one processor to:
receive patient information for a patient;
analyze the patient information using one of the collections of dynamical models to predict a CKD/ESRD condition process for the patient based on modeled variables.

8. The apparatus of claim 1, the input data comprising a time series of system observables and a library of functions configured as an operator on the input data.

9. The apparatus of claim 1, the dynamical system models comprising differential equations that describe a time evolution of at least one variable of the input data.

10. A computer-implemented method to perform a chronic kidney and/or end-stage renal diseases (CKD/ESRD) condition analysis process to determine a CKD/ESRD condition model configured to model a CKD/ESRD condition, the method comprising:
receiving input data associated with at least one patient,
performing a dynamical system learner process to build a collection of dynamical system models,
determining a model rank for at least a portion of the collection of dynamical system models, and
determining an optimal dynamical system model for modeling the CKD/ESRD condition for the at least one patient.

11. The method of claim 10, the instructions, when executed by the at least one processor, to cause the at least one processor to pre-process the input data to impute missing values.

12. The method of claim 10, the instructions, when executed by the at least one processor, to cause the at least one processor to perform a causal analysis of the input data to generate causal information.

13. The method of claim 12, the causal information comprising a causal diagram.

14. The method of claim 10, the model rank is configured to indicate model performance for dynamical relationships between variables in the input data.

15. The method of claim 10, the collection of dynamical system models to model one or more of the following variables: pre-treatment pulse pressure (P), Neutrophils-Lymphocytes ratio (ρ_{NL}), Serum calcium concentration (C_{Ca}), Intact Parathyroid Hormone (C_{PTH}), Serum albumin concentration (g/dL) (C_{Ab}), Serum phosphorus concentration (C_{P}), or Alkaline Phosphatase (C_{AP}).

16. A computer-implemented method of vascular calcification analysis, the method comprising, via a processor of a computing device:
determining a vascular calcification model configured to model vascular calcification of a virtual patient to determine a causal relationship between at least one patient characteristic and a vascular calcification indicator; and
generate a causal relationship structure configured to visualize a causal relationship between the at least one patient characteristic and the vascular calcification indicator.

17. The method of claim 1, the vascular calcification indicator comprising one of pulse pressure (PP) or pulse wave velocity.

18. The method of claim 1, the at least one patient characteristic comprising at least one of parathyroid hormones (PTH), calcium (Ca), phosphate (PO4), calcium-phosphate product (CaPO4), neutrophil-lymphocyte ratio (NLR), and albumin (Alb).

19. The method of claim 1, the causal relationship structure comprising at least one of a causality fingerprint or a causality pathway map.

20. The method of claim 1, further comprising administering a treatment regimen based on the causal relationship structure.
